(19) United States
(12) Patent Application Publication
(10) Pub. No.: US 2025/0114359 A1
(43) Pub. Date: Apr. 10, 2025

(54) METHODS FOR INHIBITING PARASITIC GLUCOKINASE AND/OR HEXOKINASE

(71) Applicant: UNIVERSITY OF SOUTH CAROLINA, COLUMBIA, SC (US)

(72) Inventor: Edward L. D'Antonio, BLUFFTON, SC (US)

(21) Appl. No.: 18/906,487

(22) Filed: Oct. 4, 2024

Related U.S. Application Data
(60) Provisional application No. 63/587,770, filed on Oct. 4, 2023.

(51) Int. Cl. A61K 31/515; A61K 31/121; A61K 31/122; A61K 31/343; A61K 31/4152; A61K 31/4196; A61K 31/426

(52) U.S. Cl. CPC A61K 31/515; A61K 31/121; A61K 31/122; A61K 31/343; A61K 31/4152; A61K 31/4196; A61K 31/426

(57) ABSTRACT

Inhibitors directed at enzymes of the pentose phosphate pathway of protozoan parasites, e.g., Trypanosoma cruzi glucokinase, are described. Inhibitors are derivatives of gossypol (2,2′-Bis(formyl-1,6,7-trihydroxy-5-isopropyl-3-methylnaphthalene). The inhibitors can specifically target glucokinase and hexokinase of Trypanosoma cruzi (T. cruzi), Trypanosoma brucei (T. brucei), and Leishmania spp. and are useful in treatment of parasitic diseases including Chagas' disease and African sleeping sickness, among others.

### CROSS REFERENCE TO RELATED APPLICATIONS

This application claims filing benefit of U.S. Provisional Patent Application Ser. No. 63/587,770, filed on Oct. 4, 2023, which is incorporated herein by reference.

### BACKGROUND

Kinetoplastid parasites, such as Trypanosoma cruzi (T. cruzi), Trypanosoma brucei (T. brucei), and Leishmania spp., utilize and depend on the pentose phosphate pathway (PPP) for the reducing agent NADPH and also rely on the PPP for support for nucleic acid and nucleotide biosynthesis. The PPP is essential for these organisms and obstruction of the pathway leads to cell death and can be caused by inhibition of an enzyme required in the PPP such as glucose 6-phosphate dehydrogenase. In order to create a suitable therapeutic, an inhibitor should selectively block the targeted parasite homologue and avoid cross-reactivity with the human homologue (i.e., bind weaker or not bind at all), giving rise to a good selectivity ratio.

T. cruzi is the causative agent for Chagas' disease and benznidazole and nifurtimox are the two main clinically available treatments available in Latin America. These drugs have the potential for resistance because they were developed over 35 years ago and alternative drugs have not emerged. T. brucei is the causative agent for human African sleeping sickness and various drugs are available, such as pentamidine, suramin, eflornithine, and melarsoprol. Leishmania spp. are protozoan parasites causing Leishmaniasis and medical intervention requires treatment such as pentavalent antimony-based medicines, or more expensive treatments such as amphotericin B, miltefosine, or paramomycin. The drugs for these kinetoplastid diseases all require substantial improvements in their tolerability, safety, and efficacy.

A need exists for new materials that strongly bind to drug targets found in these parasites. Such a need includes inhibitors of the glucose kinase enzymes, hexokinase and glucokinase. Methods for inhibiting such parasitic enzymes would be of great benefit in treating multiple parasite-caused diseases.

### SUMMARY

Objects and advantages of the invention will be set forth in part in the following description, or may be obvious from the description, or may be learned through practice of the invention.

Methods are provided for inhibiting parasitic hexokinase and glucokinase through delivery to a parasite a compound as described herein.

Also disclosed are methods for treating a human subject infected with a kinetoplastid parasite through administering to the human a pharmaceutical composition that includes a pharmaceutically acceptable carrier and a compound as described herein.

In one embodiment, the compound can be a gossypol derivative having the following general structure:

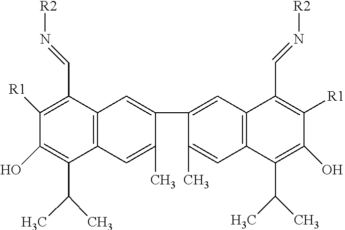

in which R1 is oxygen or hydroxyl and R2 includes a substituted phenyl group, a substituted conjugated or unconjugated cyclopentyl or cyclooctyl group, a substituted cyclohexyl group, or a substituted or unsubstituted C3-C7 group.

In one embodiment, the compound can be a gossypol derivative having the following general structure:

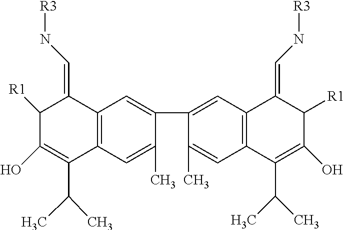

in which R1 is oxygen or hydroxyl and R3 is selected from one of the following:

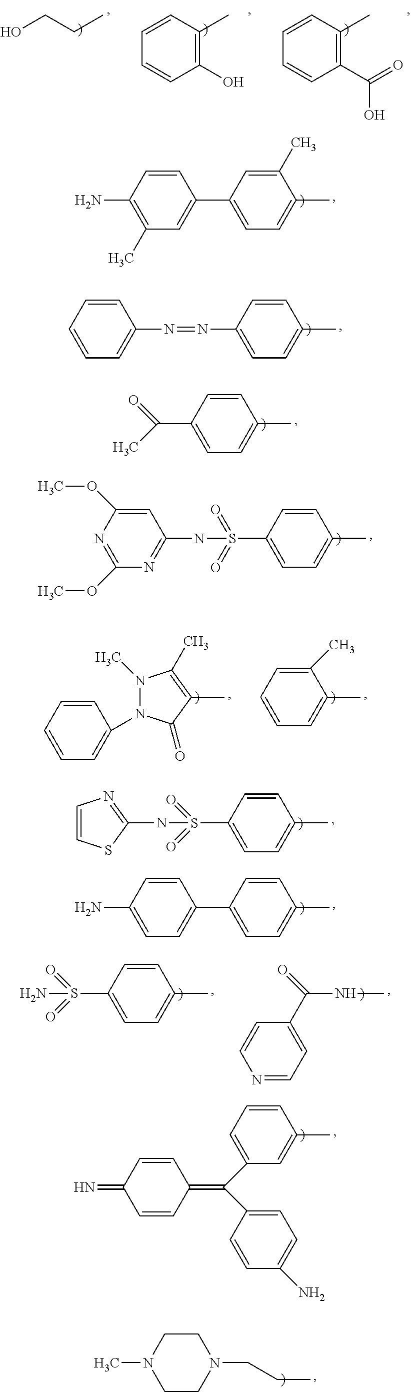

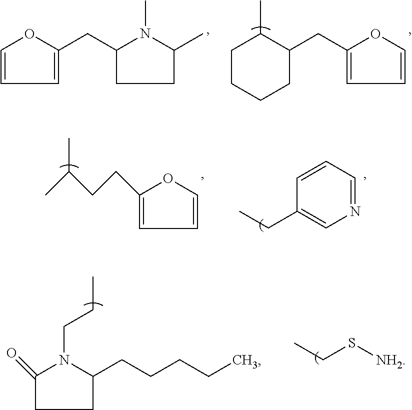

In another embodiment, the compound can have the following structure:

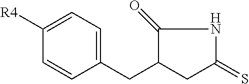

in which R4 is selected from hydrogen, hydroxyl, methoxy, or amine and optionally, the phenyl group is instead a naphthyl group.

In another embodiment, the compound can have one of the following structures:

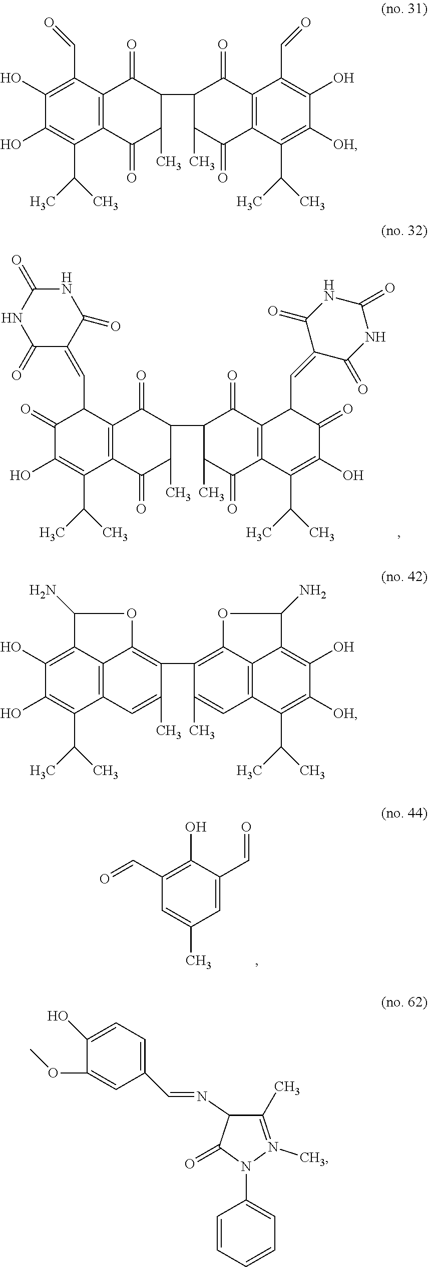

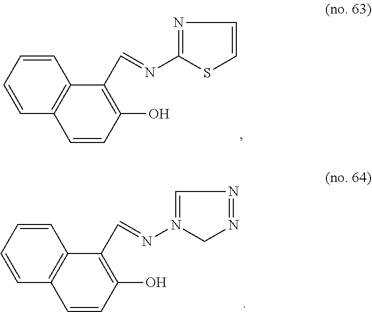

Other features and aspects of the present invention are discussed in greater detail below.

### BRIEF DESCRIPTION OF THE DRAWINGS

FIG. 1 provides structures of Scaffold 1 type inhibitors as described herein.

FIG. 2 provides structures of Scaffold 2 type inhibitors as described herein.

FIG. 3 provides structures of Scaffold 3 type inhibitors as described herein.

FIG. 4 provides structures of further examples of inhibitors as described herein.

FIG. 5 provides type 1 and type 2 Dixon plots for inhibitor no. 1 as described herein.

FIG. 6 provides type 1 and type 2 Dixon plots for a comparative compound that does not exhibit inhibition.

FIG. 7 provides type 1 and type 2 Dixon plots for inhibitor no. 3 as described herein.

FIG. 8 provides type 1 and type 2 Dixon plots for inhibitor no. 4 as described herein.

FIG. 9 provides type 1 and type 2 Dixon plots for inhibitor no. 5 as described herein.

FIG. 10 provides type 1 and type 2 Dixon plots for inhibitor no. 6 as described herein.

FIG. 11 provides type 1 and type 2 Dixon plots for inhibitor no. 7 as described herein.

FIG. 12 provides type 1 and type 2 Dixon plots for inhibitor no. 8 as described herein.

FIG. 13 provides type 1 and type 2 Dixon plots for inhibitor no. 9 as described herein.

FIG. 14 provides type 1 and type 2 Dixon plots for inhibitor no. 10 as described herein.

FIG. 15 provides type 1 and type 2 Dixon plots for inhibitor no. 11 as described herein.

FIG. 16 provides type 1 and type 2 Dixon plots for inhibitor no. 12 as described herein.

FIG. 17 provides type 1 and type 2 Dixon plots for inhibitor no. 13 as described herein.

FIG. 18 provides type 1 and type 2 Dixon plots for inhibitor no. 14 as described herein.

FIG. 19 provides type 1 and type 2 Dixon plots for inhibitor no. 17 as described herein.

FIG. 20 provides type 1 and type 2 Dixon plots for inhibitor no. 18 as described herein.

FIG. 21 provides type 1 and type 2 Dixon plots for inhibitor no. 19 as described herein.

FIG. 22 provides type 1 and type 2 Dixon plots for inhibitor no. 20 as described herein.

FIG. 23 provides type 1 and type 2 Dixon plots for inhibitor no. 21 as described herein.

FIG. 24 provides type 1 and type 2 Dixon plots for inhibitor no. 22 as described herein.

FIG. 25 provides type 1 and type 2 Dixon plots for inhibitor no. 23 as described herein.

FIG. 26 provides type 1 and type 2 Dixon plots for inhibitor no. 25 as described herein.

FIG. 27 provides type 1 and type 2 Dixon plots for inhibitor no. 15 as described herein.

FIG. 28 provides type 1 and type 2 Dixon plots for inhibitor no. 16 as described herein.

FIG. 29 provides type 1 and type 2 Dixon plots for inhibitor no. 26 as described herein.

FIG. 30 provides type 1 and type 2 Dixon plots for inhibitor no. 27 as described herein.

FIG. 31 provides type 1 and type 2 Dixon plots for inhibitor no. 28 as described herein.

FIG. 32 provides type 1 and type 2 Dixon plots for inhibitor no. 29 as described herein.

FIG. 33 provides type 1 and type 2 Dixon plots for inhibitor no. 30 as described herein.

FIG. 34 provides type 1 and type 2 Dixon plots for inhibitor no. 31 as described herein.

FIG. 35 provides type 1 and type 2 Dixon plots for inhibitor no. 33 as described herein.

FIG. 36 provides type 1 and type 2 Dixon plots for inhibitor no. 35 as described herein.

FIG. 37 provides type 1 and type 2 Dixon plots for inhibitor no. 36 as described herein.

FIG. 38 provides type 1 and type 2 Dixon plots for inhibitor no. 37 as described herein.

FIG. 39 provides type 1 and type 2 Dixon plots for inhibitor no. 38 as described herein.

FIG. 40 provides type 1 and type 2 Dixon plots for inhibitor no. 39 as described herein.

FIG. 41 provides type 1 and type 2 Dixon plots for inhibitor no. 40 as described herein.

FIG. 42 provides type 1 and type 2 Dixon plots for inhibitor no. 41 as described herein.

FIG. 43 provides type 1 and type 2 Dixon plots for inhibitor no. 42 as described herein.

FIG. 45 provides type 1 and type 2 Dixon plots for inhibitor no. 45 as described herein.

FIG. 46 provides type 1 and type 2 Dixon plots for inhibitor no. 46 as described herein.

FIG. 47 provides type 1 and type 2 Dixon plots for inhibitor no. 47 as described herein.

FIG. 48 provides type 1 and type 2 Dixon plots for inhibitor no. 49 as described herein.

FIG. 49 provides type 1 and type 2 Dixon plots for inhibitor no. 50 as described herein.

FIG. 50 provides type 1 and type 2 Dixon plots for inhibitor no. 51 as described herein.

FIG. 51 provides type 1 and type 2 Dixon plots for inhibitor no. 53 as described herein.

FIG. 52 provides type 1 and type 2 Dixon plots for inhibitor no. 54 as described herein.

FIG. 53 provides type 1 and type 2 Dixon plots for inhibitor no. 55 as described herein.

FIG. 54 provides type 1 and type 2 Dixon plots for inhibitor no. 57 as described herein.

FIG. 55 provides type 1 and type 2 Dixon plots for inhibitor no. 58 as described herein.

FIG. 56 provides type 1 and type 2 Dixon plots for inhibitor no. 59 as described herein.

FIG. 57 provides type 1 and type 2 Dixon plots for inhibitor no. 60 as described herein.

FIG. 58 provides type 1 and type 2 Dixon plots for inhibitor no. 61 as described herein.

FIG. 59 provides type 1 and type 2 Dixon plots for inhibitor no. 63 as described herein.

FIG. 60 provides type 1 and type 2 Dixon plots for inhibitor no. 64 as described herein.

FIG. 61 provides type 1 and type 2 Dixon plots for inhibitor no. 65 as described herein.

FIG. 62 provides type 1 and type 2 Dixon plots for inhibitor no. 66 as described herein.

FIG. 63 provides type 1 and type 2 Dixon plots for inhibitor no. 67 as described herein.

FIG. 64 provides type 1 and type 2 Dixon plots for inhibitor no. 68 as described herein.

FIG. 65 provides type 1 and type 2 Dixon plots for inhibitor no. 69 as described herein.

FIG. 66 provides type 1 and type 2 Dixon plots for inhibitor no. 70 as described herein.

FIG. 67 provides type 1 and type 2 Dixon plots for inhibitor no. 71 as described herein.

FIG. 68 provides type 1 and type 2 Dixon plots for inhibitor no. 72 as described herein.

FIG. 69 provides type 1 and type 2 Dixon plots for inhibitor no. 73 as described herein.

FIG. 70 provides type 1 and type 2 Dixon plots for inhibitor no. 74 as described herein.

FIG. 71 provides type 1 and type 2 Dixon plots for inhibitor no. 75 as described herein.

FIG. 72 provides type 1 and type 2 Dixon plots for inhibitor no. 76 as described herein.

FIG. 73 provides type 1 and type 2 Dixon plots for inhibitor no. 77 as described herein.

FIG. 74 provides type 1 and type 2 Dixon plots for inhibitor no. 78 as described herein.

FIG. 75 provides type 1 and type 2 Dixon plots for inhibitor no. 79 as described herein.

FIG. 76 provides type 1 and type 2 Dixon plots for inhibitor no. 81 as described herein.

FIG. 77 provides type 1 and type 2 Dixon plots for inhibitor no. 83 as described herein.

FIG. 78 provides type 1 and type 2 Dixon plots for inhibitor no. 85 as described herein.

FIG. 79 provides type 1 and type 2 Dixon plots for inhibitor no. 86 as described herein.

FIG. 80 provides type 1 and type 2 Dixon plots for inhibitor no. 87 as described herein.

### DETAILED DESCRIPTION

Reference now will be made to the embodiments of the invention, one or more examples of which are set forth below. Each example is provided by way of an explanation of the invention, not as a limitation of the invention. In fact, it will be apparent to those skilled in the art that various modifications and variations can be made in the invention without departing from the scope or spirit of the invention. For instance, features illustrated or described as one embodiment can be used on another embodiment to yield still a further embodiment. Thus, it is intended that the present invention cover such modifications and variations as come within the scope of the appended claims and their equivalents. It is to be understood by one of ordinary skill in the art that the present discussion is a description of exemplary embodiments only, and is not intended as limiting the broader aspects of the present invention, which broader aspects are embodied exemplary constructions.

Chemical elements are discussed in the present disclosure using their common chemical abbreviation, such as commonly found on a periodic table of elements. For example, hydrogen is represented by its common chemical abbreviation H; helium is represented by its common chemical abbreviation He; and so forth.

The term “organic” is used herein to refer to a class of chemical compounds that are comprised of carbon atoms. For example, an “organic polymer” is a polymer that includes carbon atoms in the polymer backbone, but may also include other atoms either in the polymer backbone and/or in sidechains extending from the polymer backbone (e.g., oxygen, nitrogen, sulfur, etc.).

As used herein, the term “related compounds thereof” refers to compounds that have the basic structure of the base compound with substituted atom(s) and/or substituted side groups.

The term “pharmaceutically effective amount” refers to the amount of a drug or pharmaceutical agent that will elicit the biological or medical response of a tissue, system, animal, or human that is being sought by a researcher or clinician. This amount can be a therapeutically effective amount.

The term “pharmaceutically acceptable carrier” is used herein to refer to a carrier that is useful in preparing a pharmaceutical composition that is generally safe, non-toxic and neither biologically nor otherwise undesirable, and is acceptable for veterinary use as well as human pharmaceutical use. A “pharmaceutically acceptable carrier” as used in the specification and claims can include both one and more than one such carrier. The term “pharmaceutically acceptable” refers to a carrier that is compatible with the other ingredients of the formulation and not deleterious to the recipient thereof.

The terms “administration of” or “administering a” pharmaceutical composition should be understood to mean providing a pharmaceutical composition to an individual in need of treatment in a form that can be introduced into that individual's body in a therapeutically useful form and therapeutically useful amount, including, but not limited to: oral dosage forms, such as tablets, capsules, syrups, suspensions, and the like; injectable dosage forms, such as IV, IM, or IP, and the like; transdermal dosage forms, including creams, jellies, powders, or patches; buccal dosage forms; inhalation powders, sprays, suspensions, and the like; and rectal suppositories.

The term “treatment” or “treating” means any administration of a pharmaceutical composition to obtain a desired pharmacologic and/or physiologic effect. The effect may be prophylactic in terms of completely or partially preventing a disease or symptom thereof, and/or may be therapeutic in terms of a partial or complete cure for a disease and/or adverse effect attributable to the disease.

Treatment includes (a) inhibiting the disease in the subject that is experiencing or displaying the pathology or symptomatology of the diseased (i.e., arresting further development of the pathology and/or symptomatology), or (b) ameliorating the disease in the subject that is experiencing or displaying the pathology or symptomatology of the diseased (i.e., reversing the pathology and/or symptomatology).

Compounds and methods are generally provided that can be used against parasitic enzymes, e.g., Trypanosoma cruzi glucokinase (TcGlcK), that are found in protozoan parasites. The compounds may serve as viable substitutes for currently used drugs in clinical settings. The compounds described herein include potent inhibitors that are effective for use in inhibition of T. cruzi as well as T. brucei glucokinase and/or hexokinase and/or glucokinase or hexokinase of the Leishmania species (e.g., Leishmania braziliensis). The compounds described herein may offer an alternative to the mainstream drugs that are used in the clinic for diseases of the trypanosome, such as American Trypanosomiasis (Chagas' disease), Human African Trypanosomiasis (Human African Sleeping Sickness), and Leishmaniasis caused by parasites T. cruzi, T. brucei, and Leishmania spp., respectively.

Disclosed inhibitors can generally have a weight average molecular weight, M_{w} of about 1500 Da or less, such as about 1300 Da or less, or about 1100 Da or less, such as from about 200 Da to about 1500 Da. In some embodiments, disclosed inhibitors can be considered small molecule inhibitors, i.e., having a M_{w} of about 500 Da or less, such as from about 200 Da to about 500 Da.

The inhibitors can exhibit an inhibition constant (Kᵢ) of about 1 μM or greater, about 10 μM or greater, or about 50 μM or greater, such as from about 1 μM to about 5000 μM in some embodiments. The inhibition constant can be determined by development of a Dixon plot (either type 1 or type 2) according to standard practice, and as utilized herein refers to the inhibitor concentration at which half maximum inhibition is determined for a 100 μL/10 mM format in DMSO.

Inhibitors described herein have been derived from gossypol (2,2′-Bis(formyl-1,6,7-trihydroxy-5-isopropyl-3-methylnaphthalene), which is a pigment found in the cotton plant (among others). It is a natural defensive compound that inhibits several dehydrogenase enzymes in insects and can interfere with sperm development and function, leading to infertility. Gossypol has the following structure:

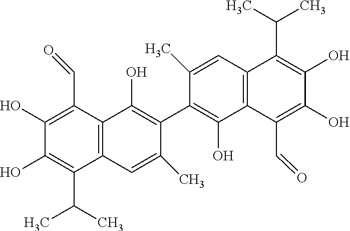 [SMILES: Cc1cc2c(C(C)C)c(O)c(O)c(C=O)c2c(O)c1-c1c(C)cc2c(C(C)C)c(O)c(O)c(C=O)c2c1O]

Disclosed inhibitors include inhibitors having a common scaffold as well as singleton inhibitors. In one embodiment, disclosed inhibitors can have a common scaffold referred to herein as Scaffold 1 inhibitors. Scaffold 1 inhibitors have the following general structure:

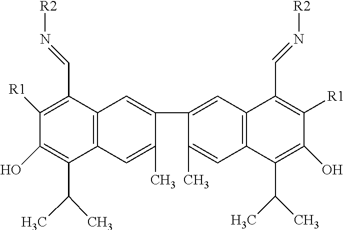

in which R1 is oxygen or hydroxyl and R2 includes a substituted phenyl group, a substituted conjugated or unconjugated cyclopentyl or cyclooctyl group, a substituted cyclohexyl group, or a substituted or unsubstituted C3-C7 group.

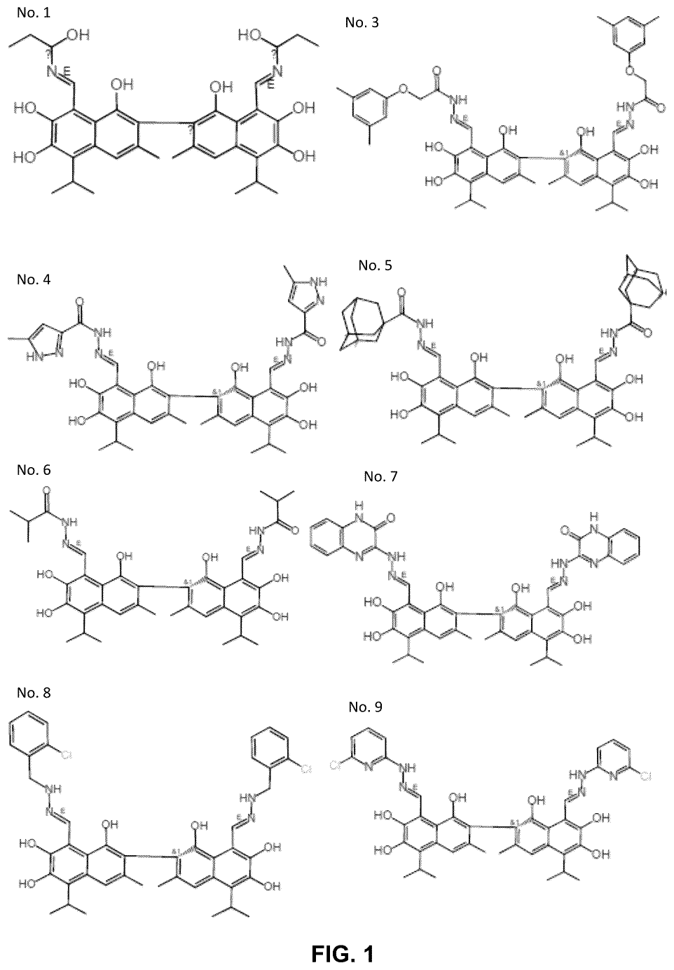

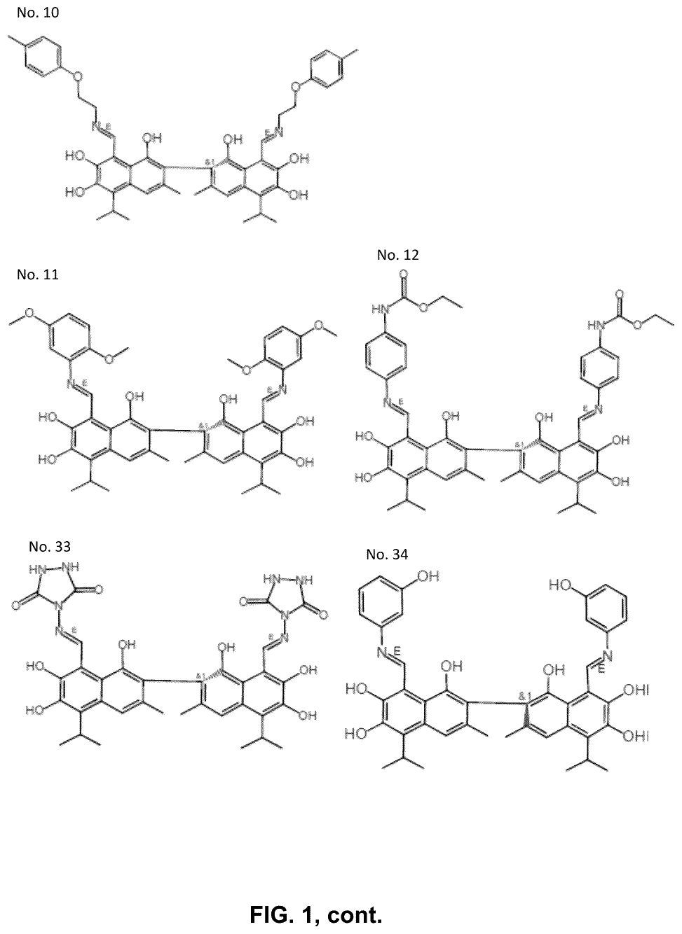

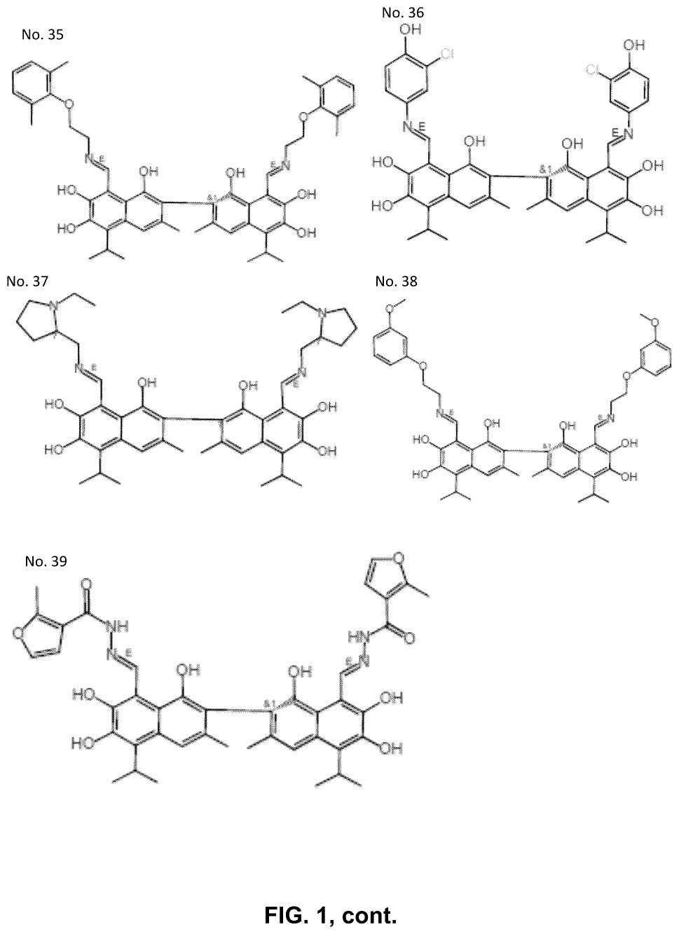

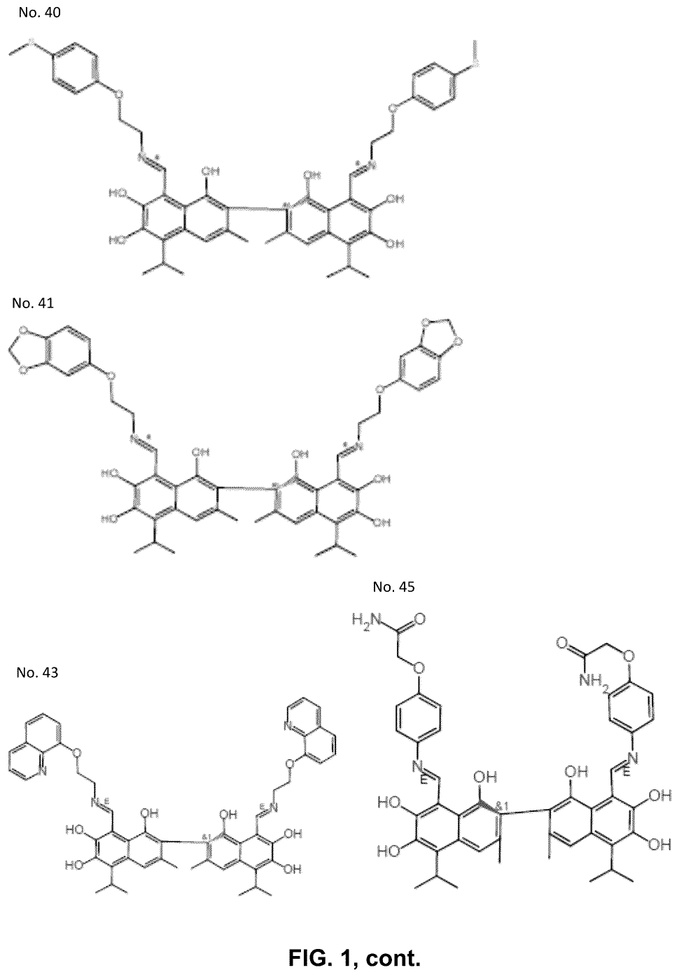

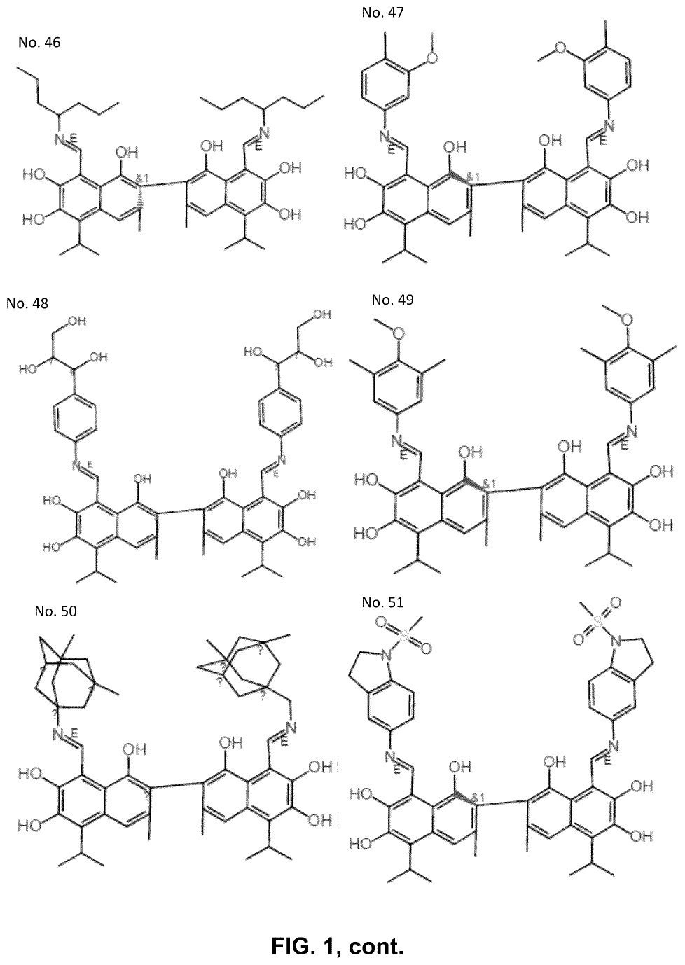

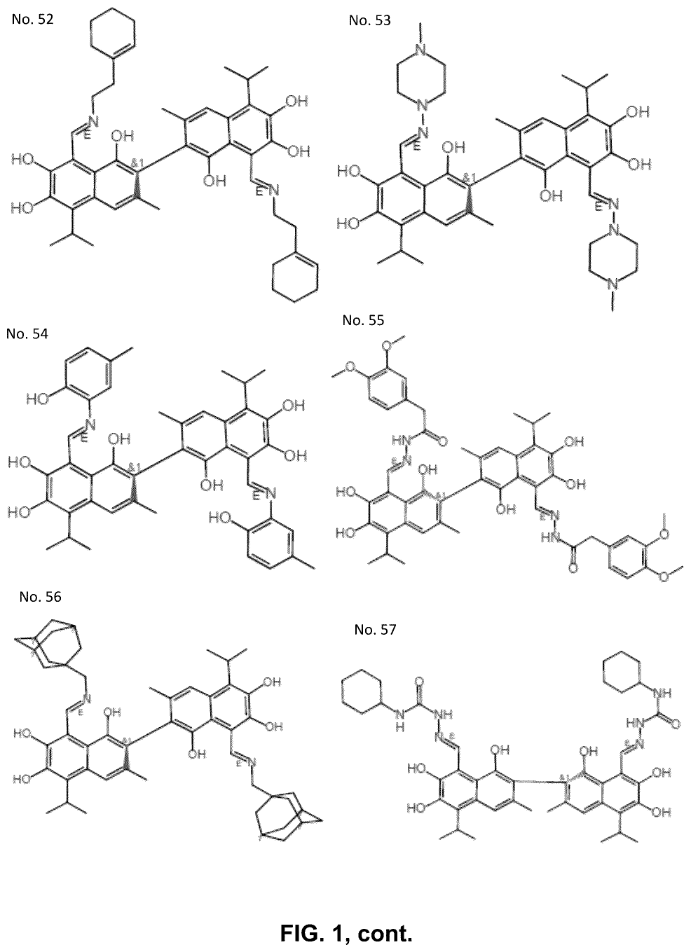

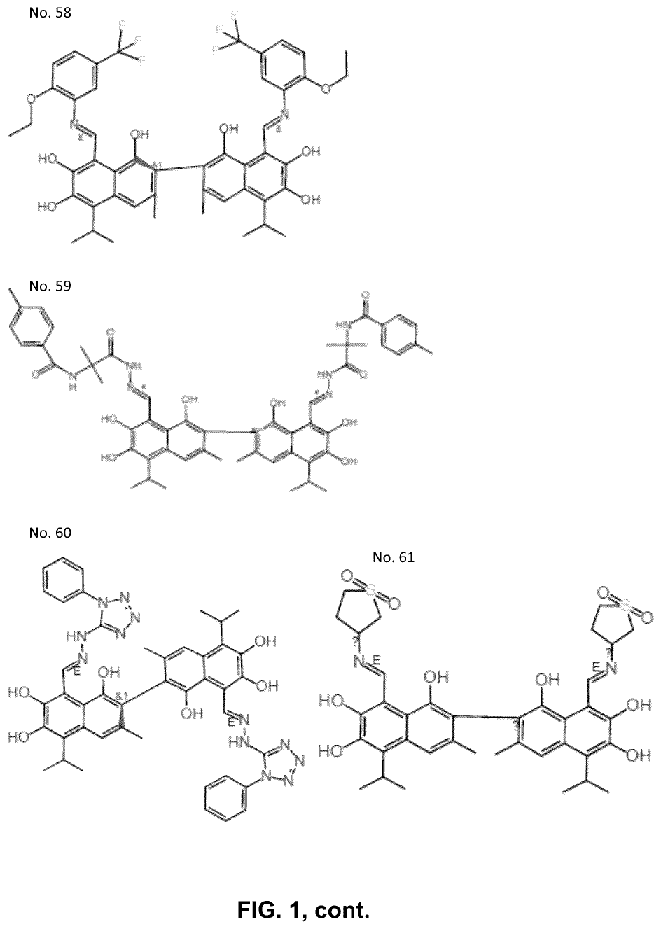

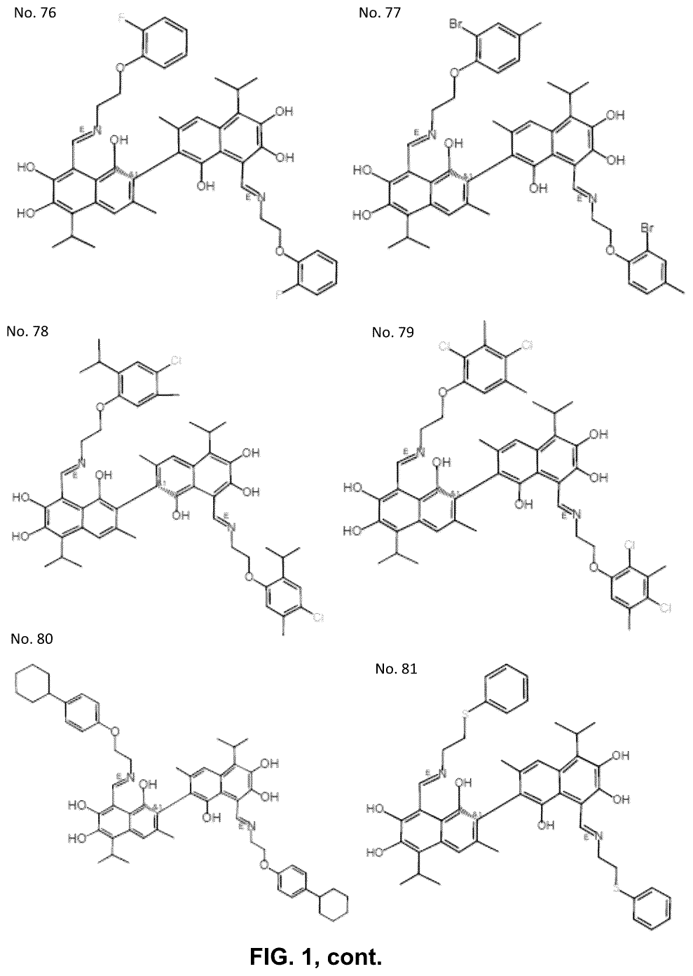

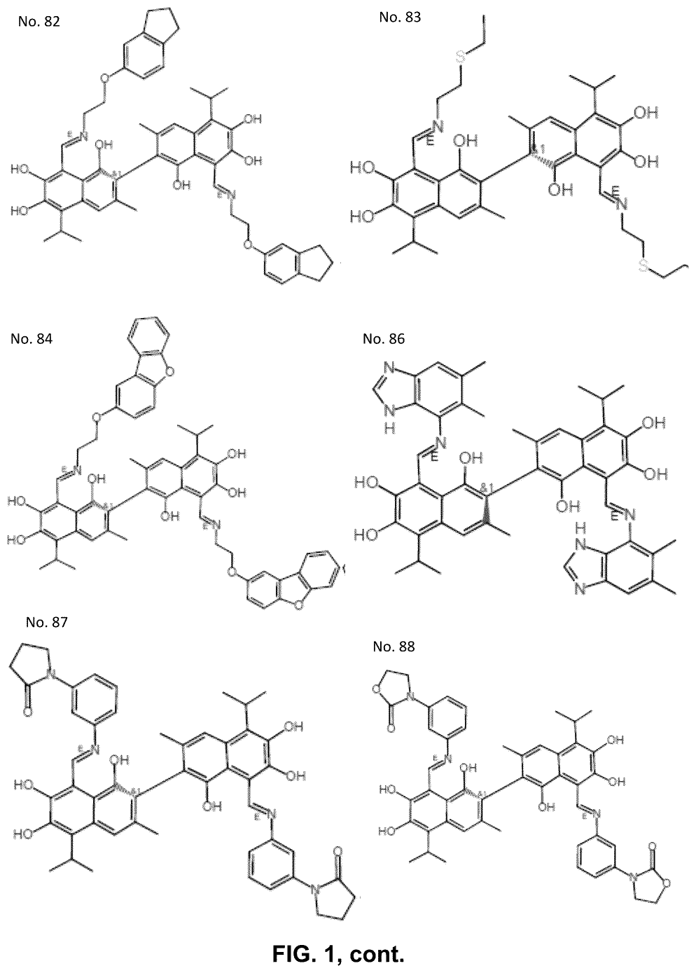

Specific examples of Scaffold 1 inhibitors include inhibitors illustrated in FIG. 1, which are referred to in FIG. 1 by an identifying inhibitor number.

In one embodiment, disclosed inhibitors can have a common scaffold referred to herein as Scaffold 2 inhibitors. Scaffold 2 inhibitors have the following general structure:

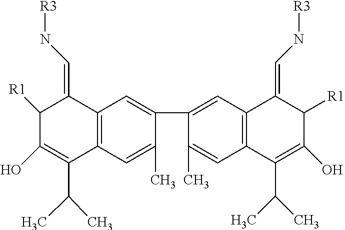

in which R1 is oxygen or hydroxyl and R3 is selected from one of the following:

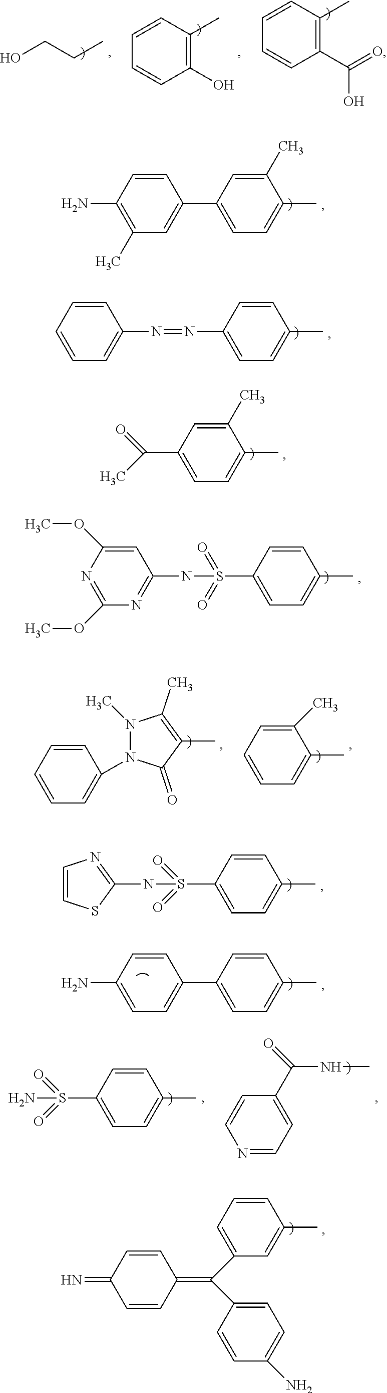

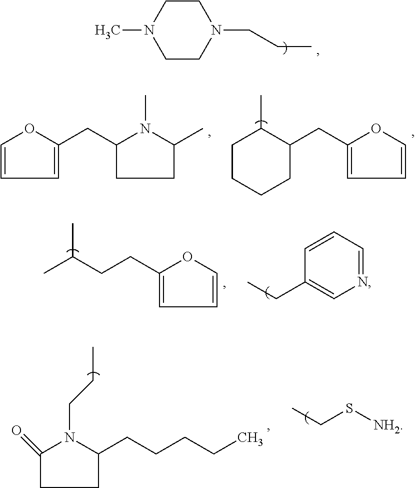

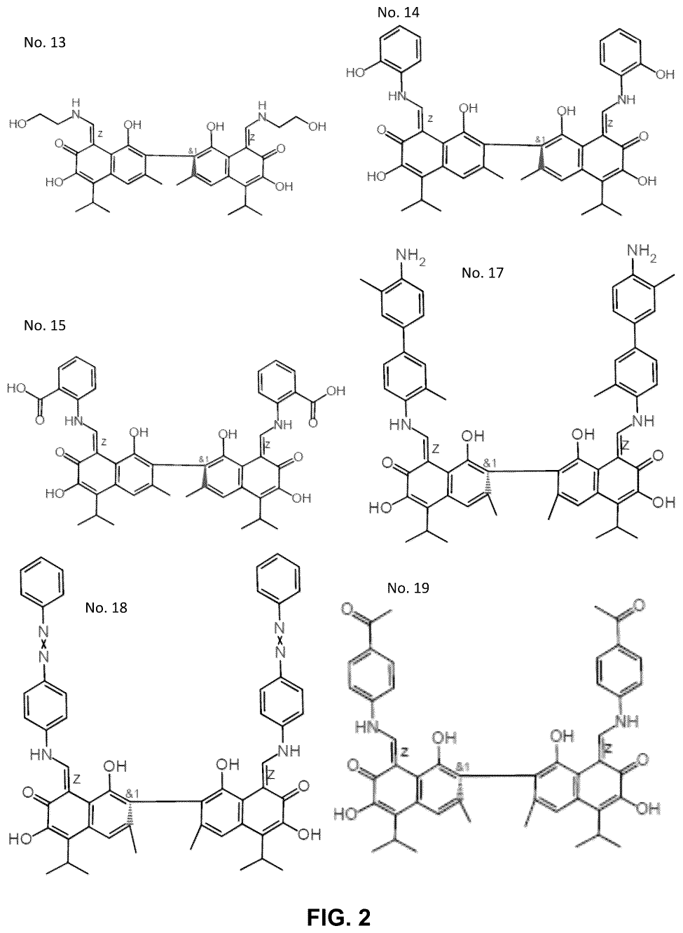

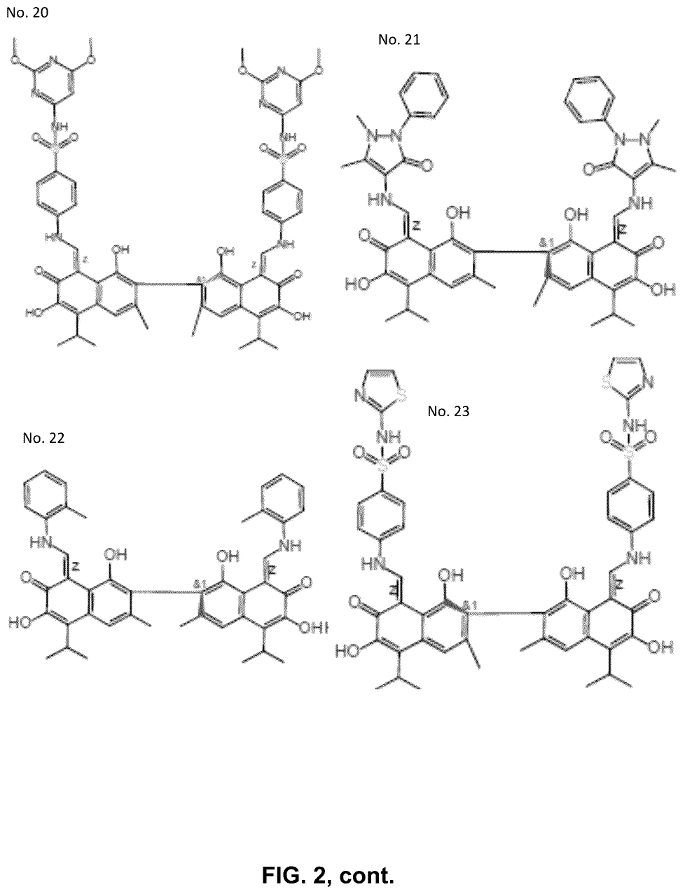

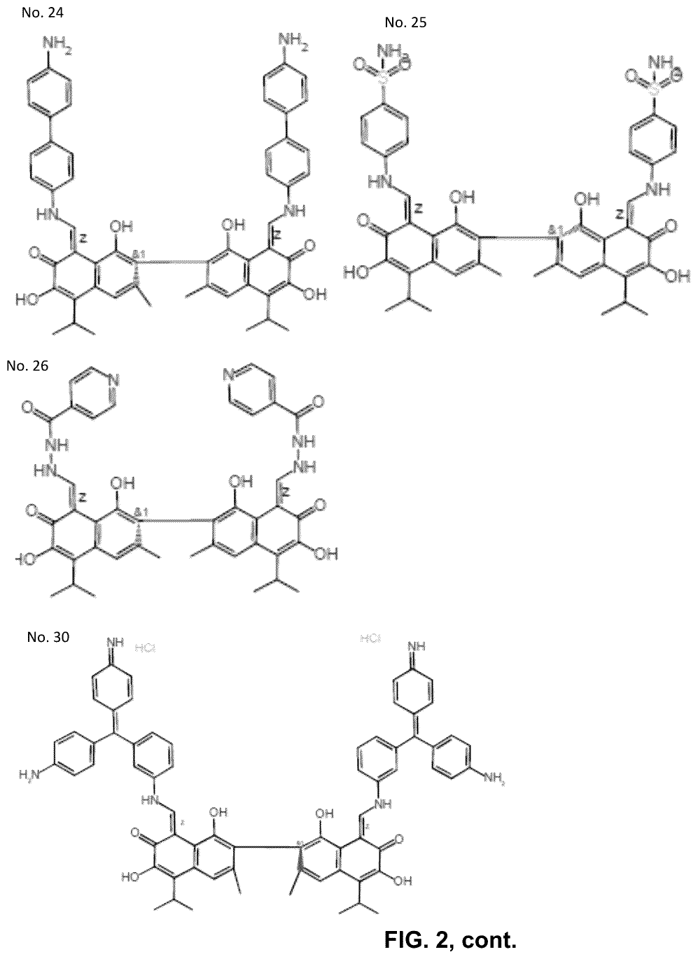

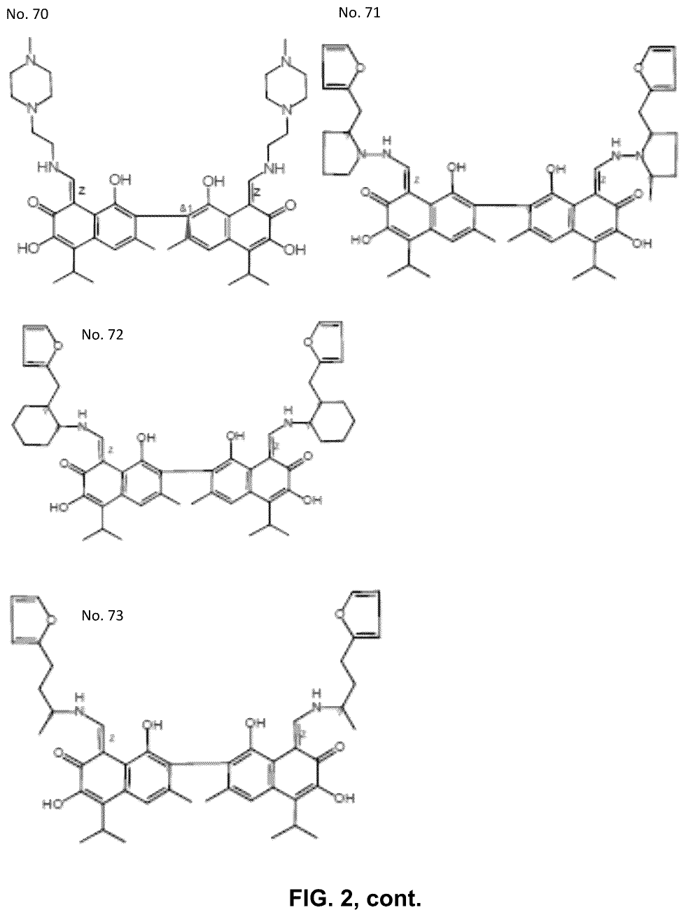

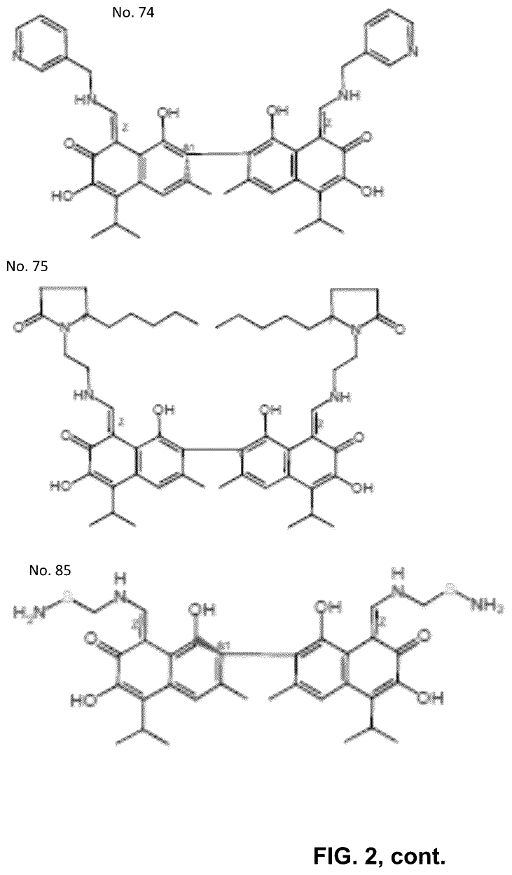

Specific examples of Scaffold 2 inhibitors include inhibitors illustrated in FIG. 2, which are referred to in FIG. 2 by an identifying inhibitor number.

In one embodiment, disclosed inhibitors can have a common scaffold referred to herein as Scaffold 3 inhibitors. Scaffold 3 inhibitors have the following general structure:

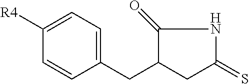

in which R4 is selected from hydrogen, hydroxyl, methoxy, or amine and optionally, the phenyl group is instead a naphthyl group.

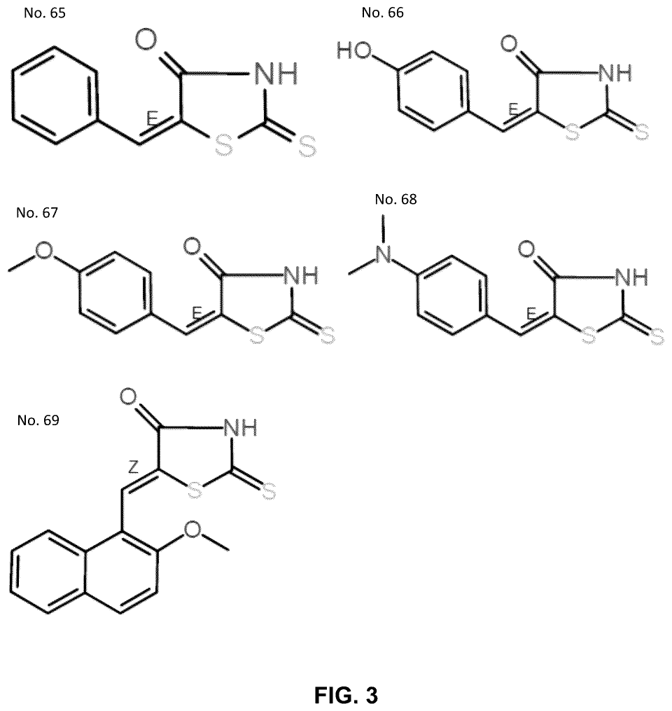

Specific examples of Scaffold 3 inhibitors include inhibitors illustrated in FIG. 3, which are referred to in FIG. 3 by an identifying inhibitor number.

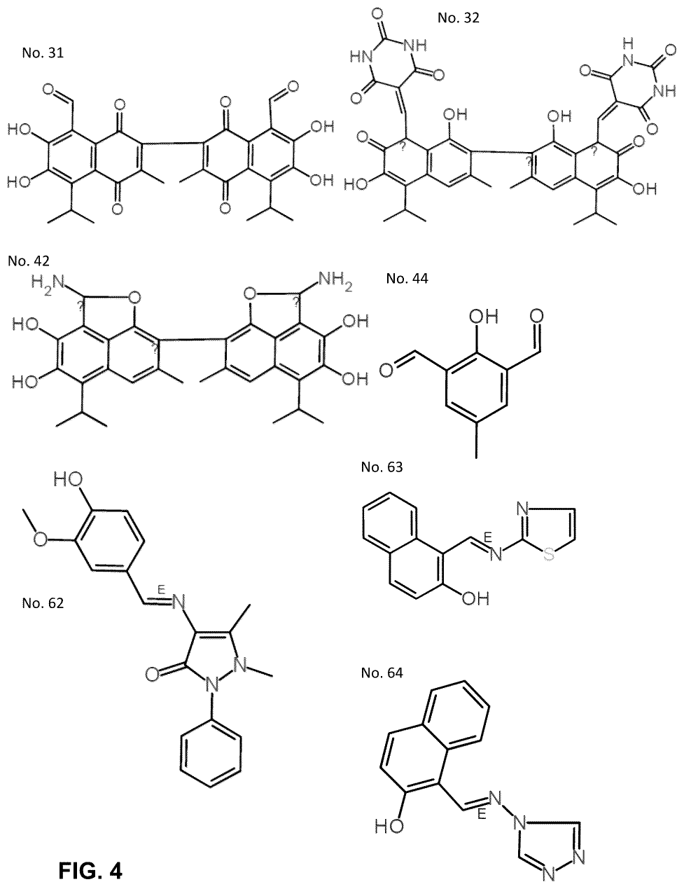

Several singleton inhibitors are also disclosed herein, having the structures as are illustrated in FIG. 4, which are referred to in FIG. 4 by an identifying inhibitor number.

Pharmaceutical compositions are provided that include a pharmaceutically acceptable carrier and at least one compound as disclosed herein, and in one embodiment, a compound having any one of the structures as illustrated in FIG. 1-FIG. 4.

Pharmaceutical compositions may be prepared by any of the methods well known in the art of pharmacy. Pharmaceutical compositions encompass any compositions made by admixing the active ingredients and a pharmaceutically acceptable carrier. The carrier may take a wide variety of forms depending on the form of preparation desired for administration, e.g., oral or parenteral (including intravenous). Thus, the pharmaceutical composition can be presented as discrete units suitable for oral administration such as capsules, cachets, or tablets each containing a predetermined amount of the active ingredients. Further, the composition can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the composition may also be administered by controlled release means and/or delivery devices. The foregoing list is illustrative only and is not intended to be limiting in any way.

Pharmaceutical compositions intended for oral use may contain one or more agents selected from the group consisting of sweetening agents, flavoring agents, coloring agents and preserving agents in order to provide pharmaceutically elegant and palatable preparations. Tablets may contain a composition having at least one of the compounds described herein in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid, or talc. The tablets may be uncoated or they may be coated by known techniques to delay disintegration and absorption in the gastrointestinal tract and thereby provide a sustained action over a longer period. A tablet may be prepared by compression or molding, optionally with one or more accessory ingredients or adjuvants. Compressed tablets may be prepared by compressing, in a suitable machine, at least one of disclosed compounds in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent.

Pharmaceutical compositions for oral use may also be presented as hard gelatin capsules wherein one or more of the disclosed compounds is mixed with an inert solid diluent, for example, calcium carbonate, calcium phosphate or kaolin, or as soft gelatin capsules wherein the compound(s) is/are mixed with water or an oil medium, for example peanut oil, liquid paraffin, or olive oil.

Pharmaceutical compositions can also include aqueous suspensions, which contain the active materials in admixture with excipients suitable for the manufacture of aqueous suspensions. In addition, oily suspensions may be formulated by suspending at least one of the disclosed compounds in a vegetable oil, for example arachis oil, olive oil, sesame oil or coconut oil, or in a mineral oil such as liquid paraffin. Oily suspensions may also contain various excipients. The pharmaceutical composition may also be in the form of oil-in-water emulsions, which may also contain excipients such as sweetening and flavoring agents.

Pharmaceutical compositions can be in the form of a sterile injectable aqueous or oleaginous suspension, or in the form of sterile powders for the extemporaneous preparation of such sterile injectable solutions or dispersions. In all cases, the final injectable form must be sterile and must be effectively fluid for easy injection. The pharmaceutical compositions must be stable under the conditions of manufacture and storage, and should be preserved against the contaminating action of microorganisms such as bacteria and fungi.

Pharmaceutical compositions can be in a form suitable for topical use such as, for example, an aerosol, cream, ointment, lotion, dusting powder, or the like. Further, the compositions can be in a form suitable for use in transdermal devices. These formulations may be prepared via conventional processing methods. As an example, a cream or ointment can be prepared by mixing hydrophilic material and water, together with about 5 wt. % to about 10 wt. % of one or more of the disclosed compound, to produce a cream or ointment having a desired consistency.

Pharmaceutical compositions can also be in a form suitable for rectal administration wherein the carrier is a solid. Suitable carriers include cocoa butter and other materials commonly used in the art.

Both in vitro and in vivo methods for inhibiting a parasitic enzyme are encompassed herein. In one embodiment, the method comprises contacting the parasite or the parasitic enzyme with at least one of the disclosed compounds, and in one embodiment, one of the 83 compounds illustrated in FIG. 1, FIG. 2, FIG. 3, or FIG. 4.

In one embodiment, provided is a method for treating a subject that is infected by a parasitic organism. For instance, a method can include administering to a human diagnosed with a parasitic infection or a disease associated with the parasite a composition comprising a pharmaceutically acceptable carrier and at least one compound as described herein, e.g., a compound as illustrated in FIG. 1, FIG. 2, FIG. 3, or FIG. 4. A parasite that can be affected by an inhibitor as described herein can produce a glucokinase and/or hexokinase and encompassed parasitic organisms can include parasites associated with a disease such as, without limitation, American Trypanosomiasis (Chagas' disease), Human African Trypanosomiasis (African Sleeping Sickness), Leishmaniasis, Malaria, Schistosomaisis (Snail Fever), Filarial diseases, etc. Examples of disease-affected subjects are not limited to humans and can include humans as well as other mammals, e.g., domestic animals such as dogs, cats, etc.

### Examples

Determination of the Kᵢ values for inhibitors was performed by creation of both type 1 and type 2 Dixon plots. The format included 100 μL/10 mM in DMSO. For comparison, a non-inhibitory compound was also examined. The non-inhibitor compound was as follows:

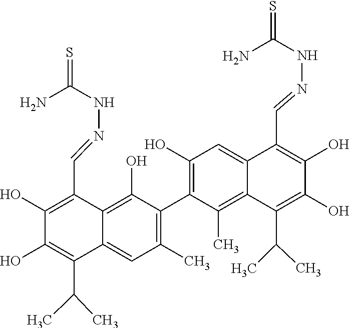 [SMILES: Cc1cc2c(C(C)C)c(O)c(O)c(/C=N/NC(N)=S)c2c(O)c1-c1c(O)cc2c(/C=N/NC(N)=S)c(O)c(O)c(C(C)C)c2c1C]

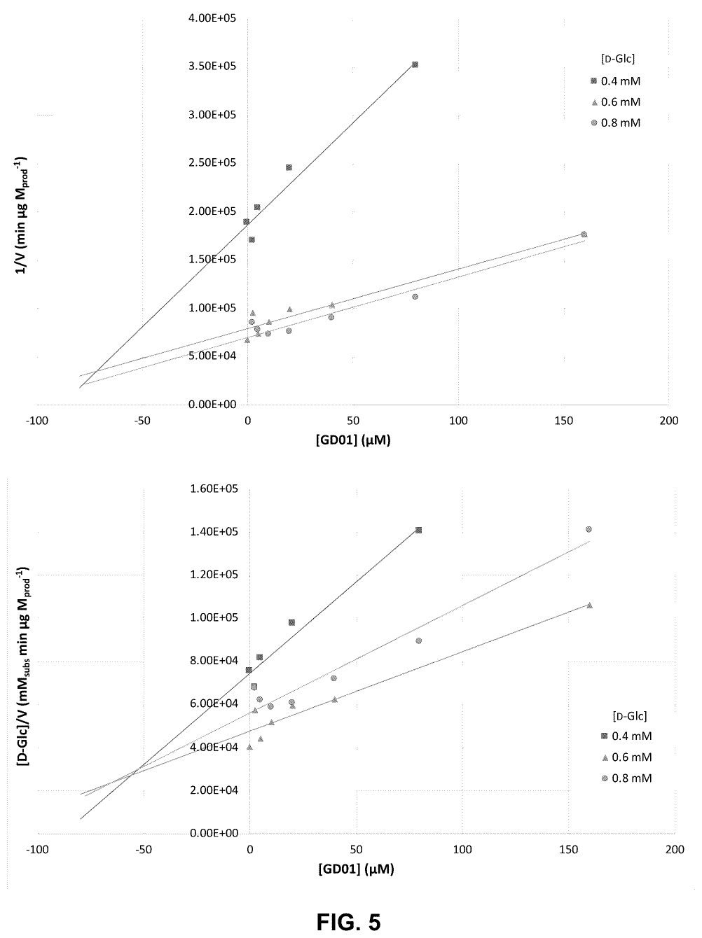

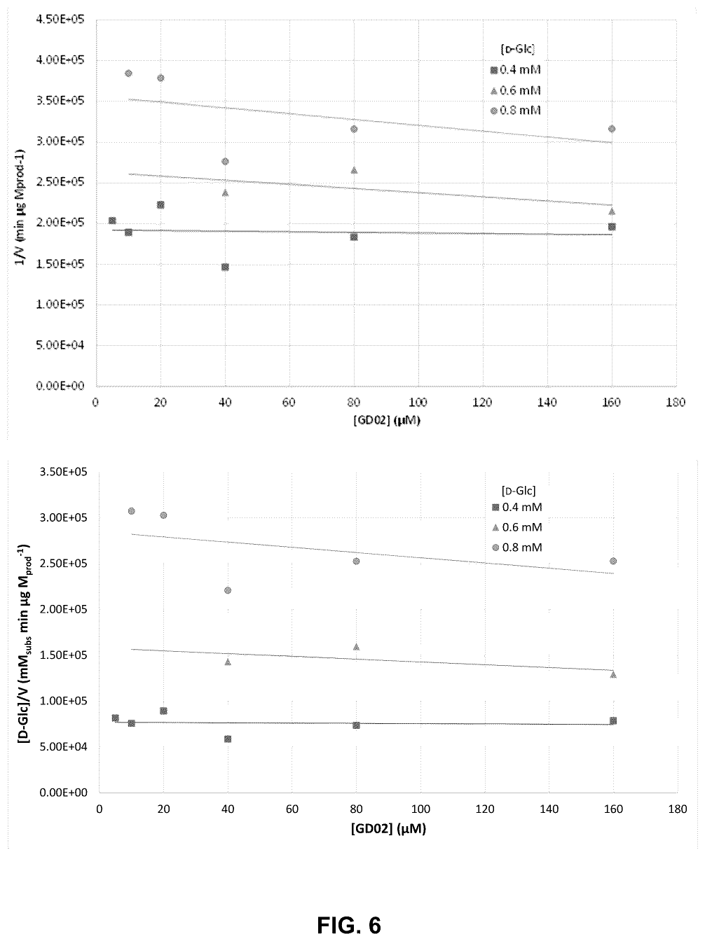

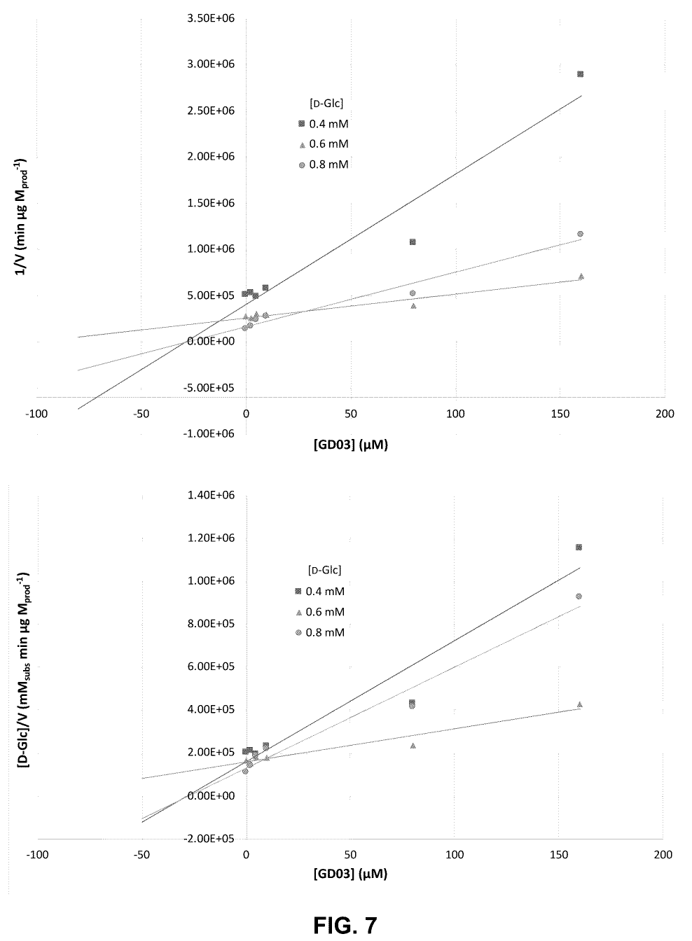

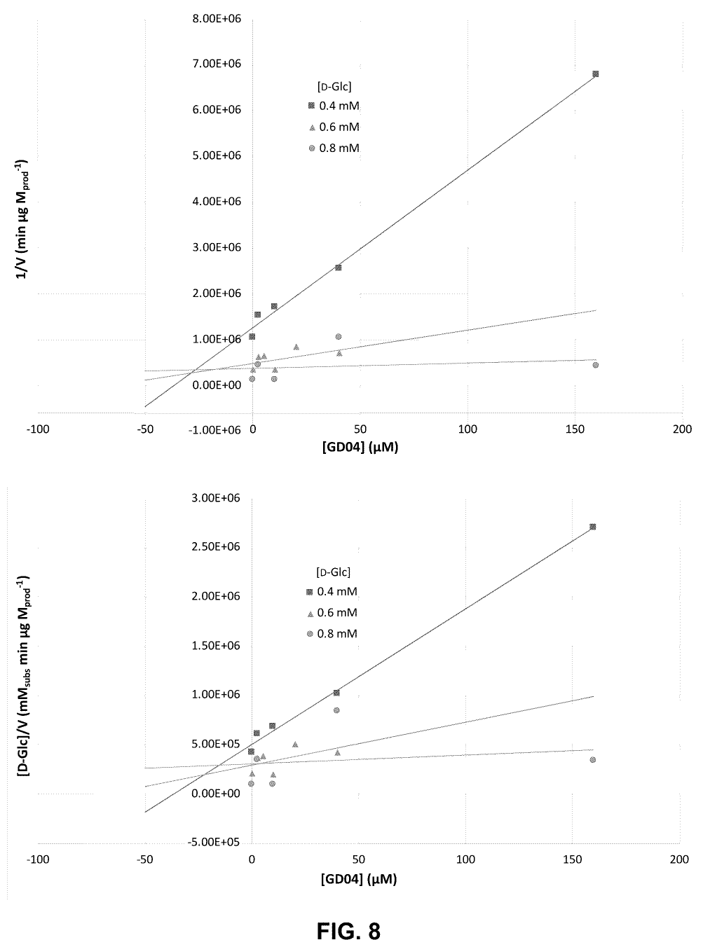

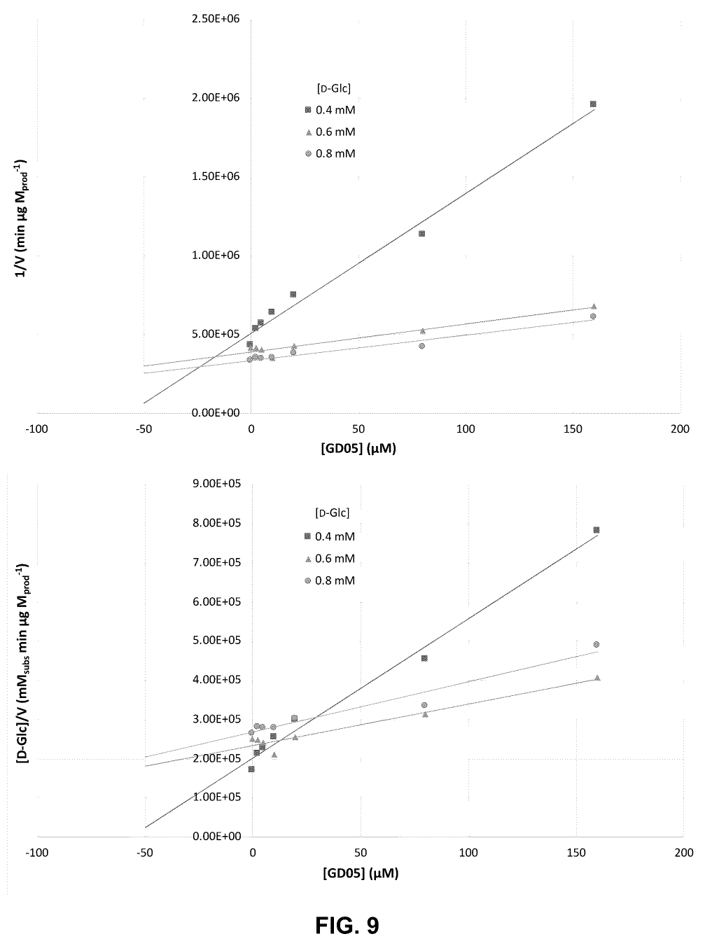

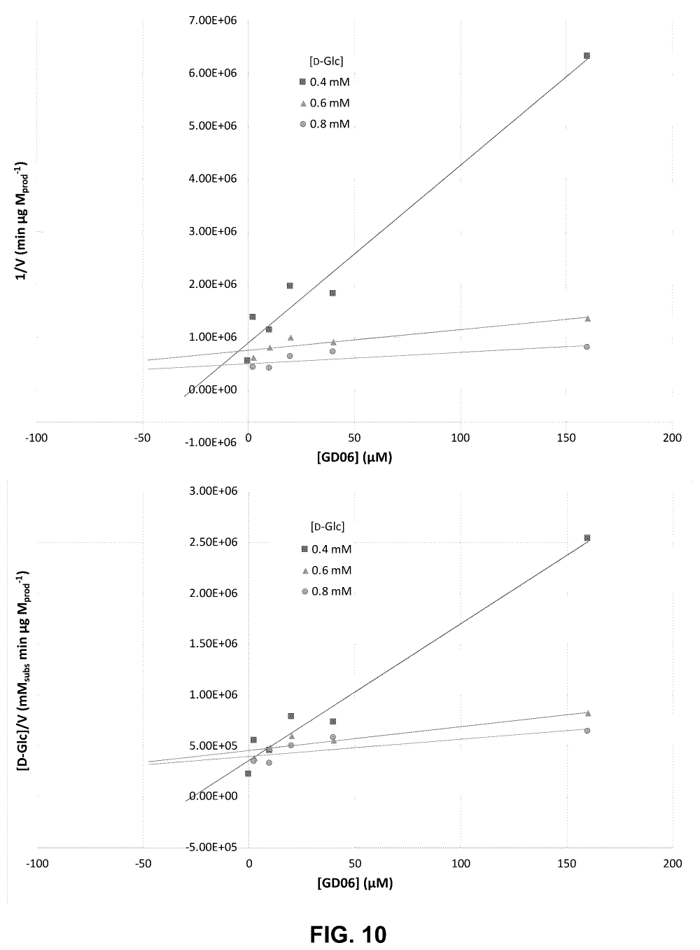

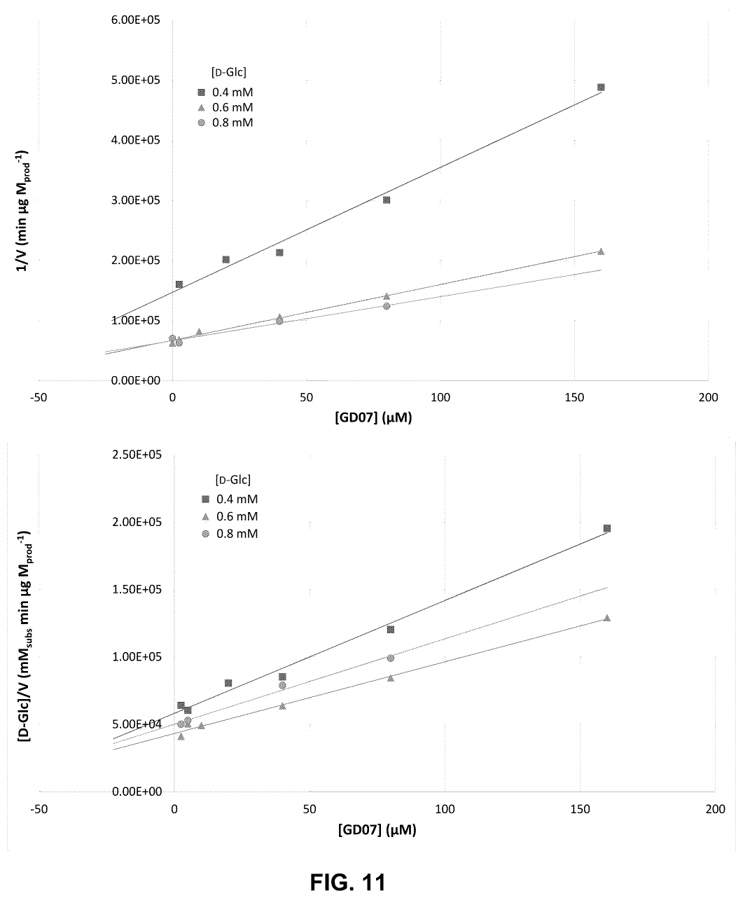

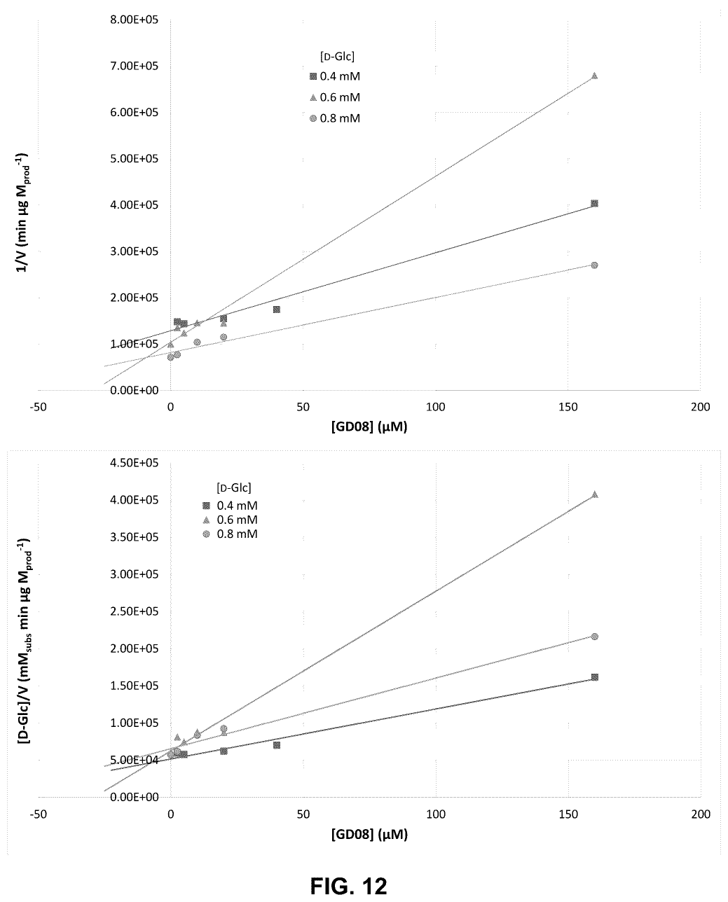

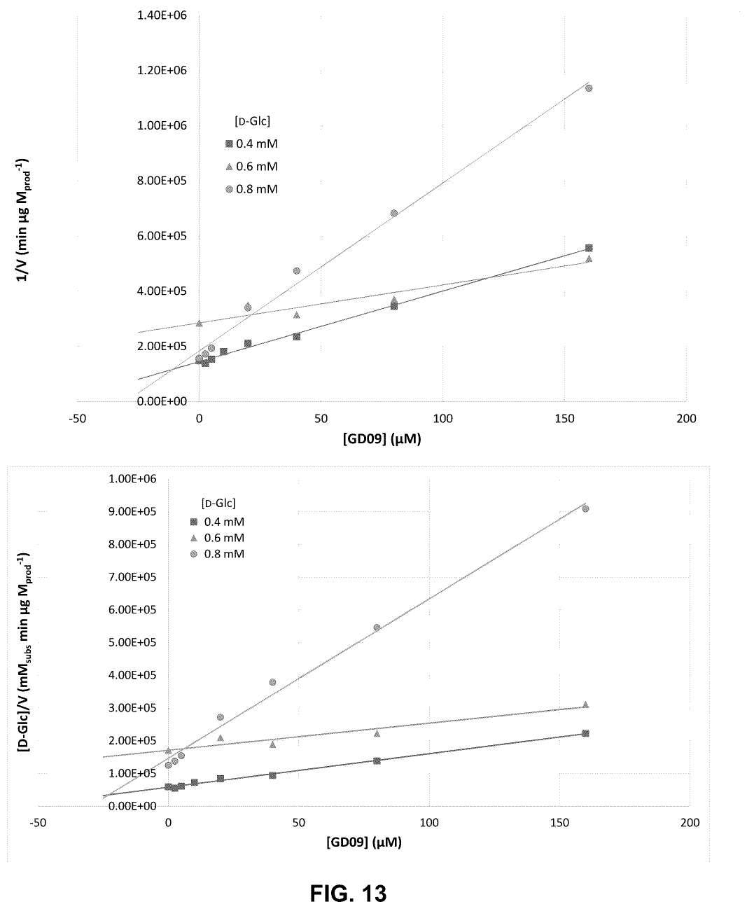

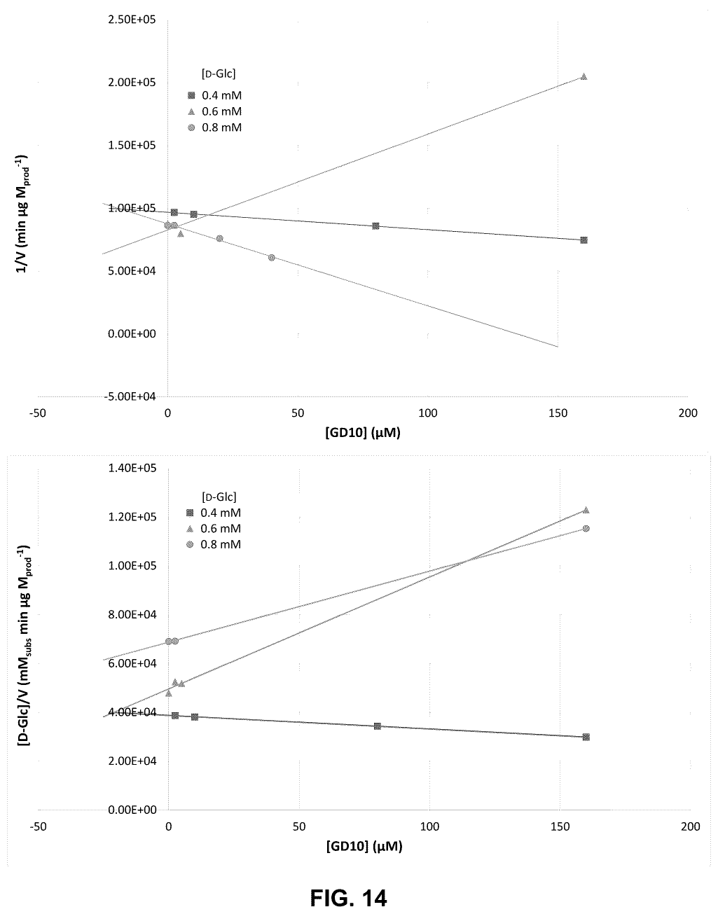

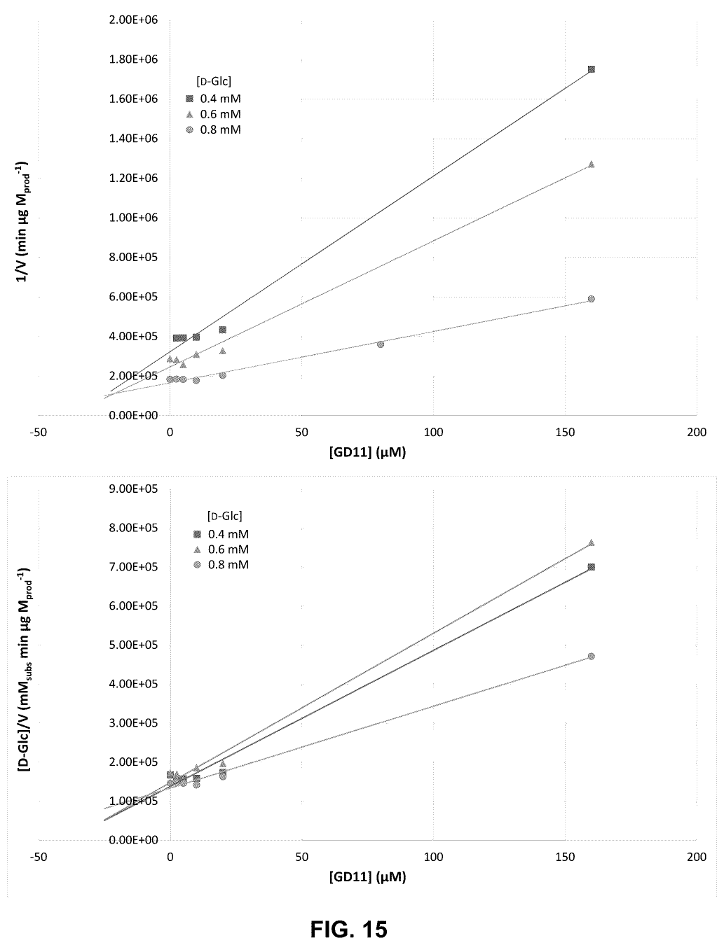

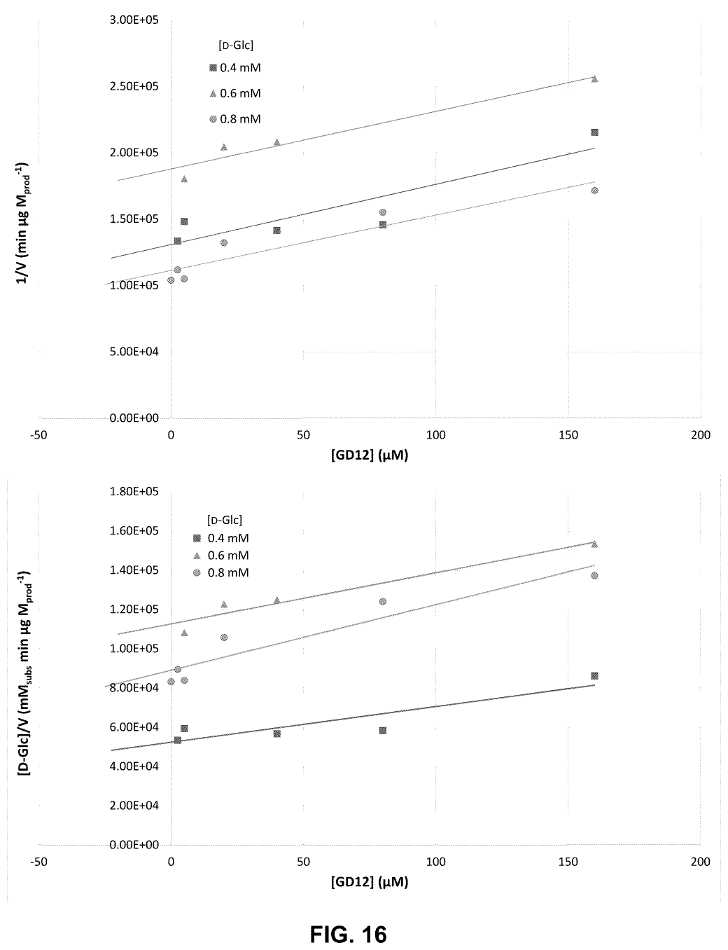

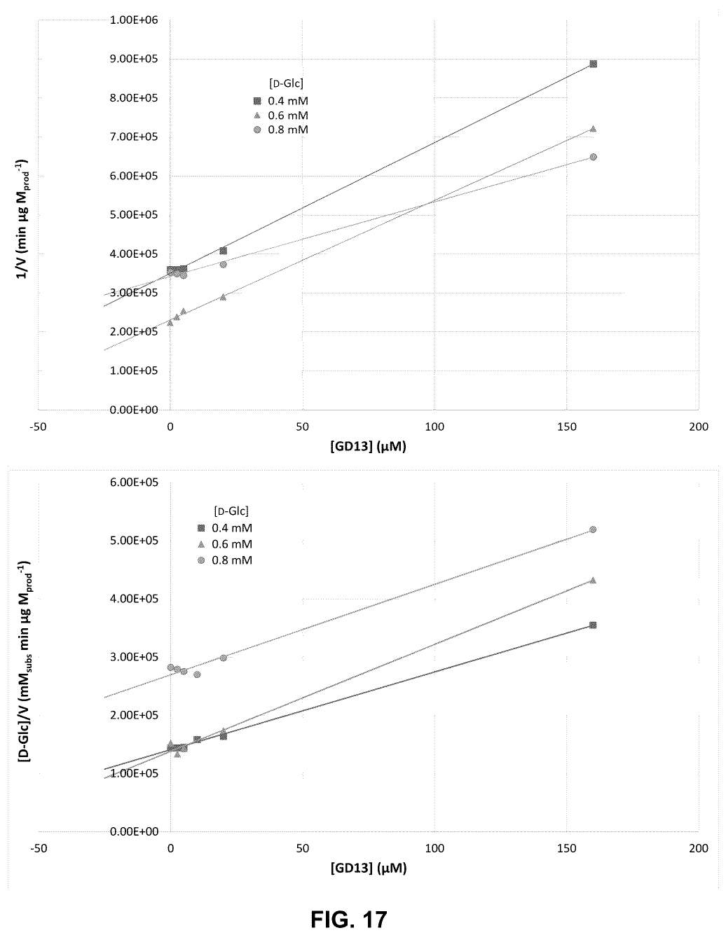

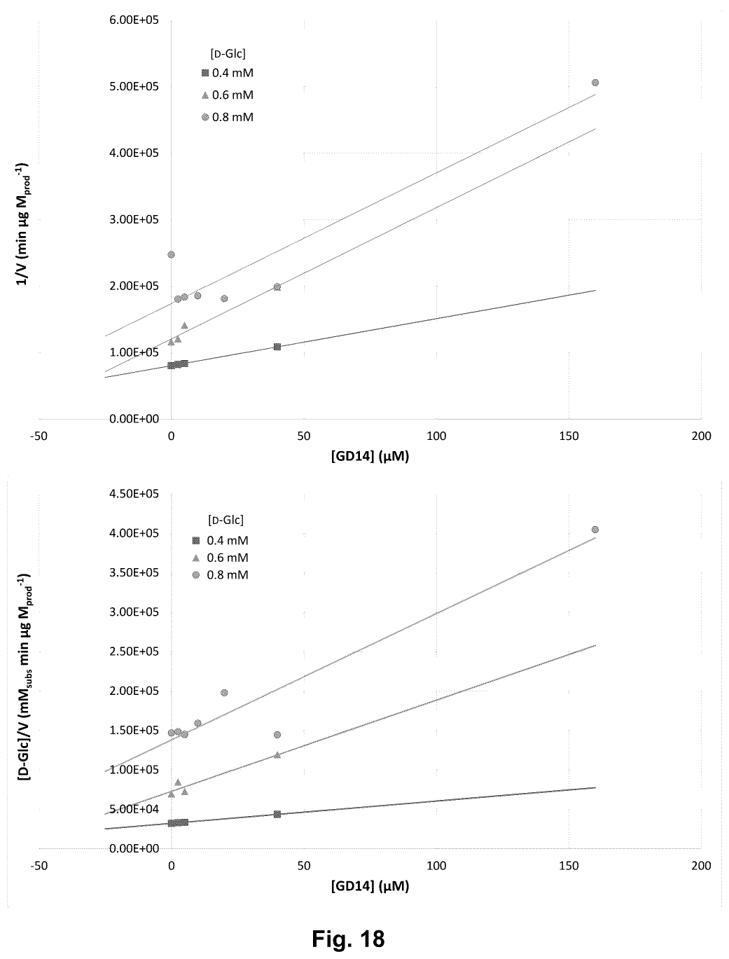

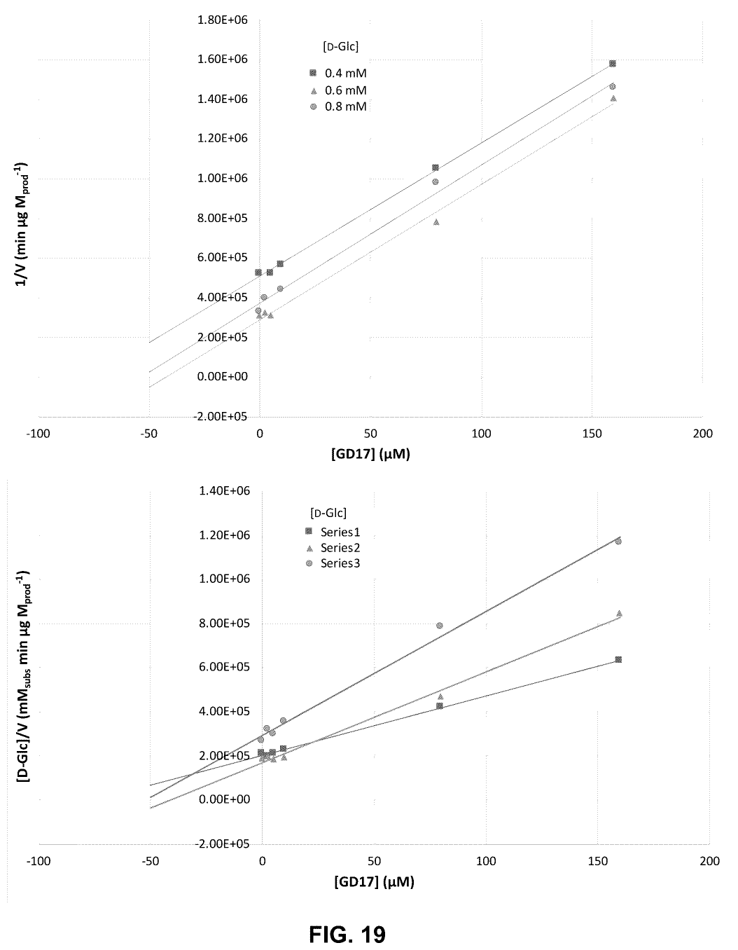

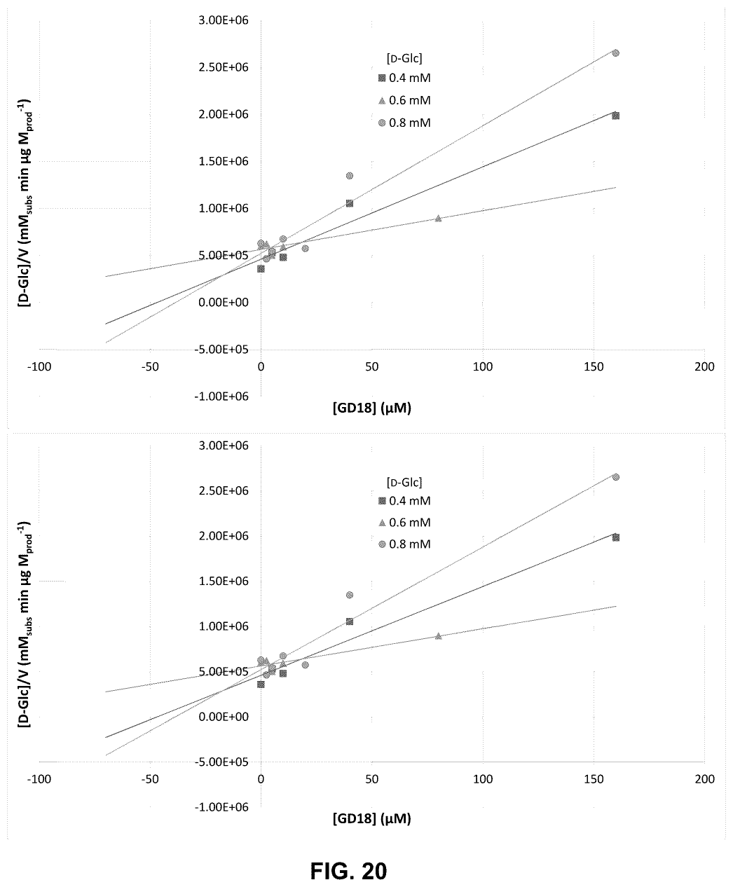

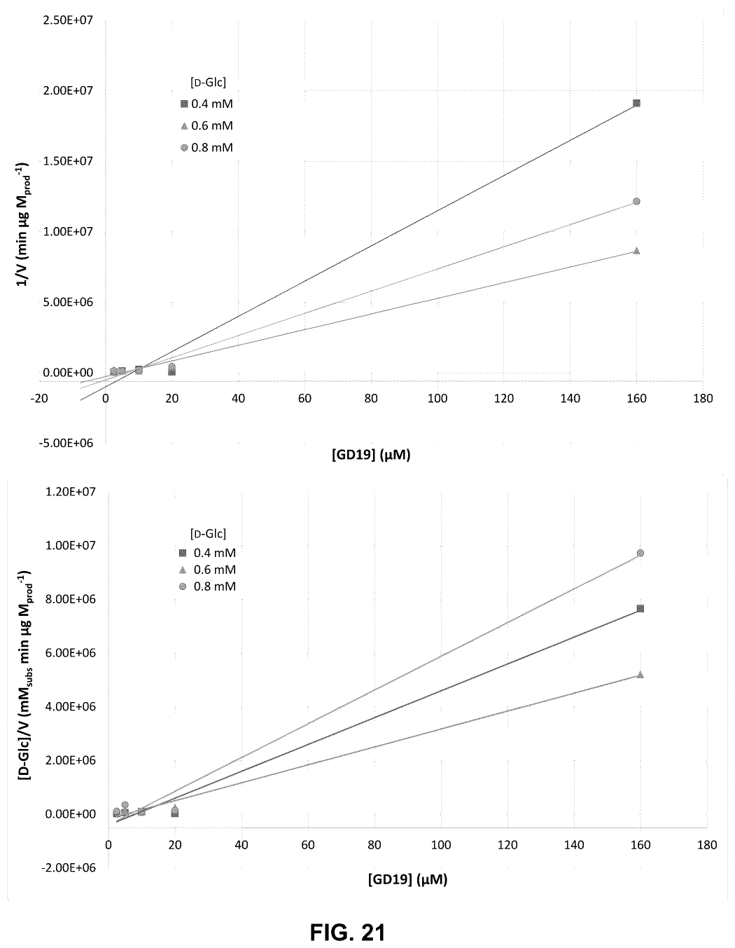

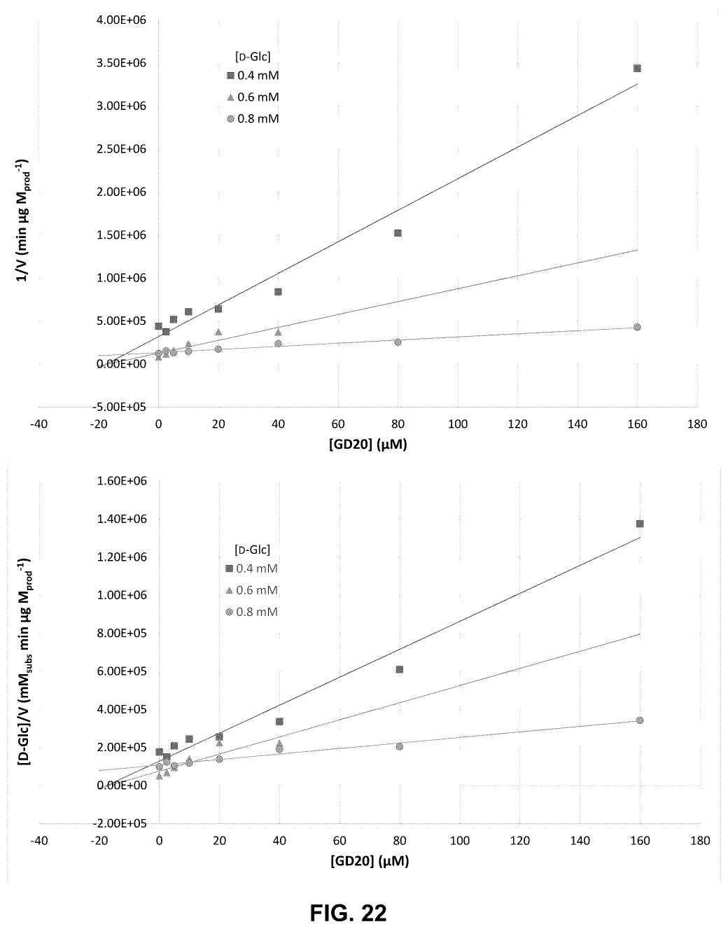

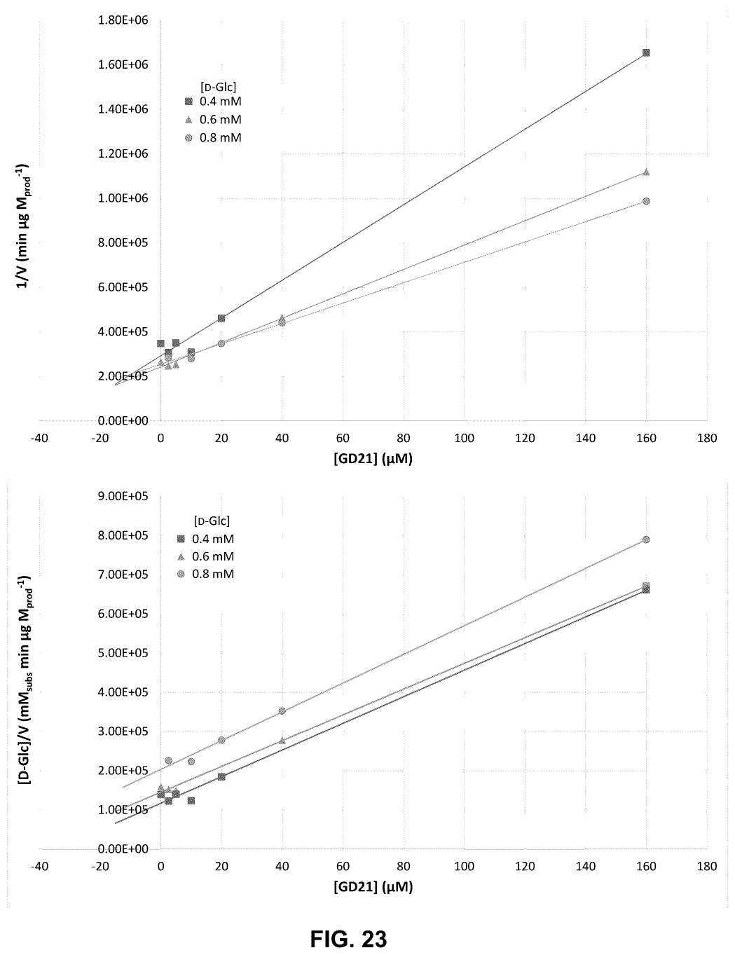

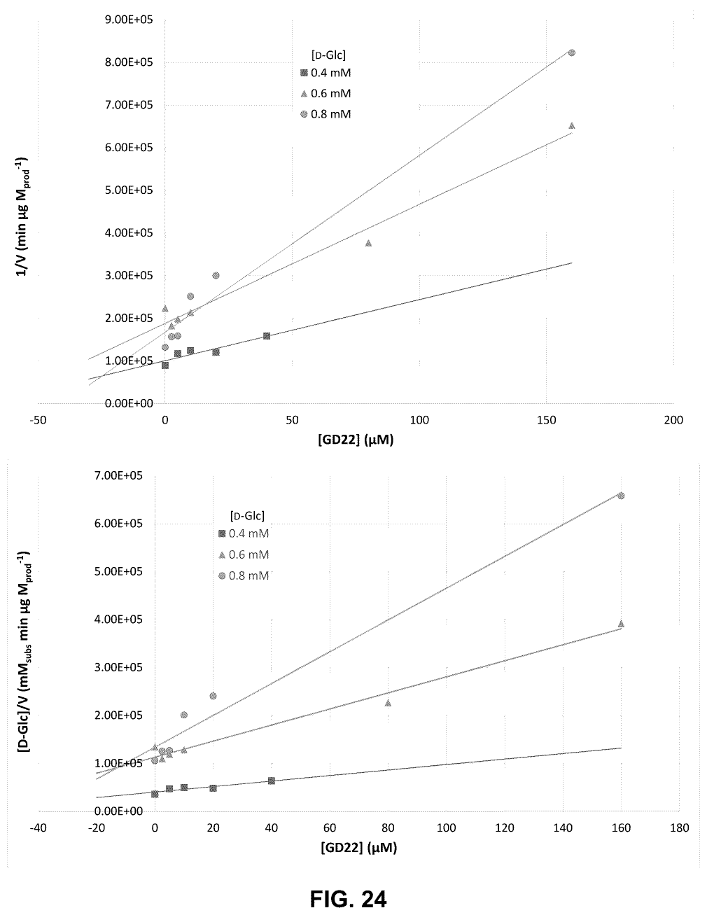

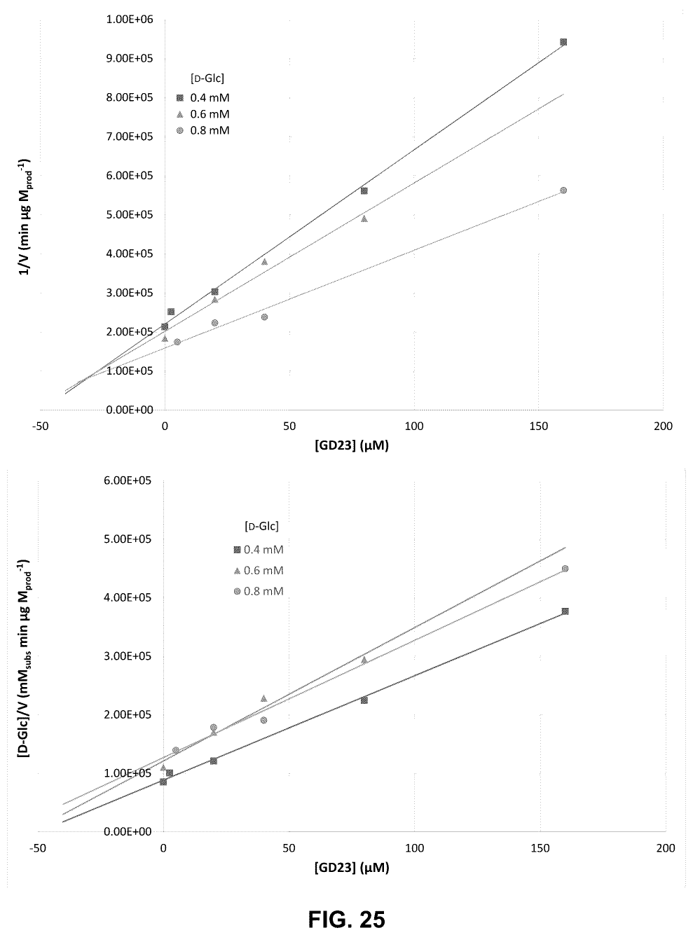

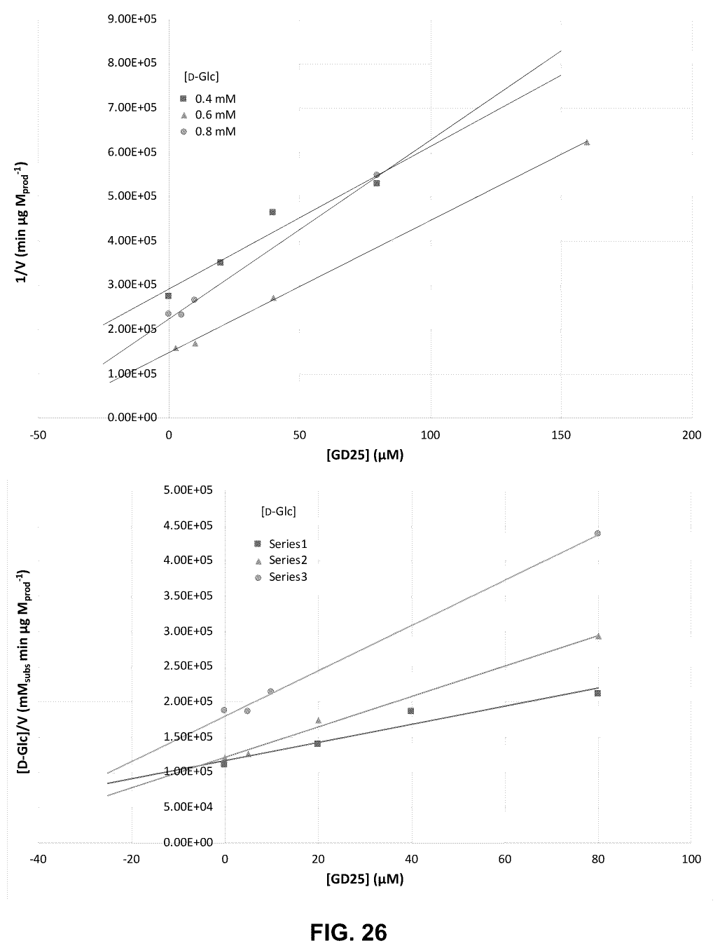

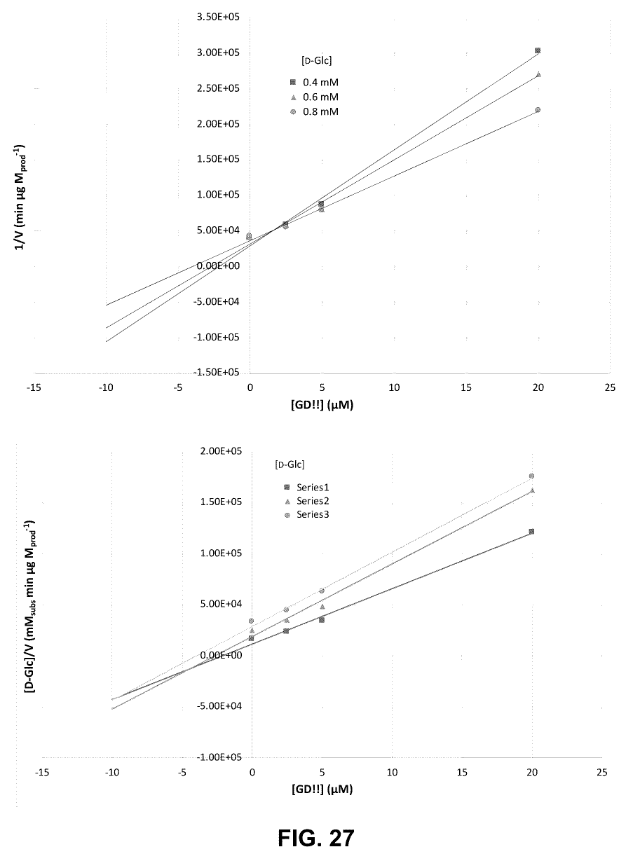

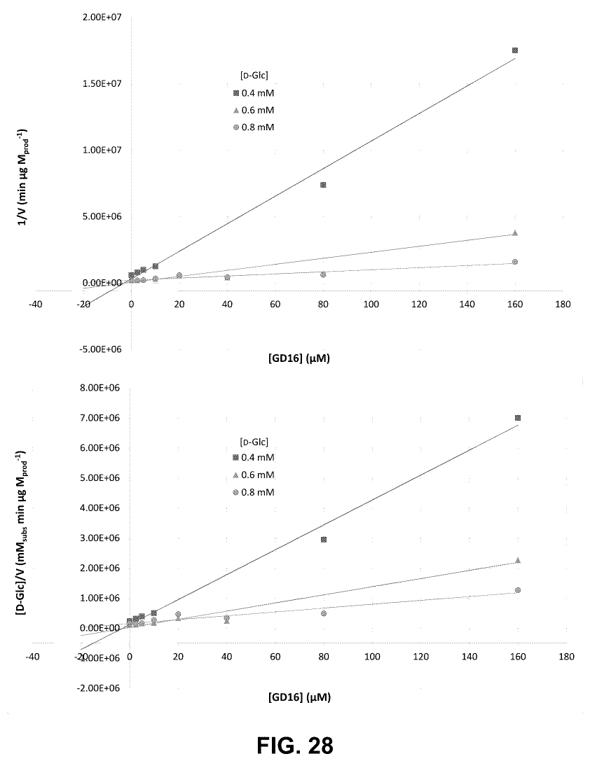

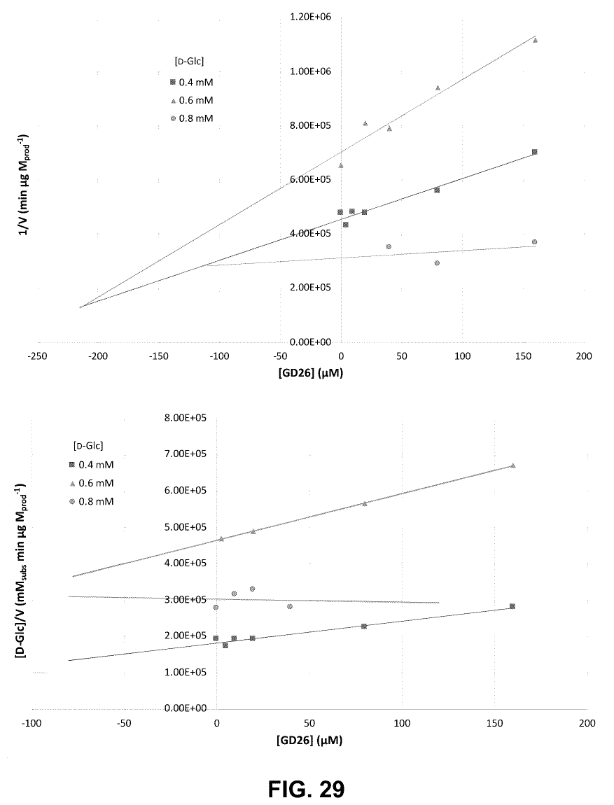

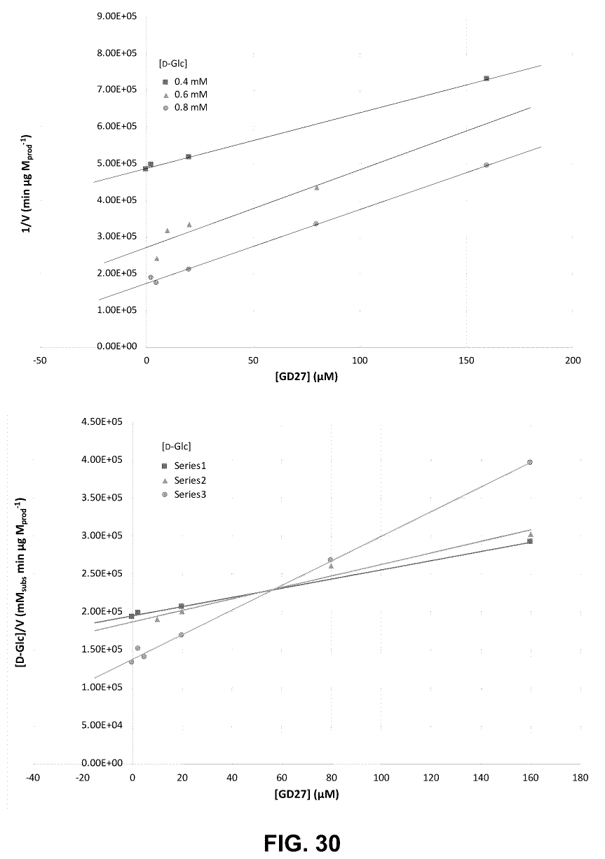

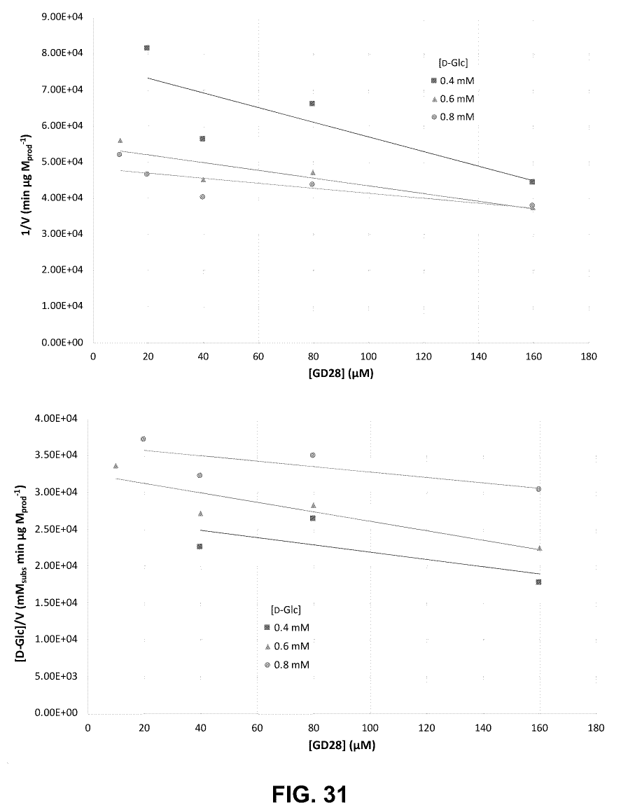

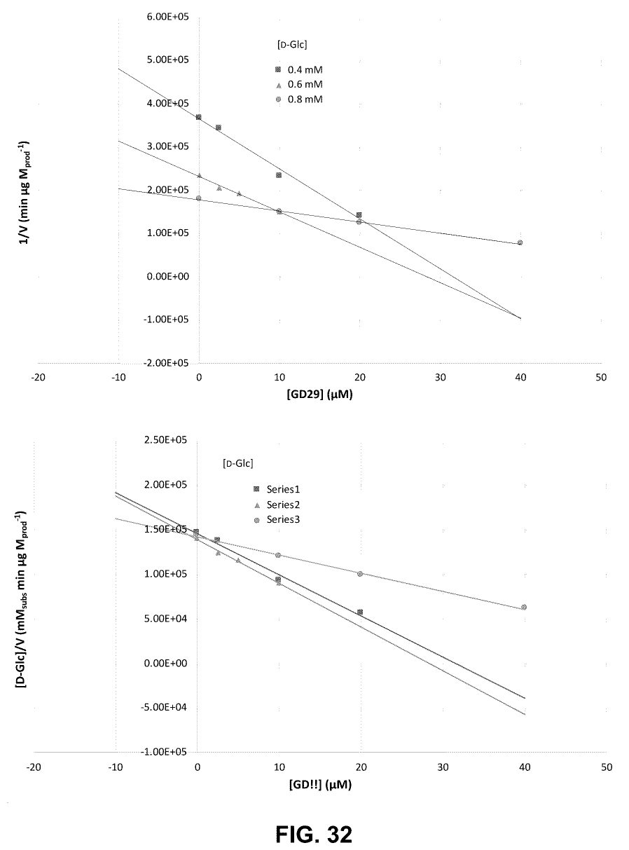

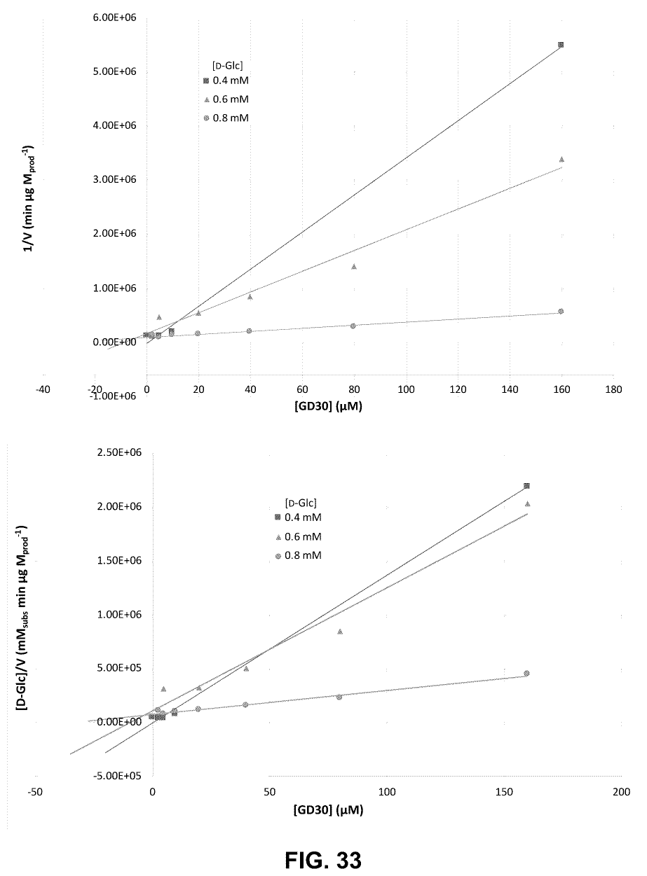

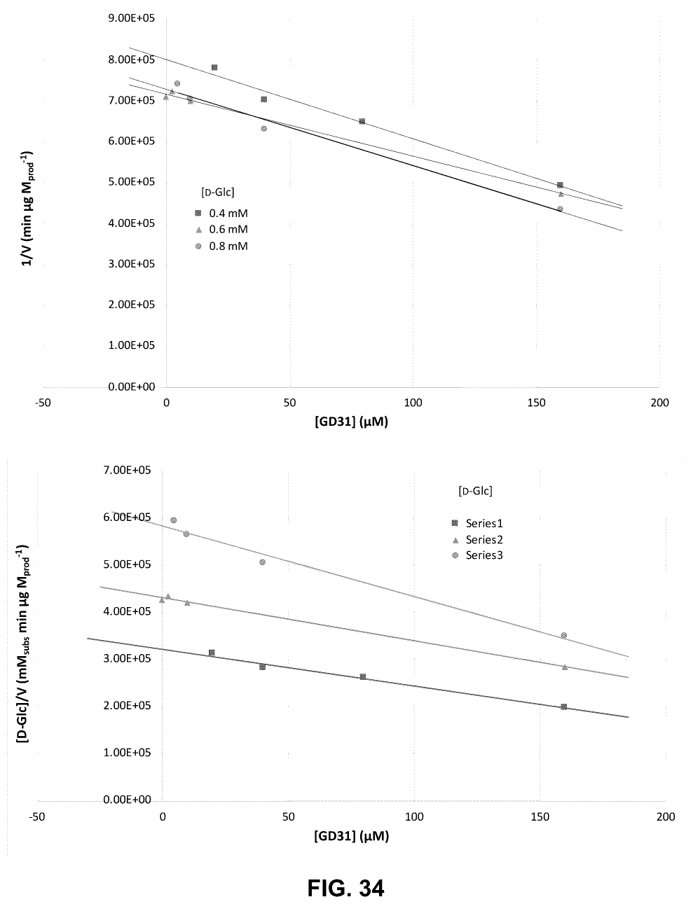

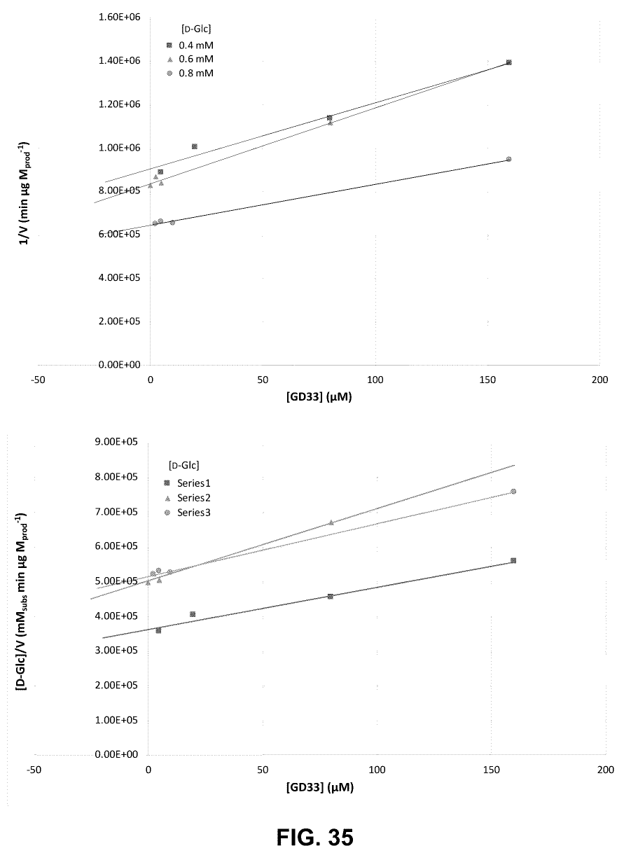

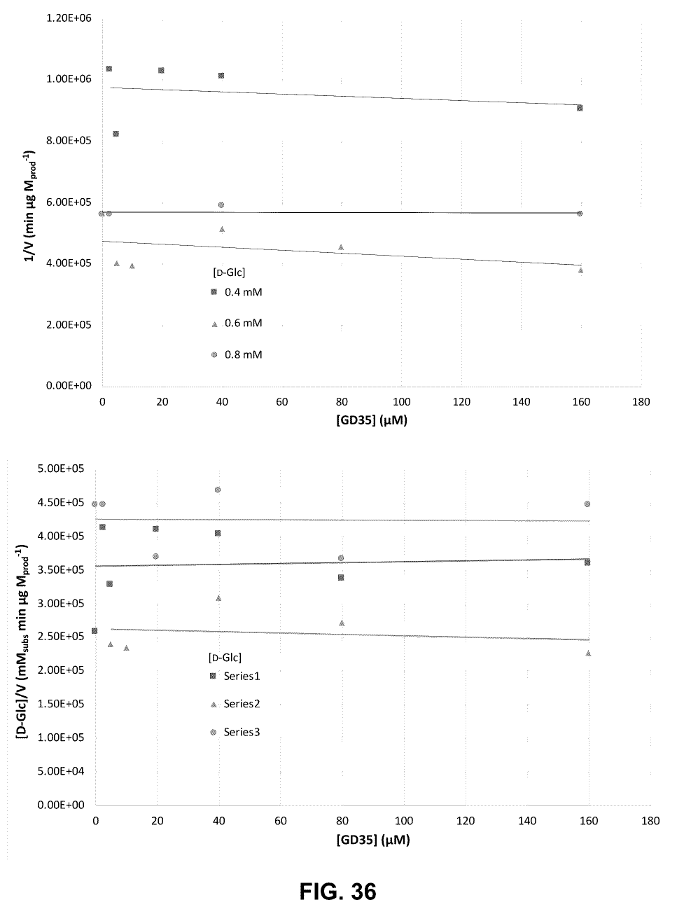

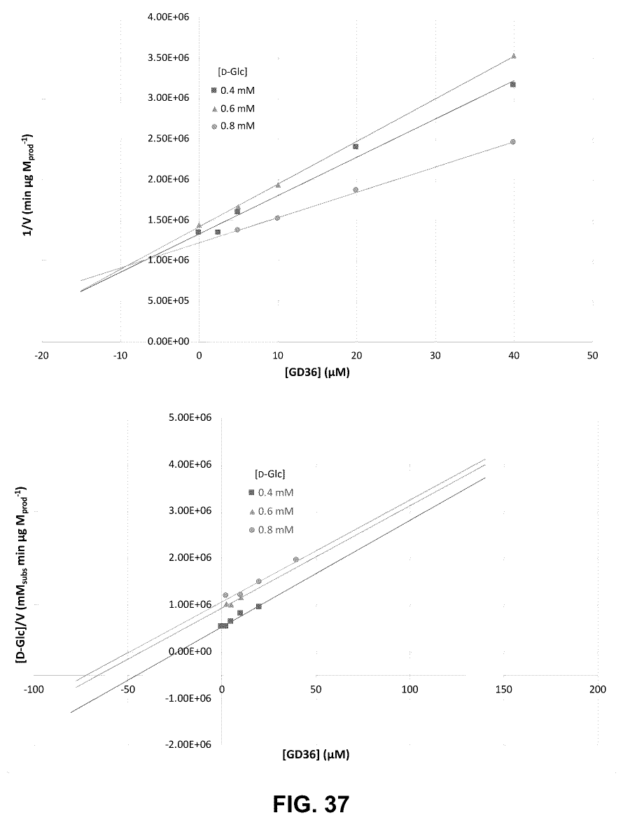

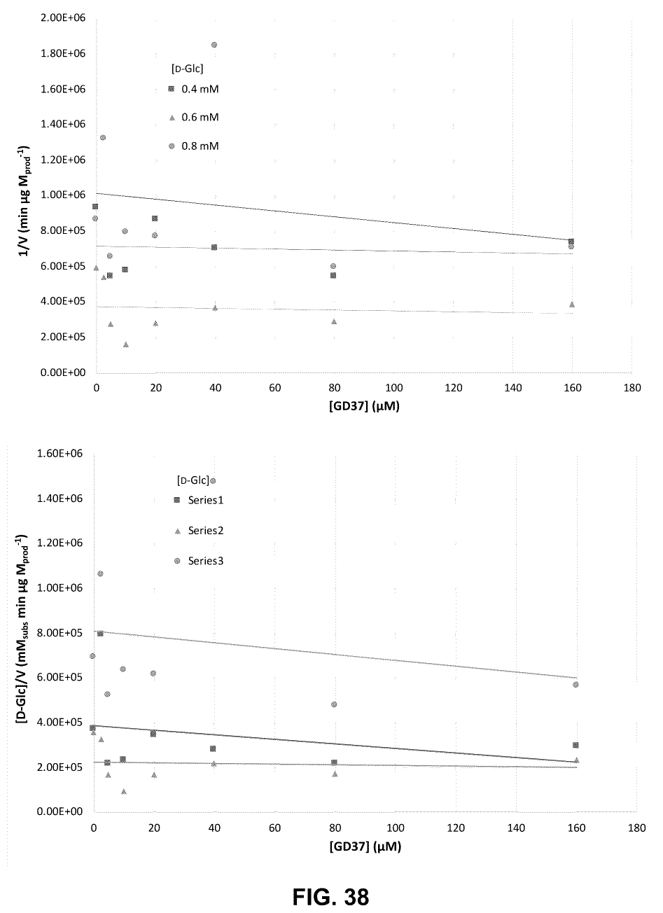

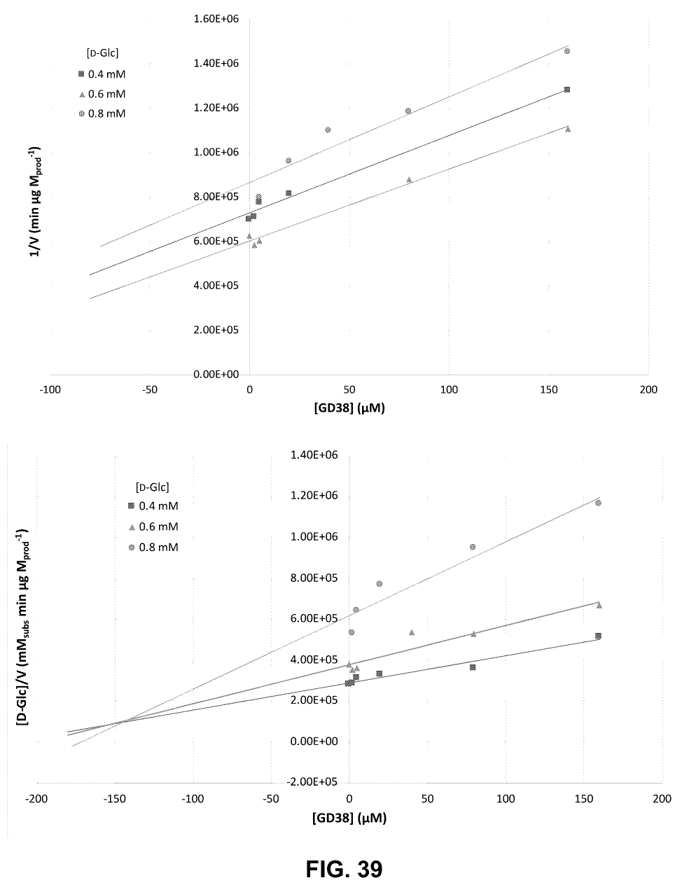

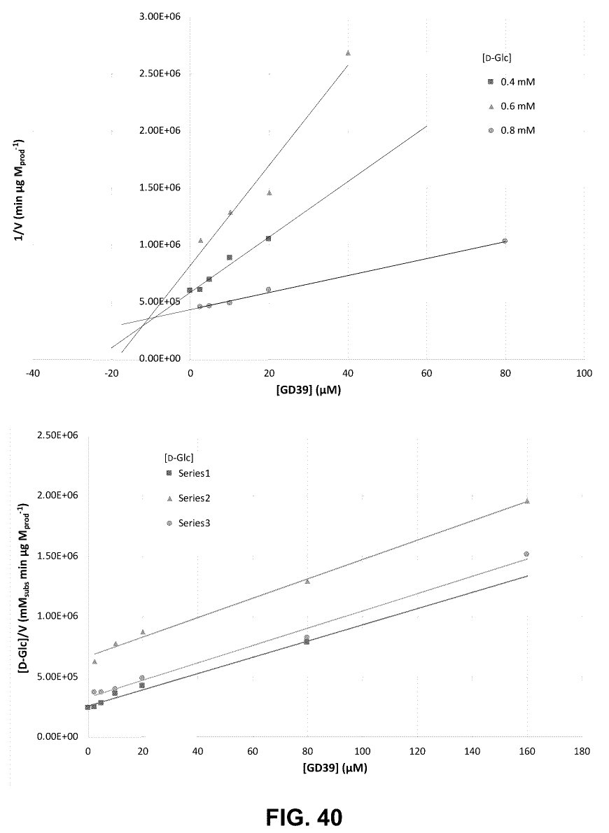

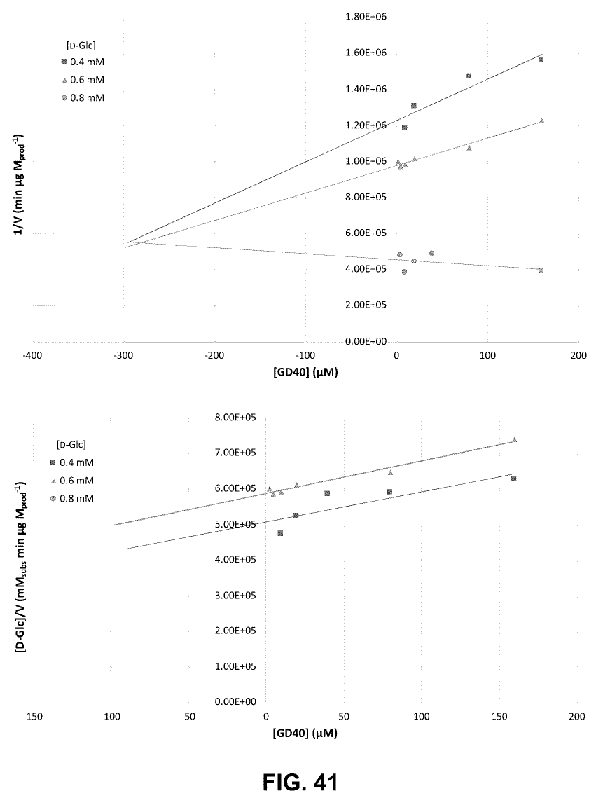

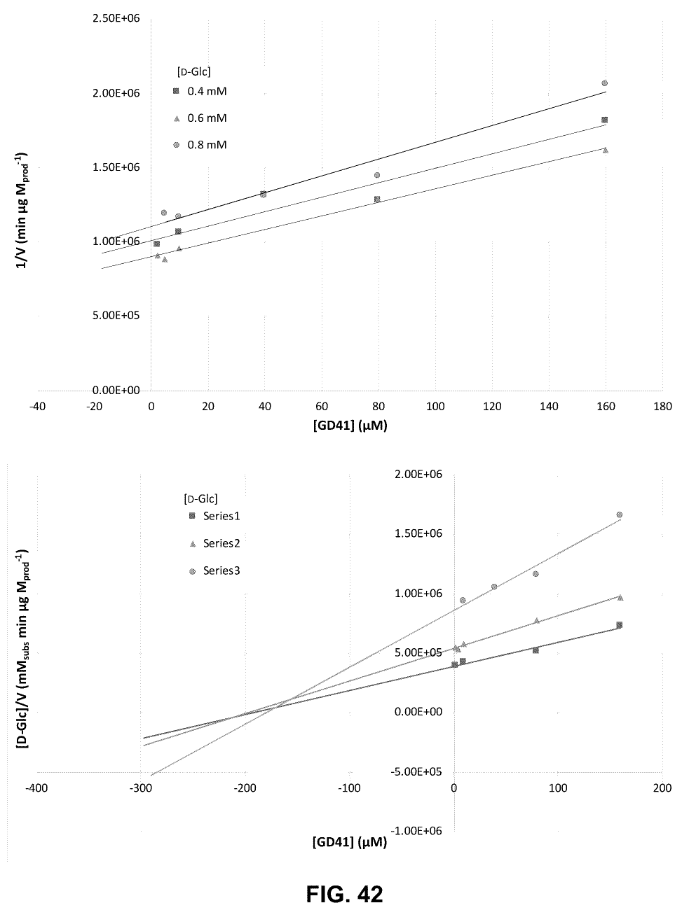

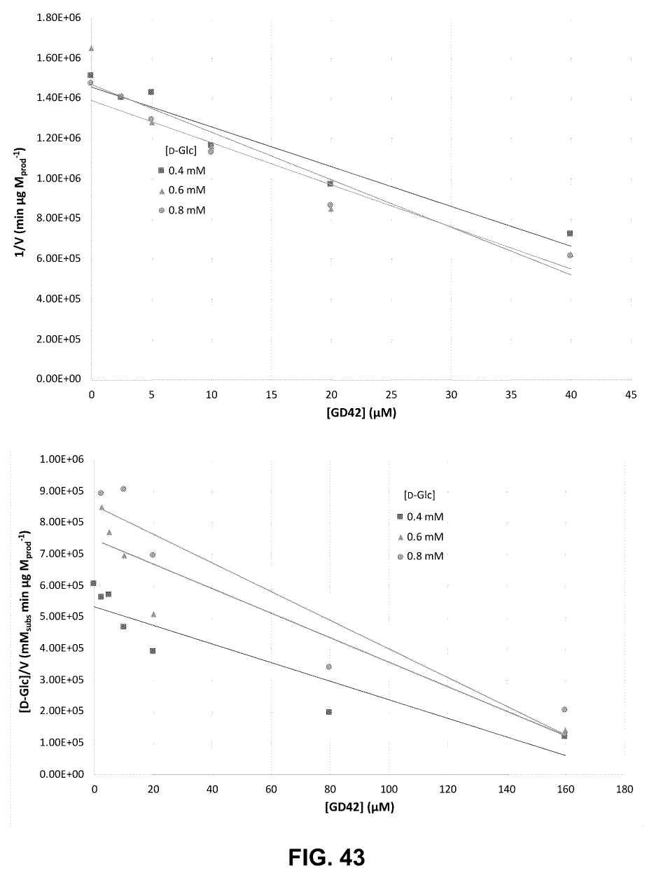

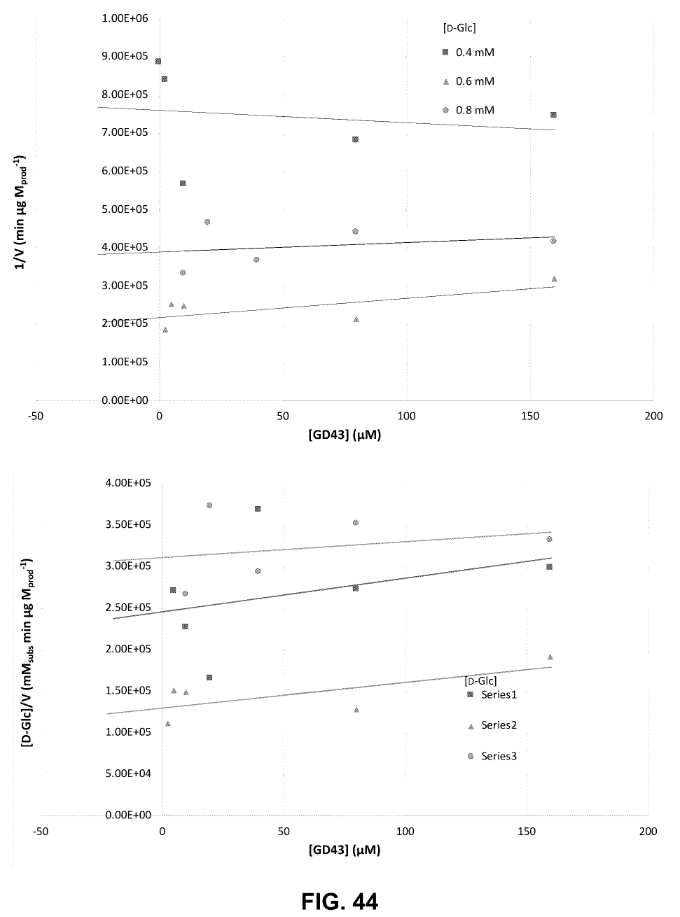

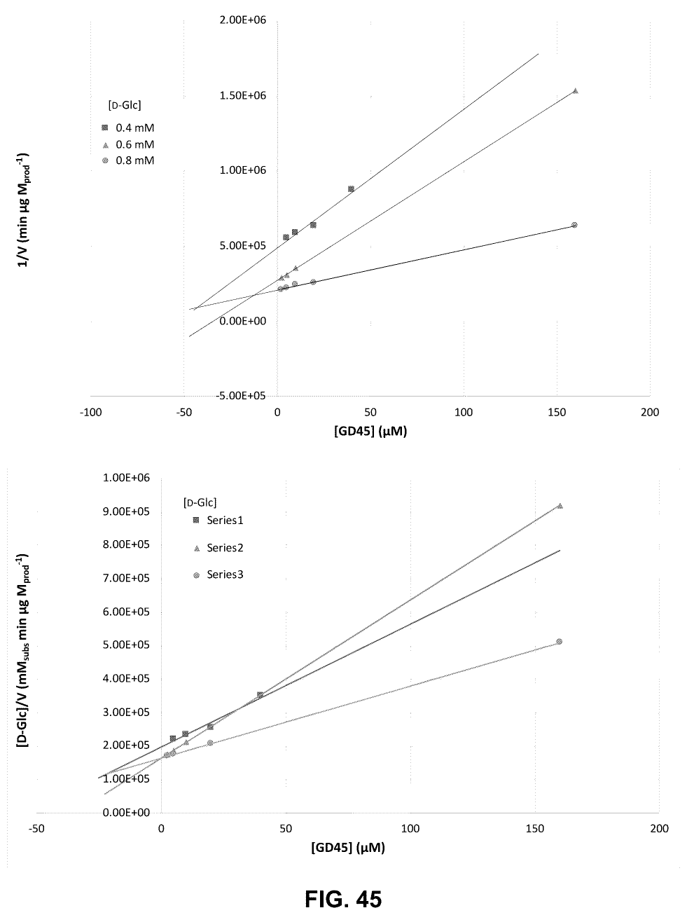

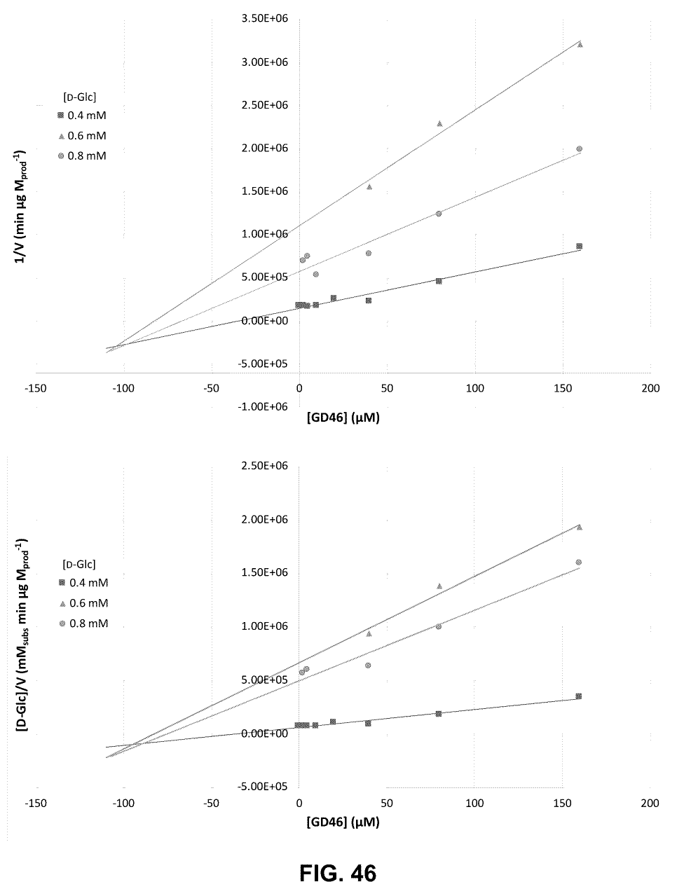

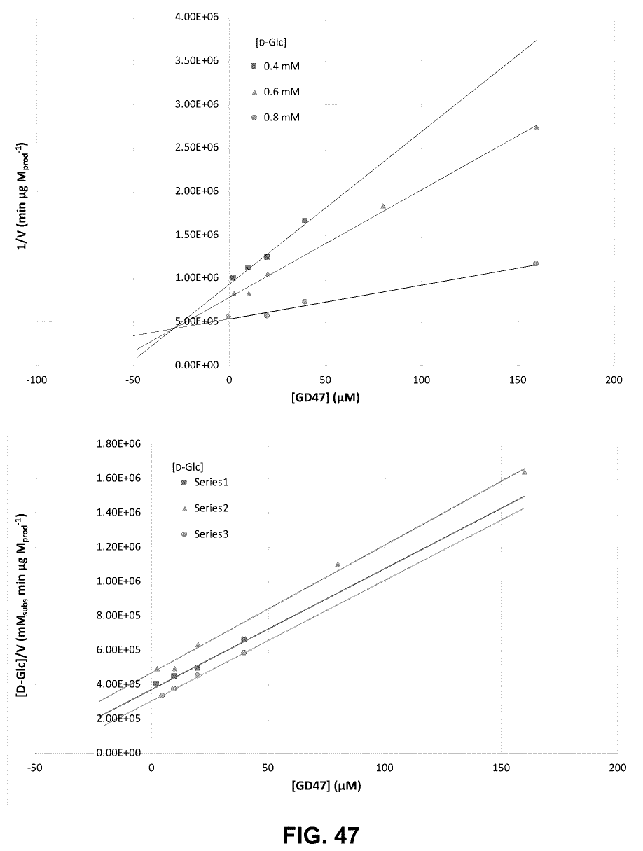

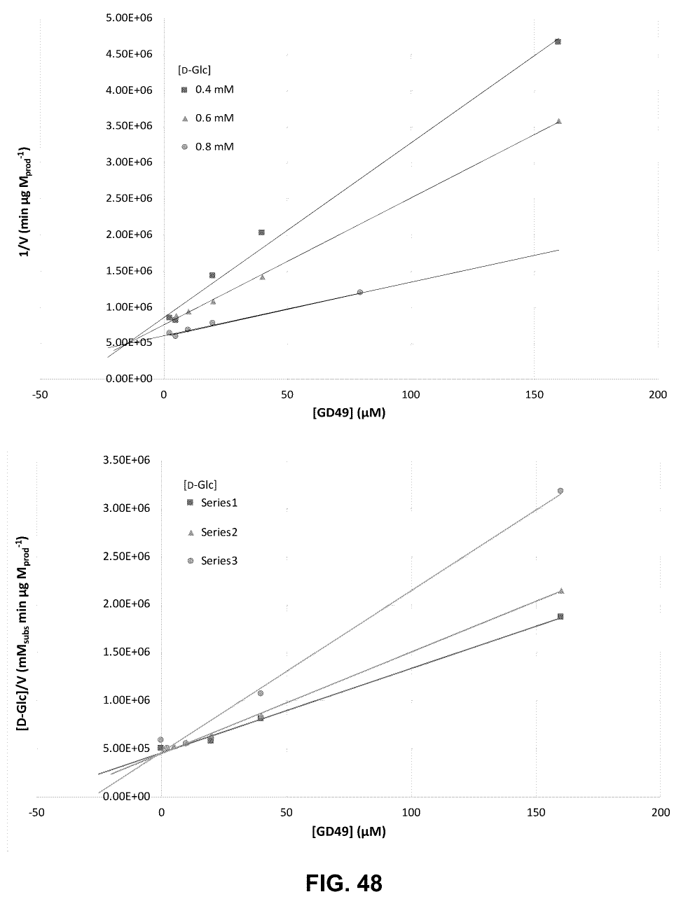

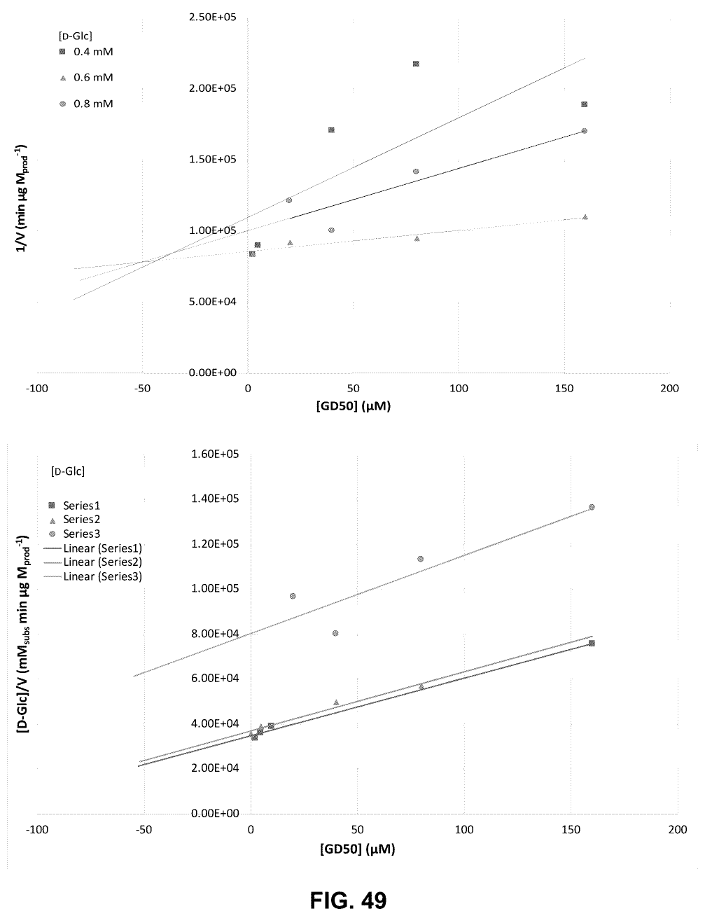

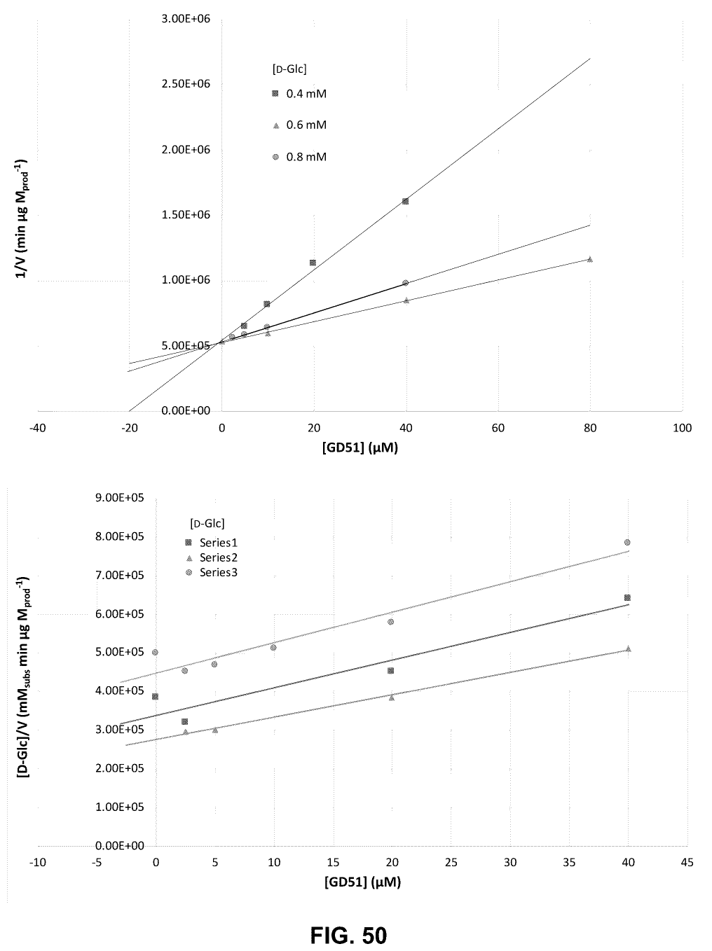

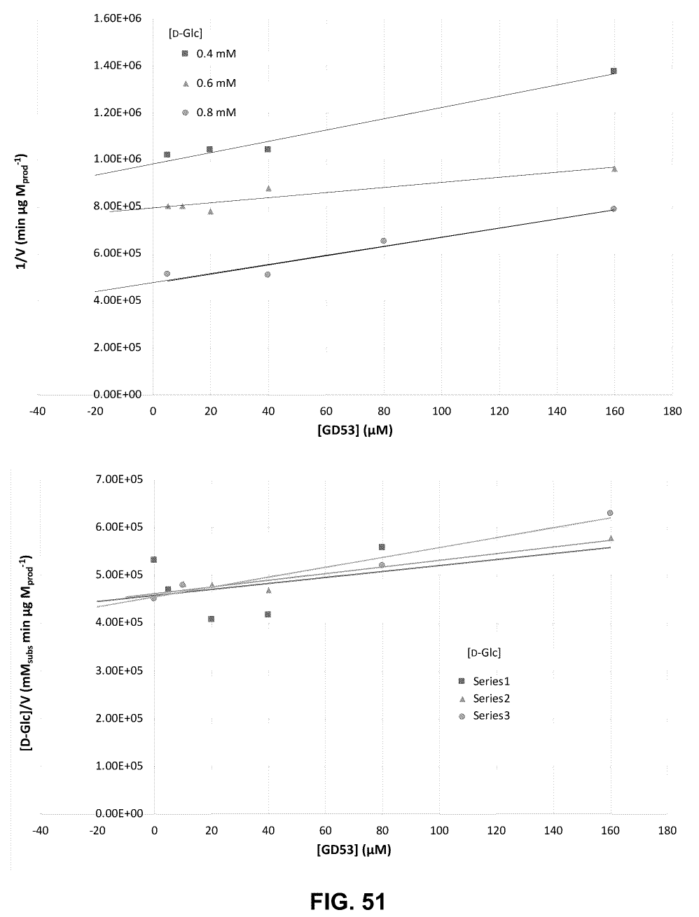

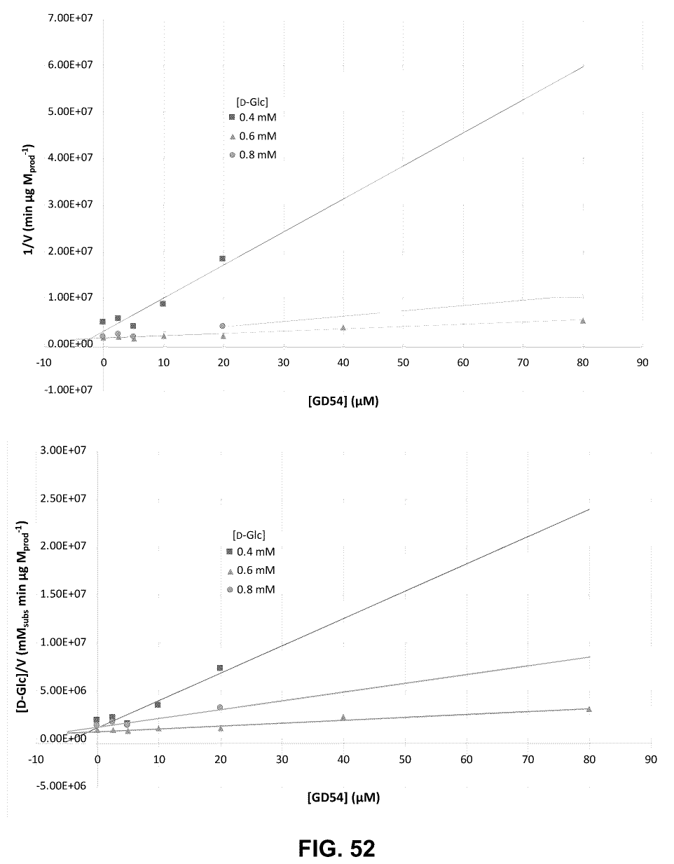

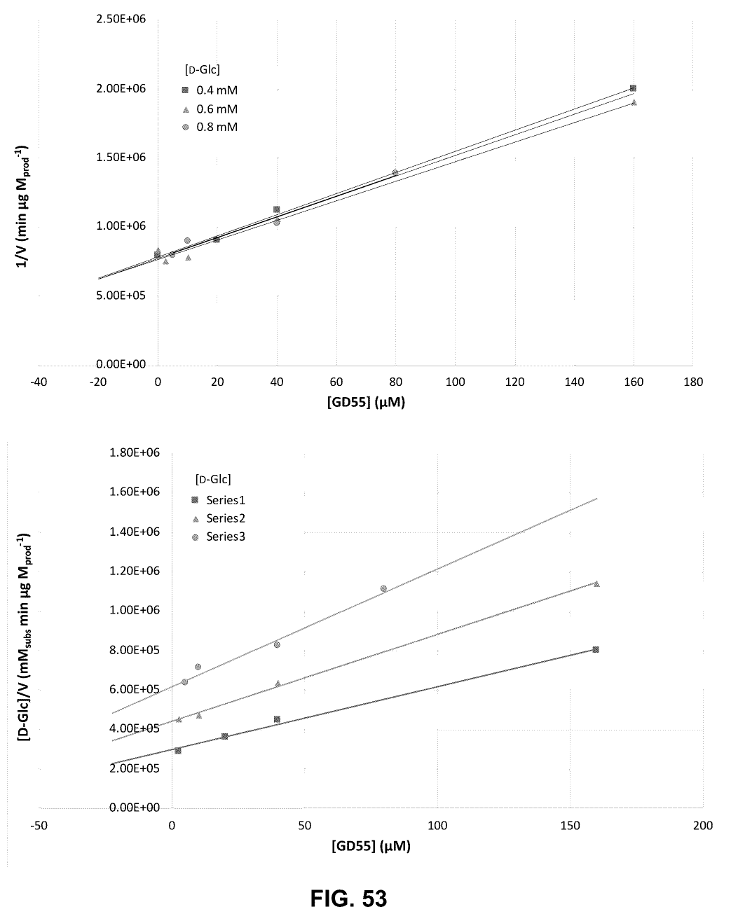

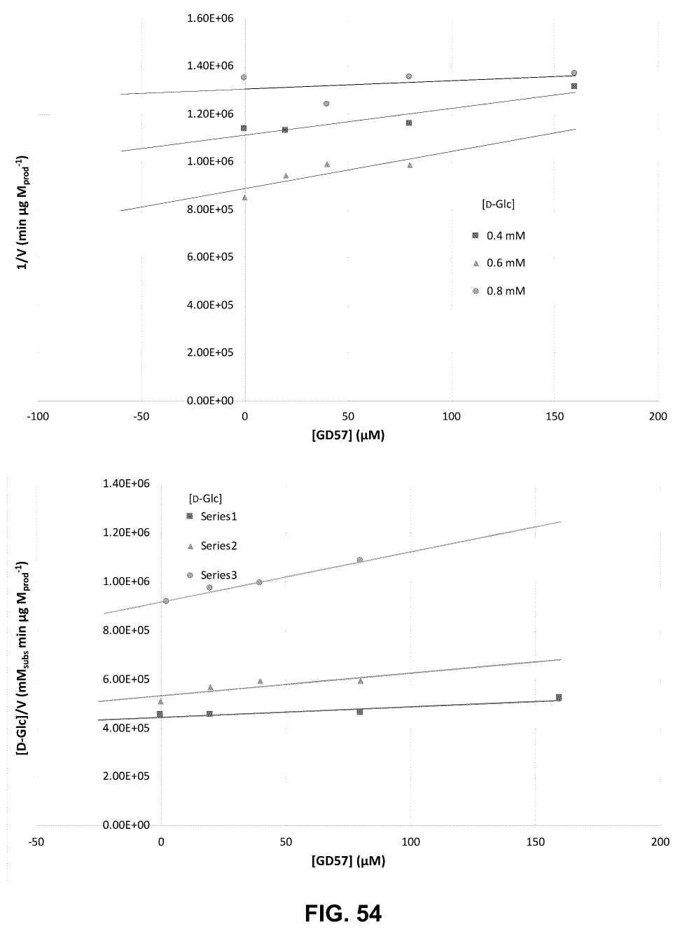

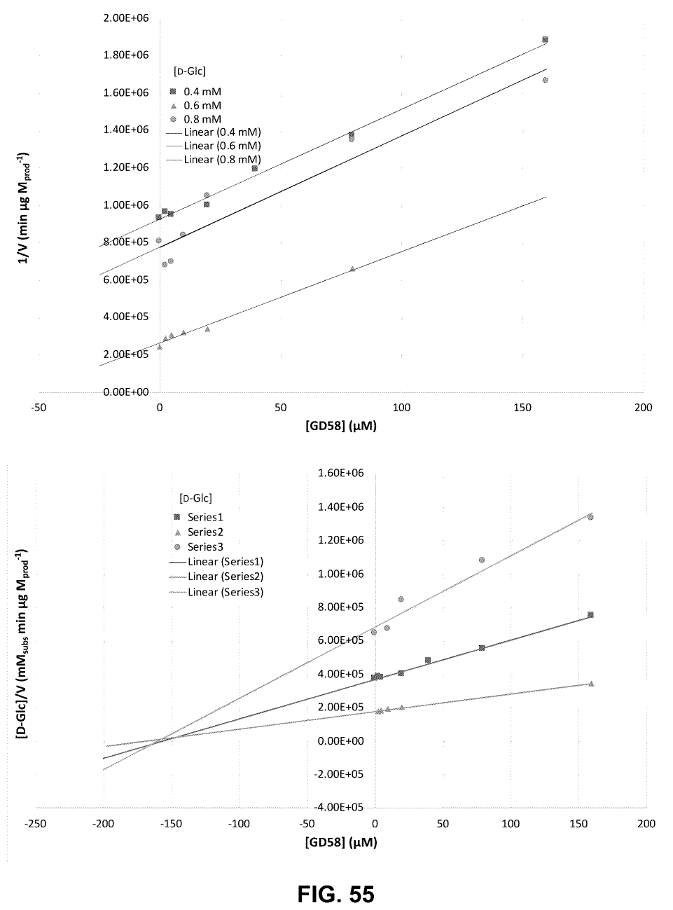

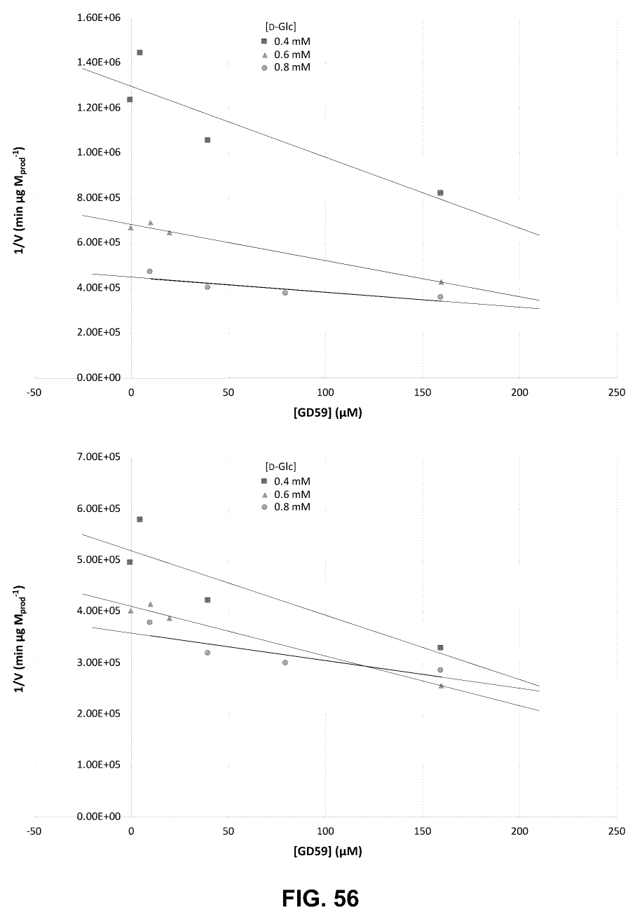

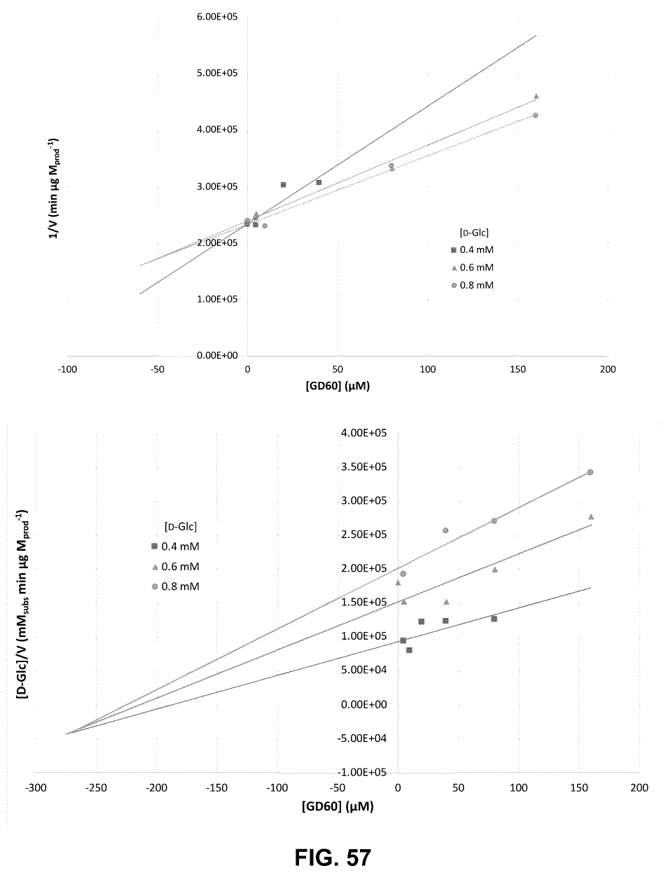

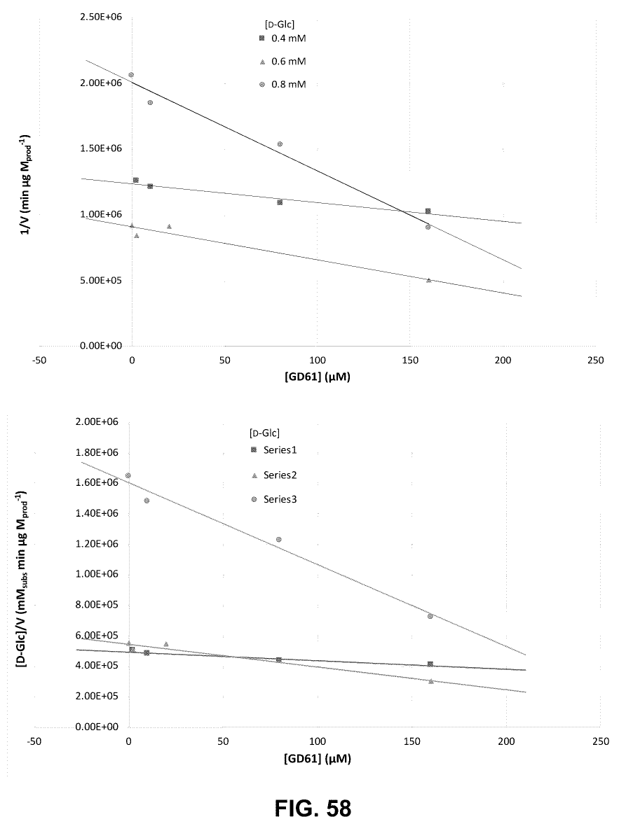

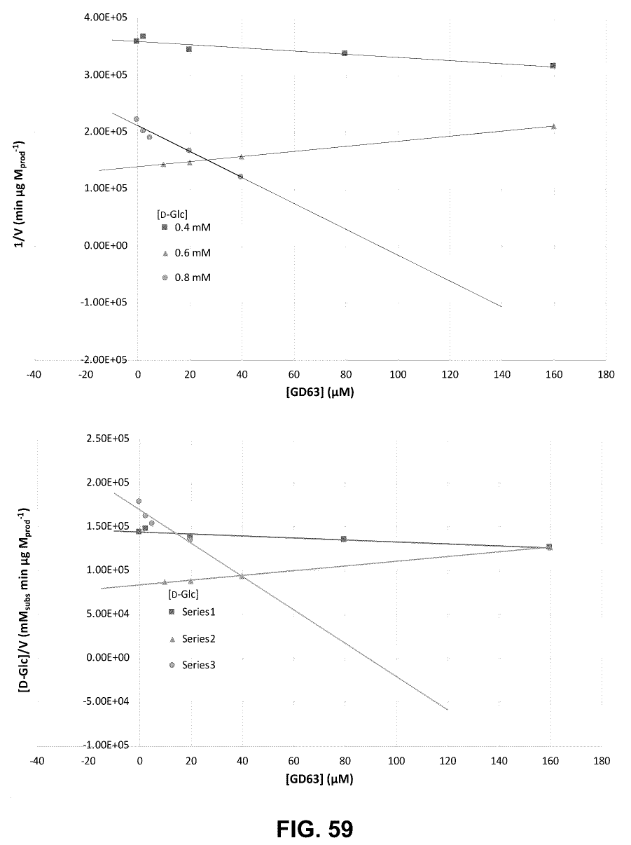

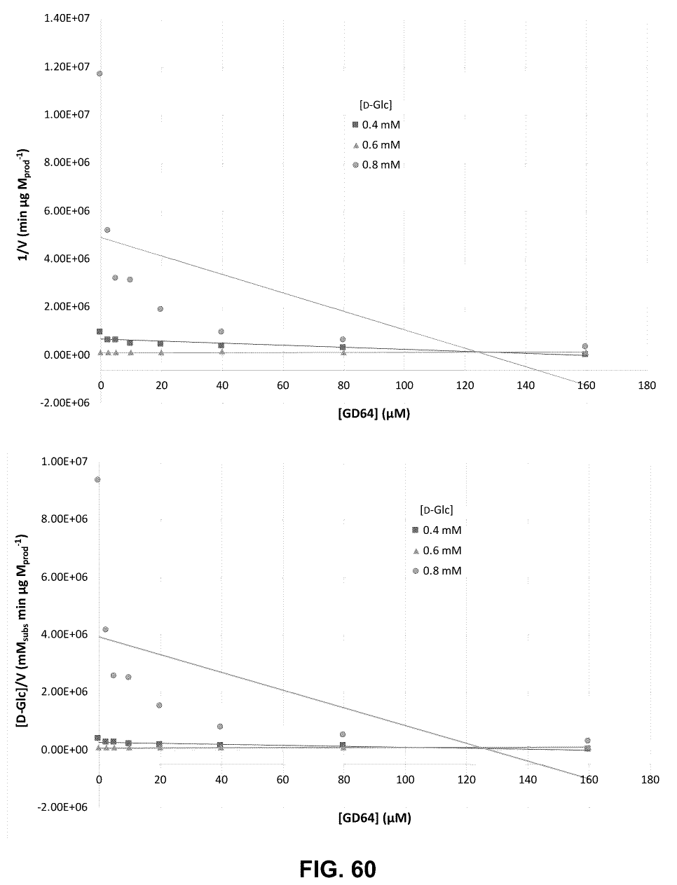

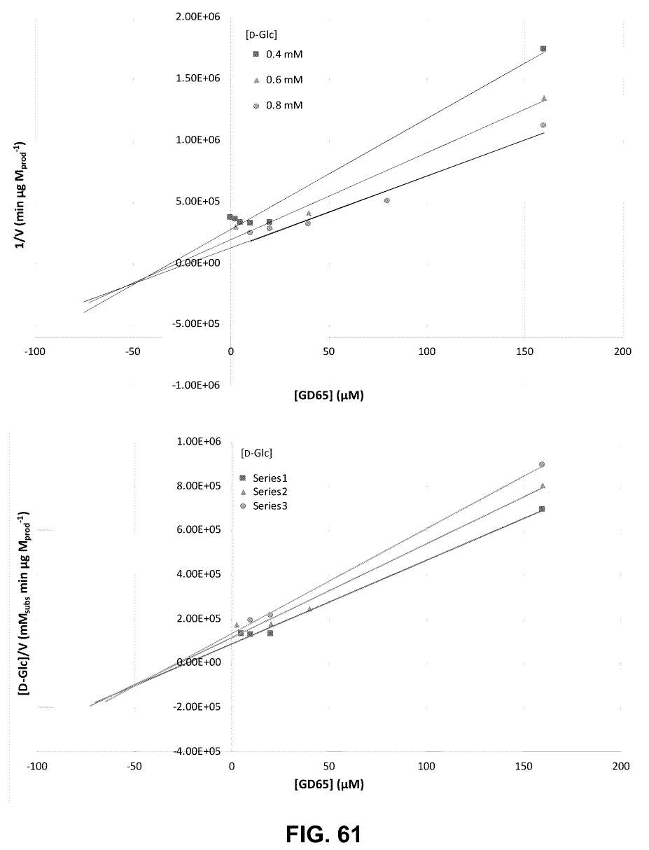

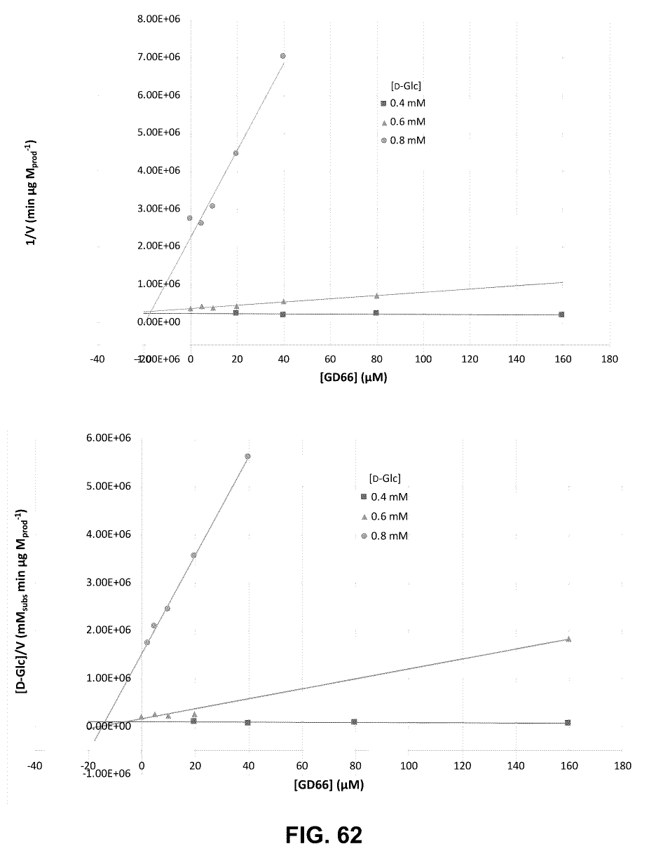

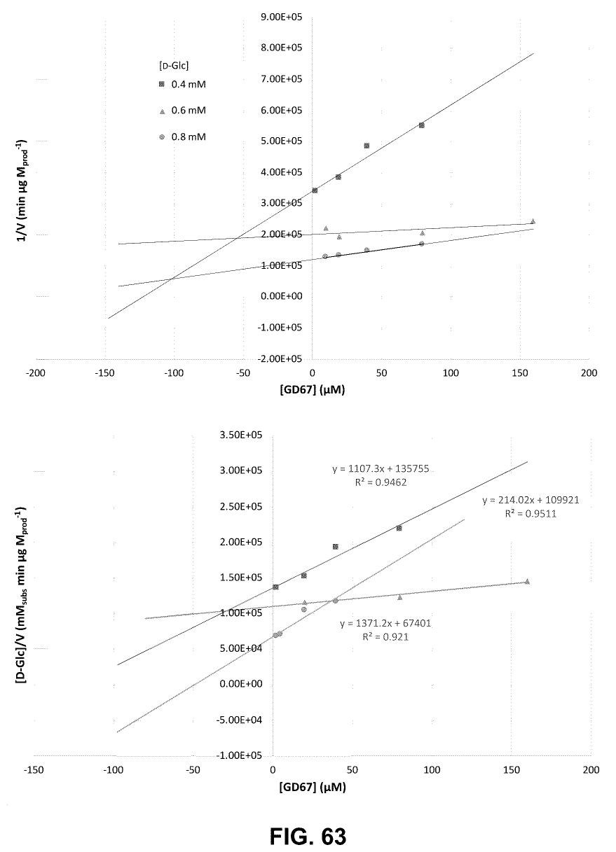

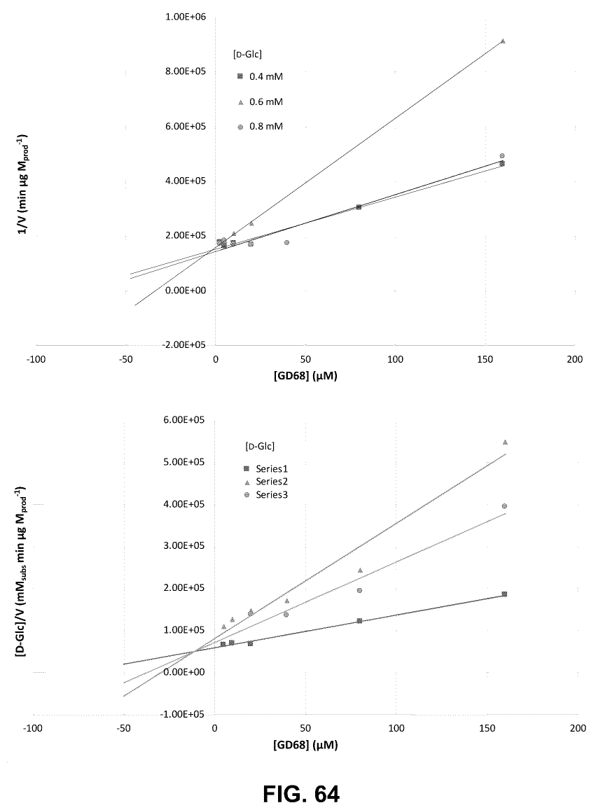

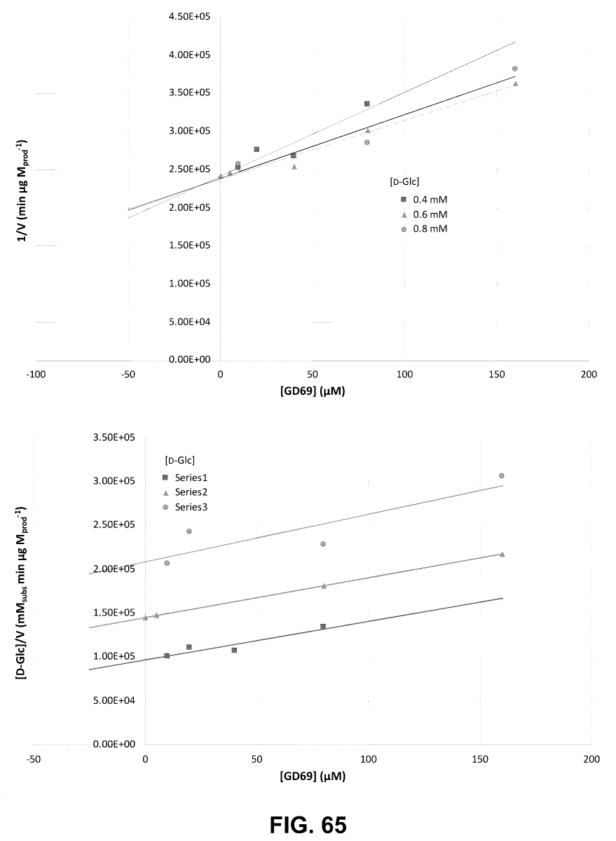

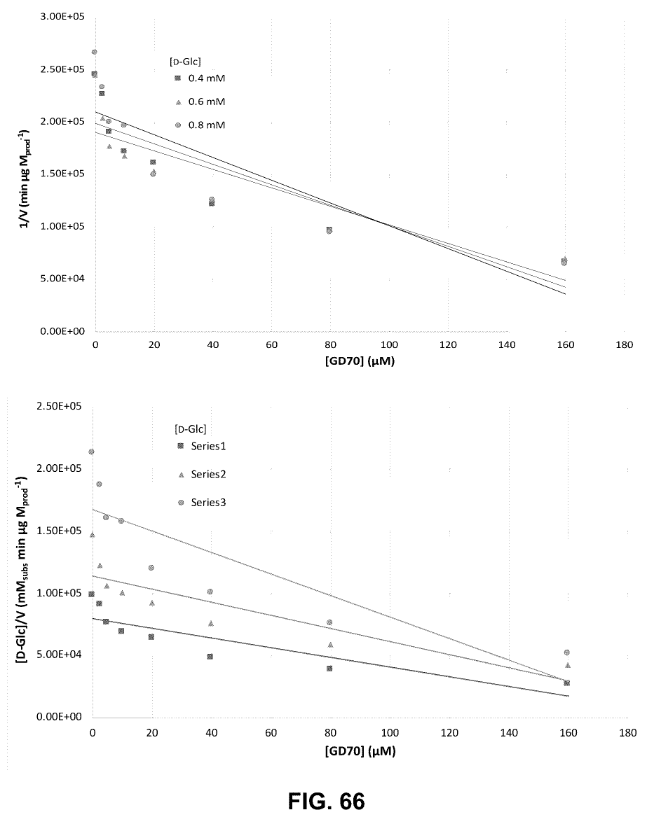

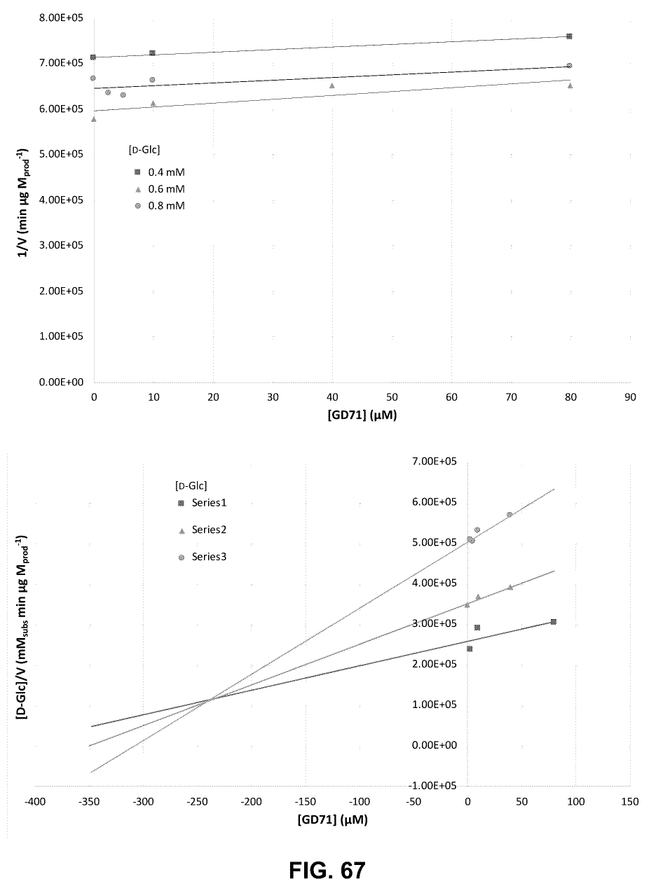

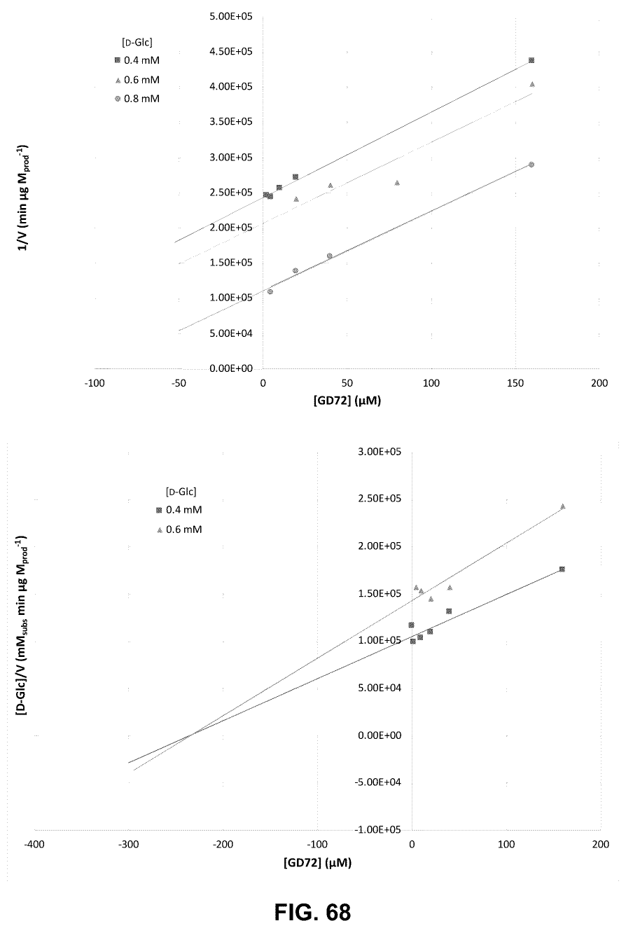

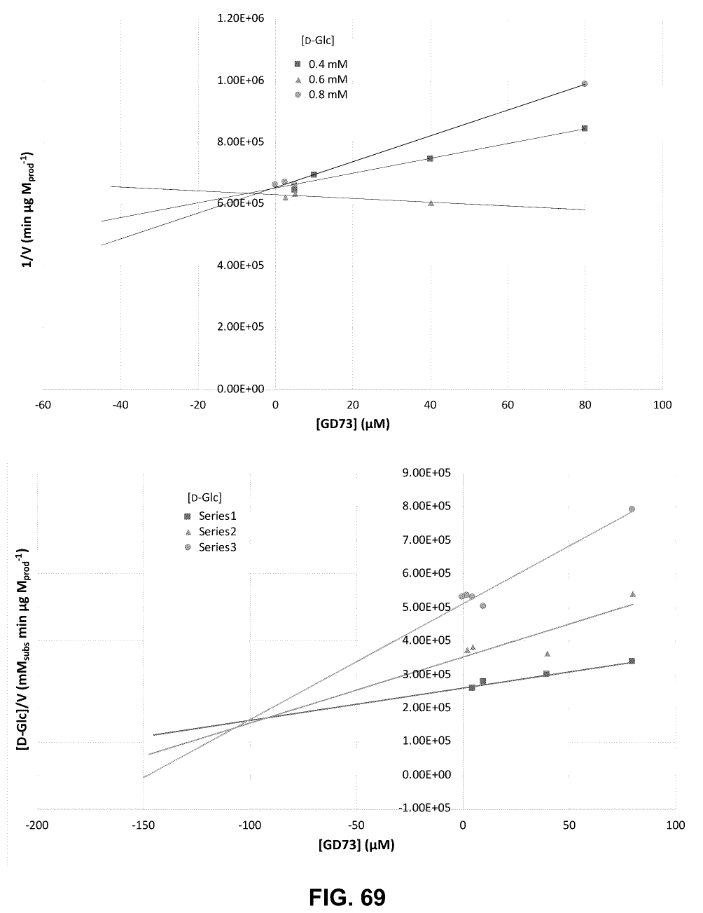

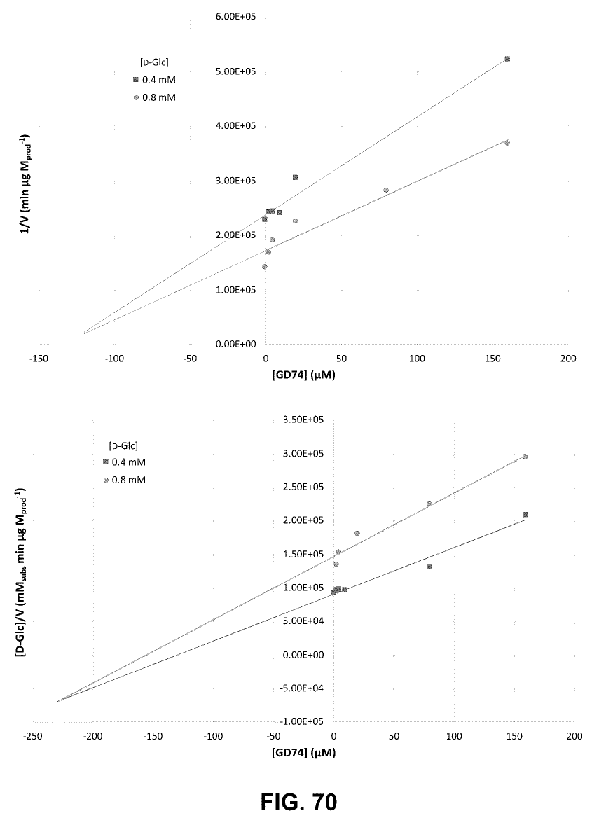

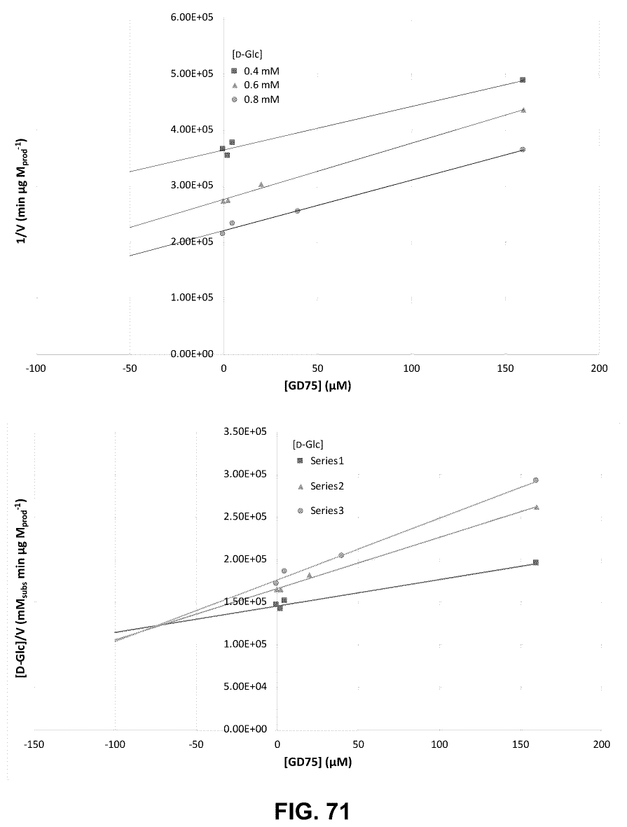

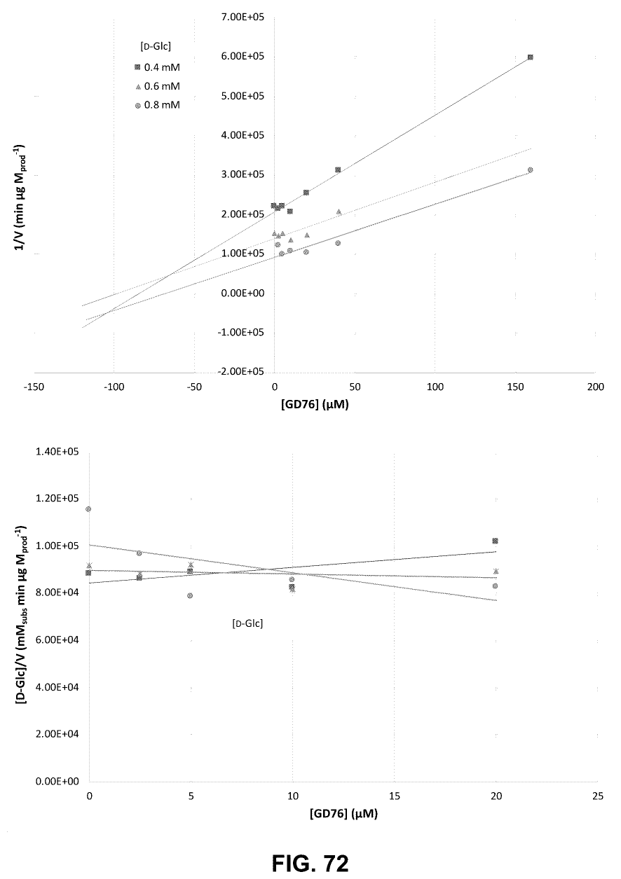

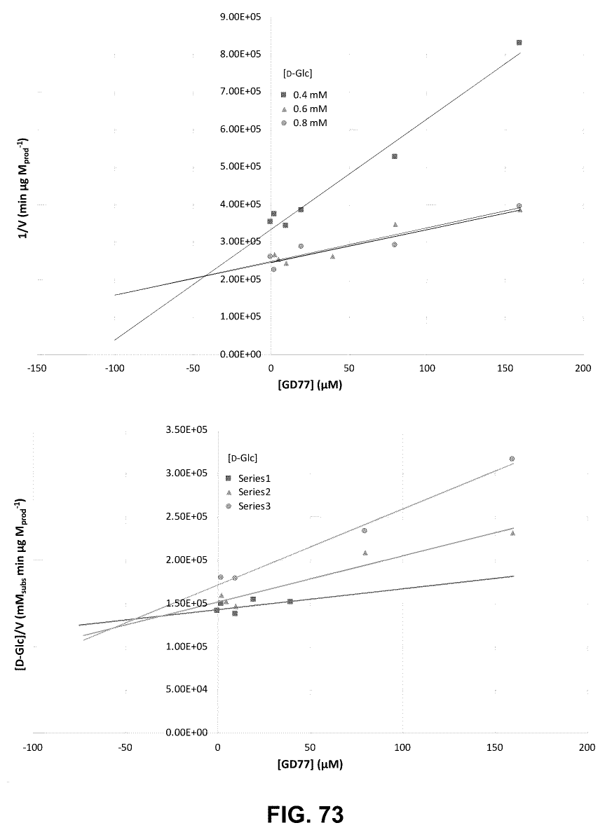

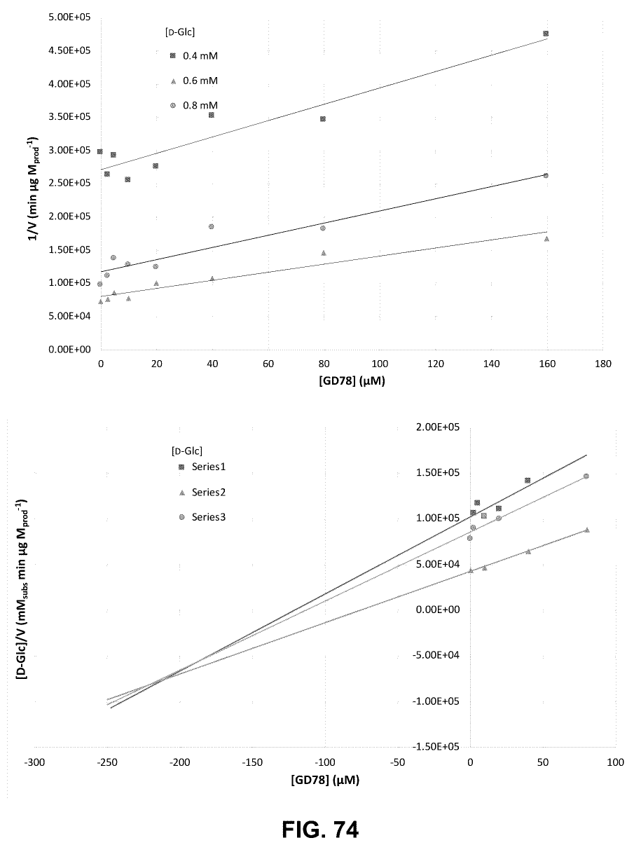

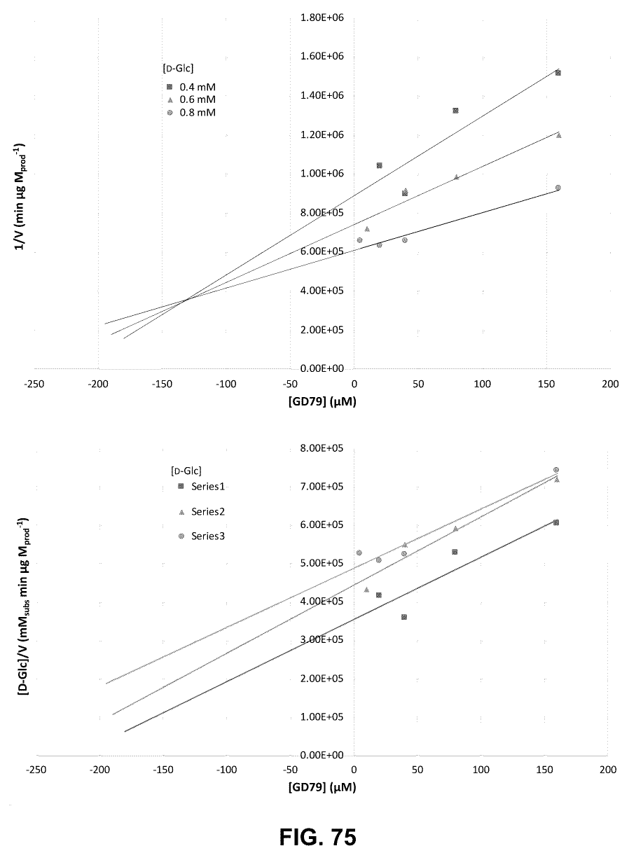

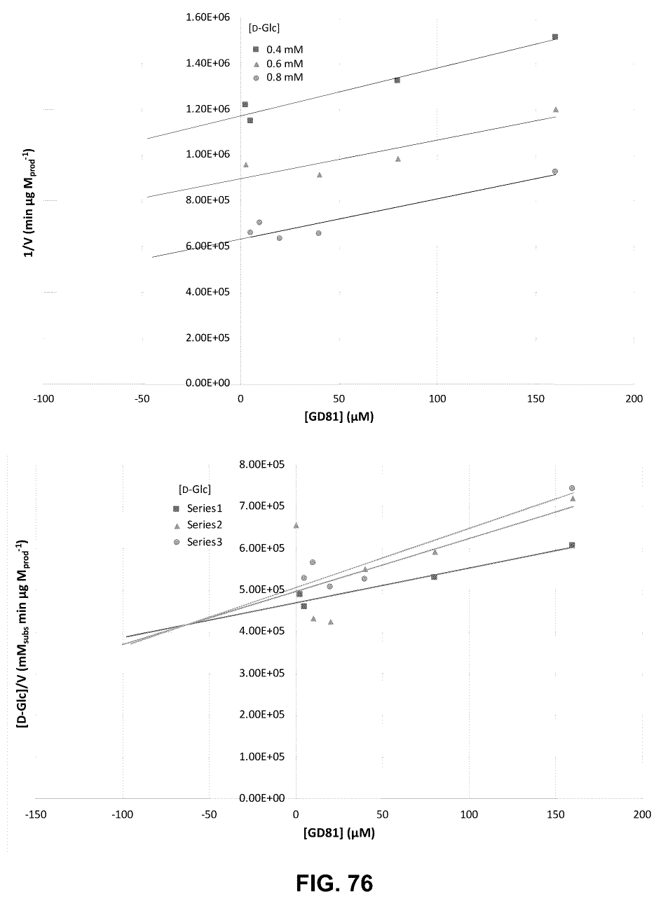

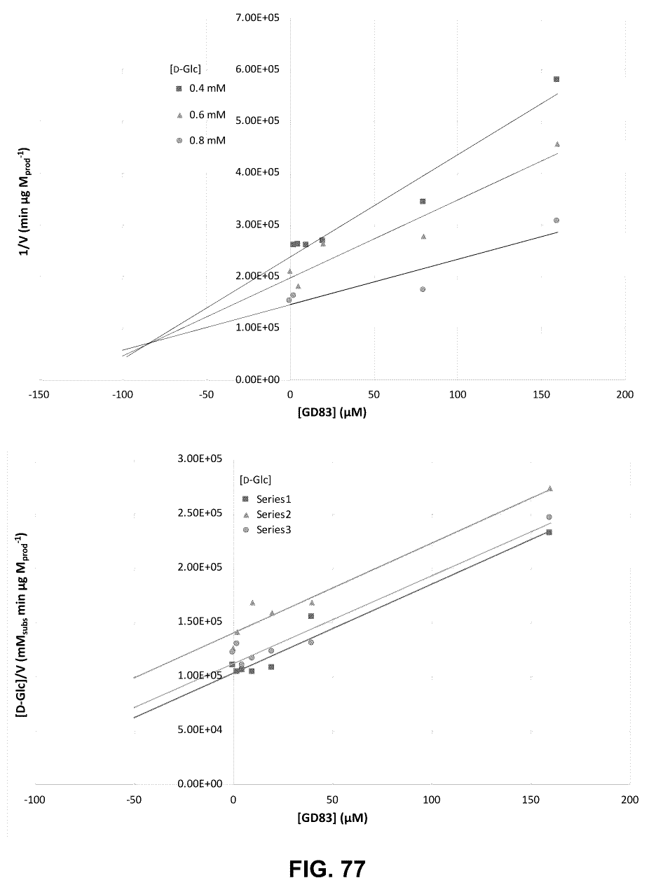

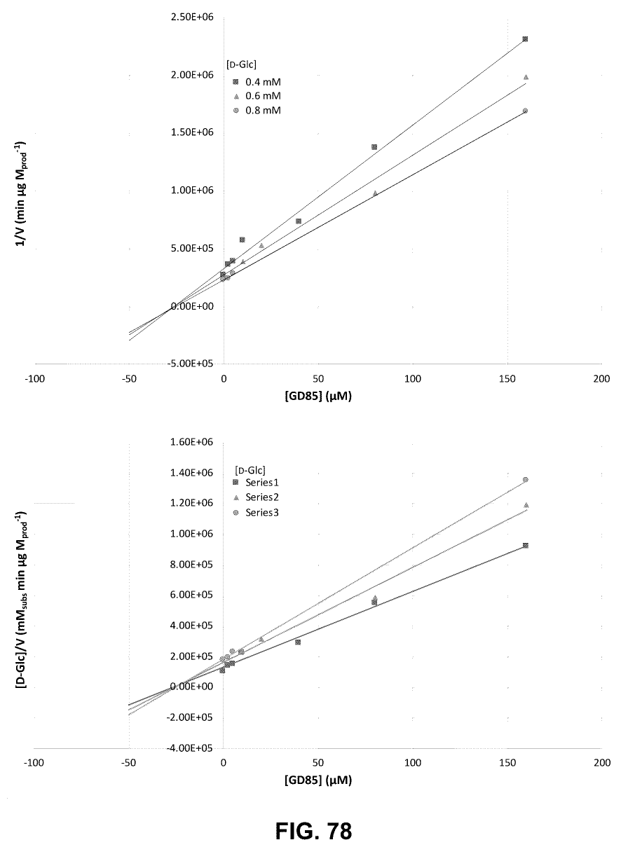

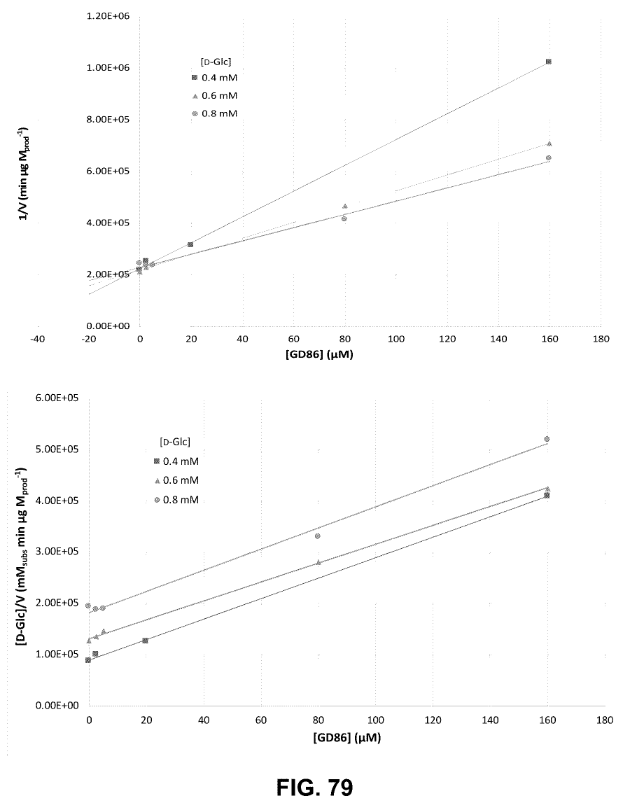

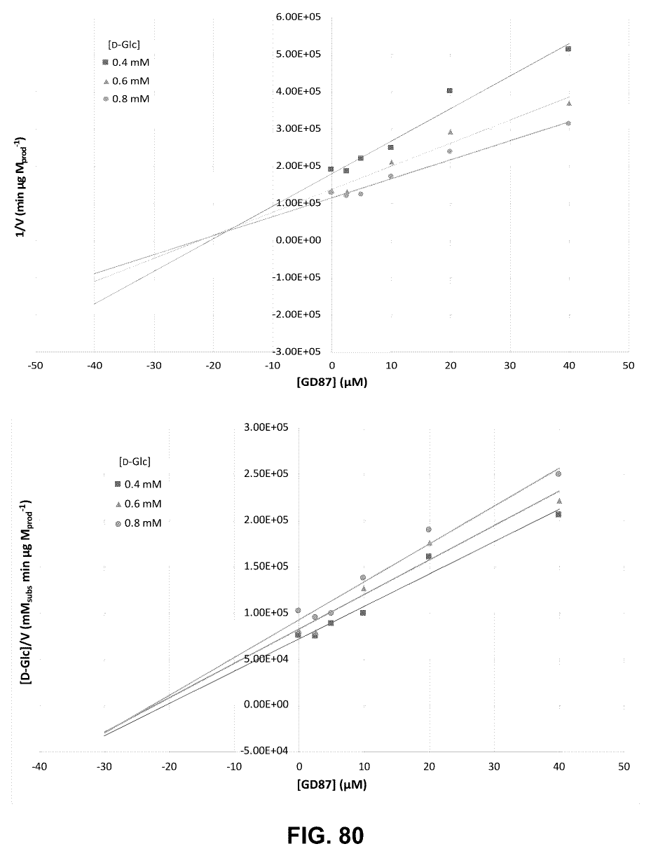

Dixon plots obtained for the examined inhibitors are provided in FIG. 5-FIG. 26. Inhibition constants for each tested compound is shown below in Table 1.

TABLE 1

| Inhibitor | FIG. | | |
|---|---|---|---|
| number | number | Structure | Kᵢ (μM) |
| 1 | 5 | 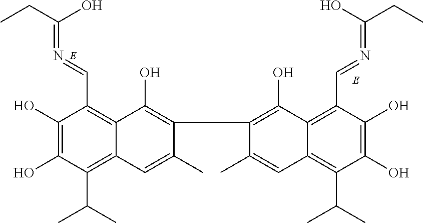 | 80.6 |
| comparative | 6 | 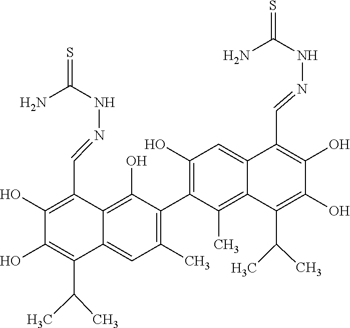 [SMILES: Cc1cc2c(C(C)C)c(O)c(O)c(/C=N/NC(N)=S)c2c(O)c1-c1c(O)cc2c(/C=N/NC(N)=S)c(O)c(O)c(C(C)C)c2c1C] | — |
| 3 | 7 | 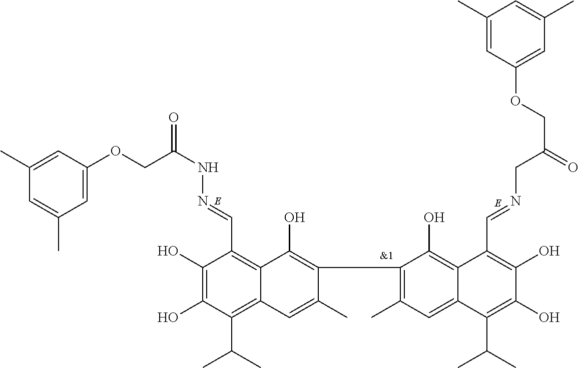 | 60.4 |
| 4 | 8 | 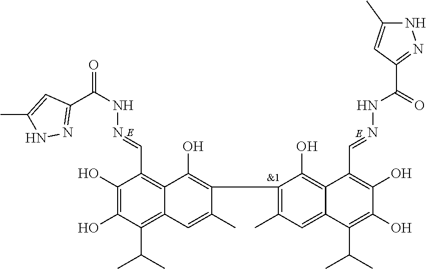 [SMILES: Cc1cc(C(=O)N/N=C/c2c(O)c(O)c(C(C)C)c3cc(C)c(-c4c(C)cc5c(C(C)C)c(O)c(O)c(/C=N/NC(=O)c6cc(C)[nH]n6)c5c4O)c(O)c23)n[nH]1] | 19.12 |
| 5 | 9 | 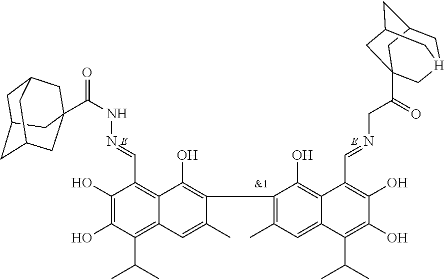 | 775.32 |
| 6 | 10 | 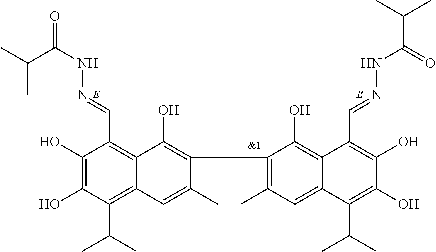 [SMILES: Cc1cc2c(C(C)C)c(O)c(O)c(/C=N/NC(=O)C(C)C)c2c(O)c1-c1c(C)cc2c(C(C)C)c(O)c(O)c(/C=N/NC(=O)C(C)C)c2c1O] | 30.24 |
| 7 | 11 | 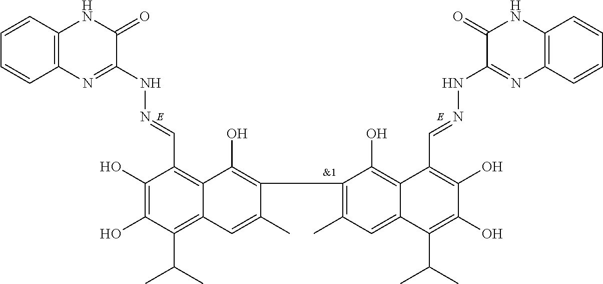 [SMILES: Cc1cc2c(C(C)C)c(O)c(O)c(/C=N/Nc3nc4ccccc4[nH]c3=O)c2c(O)c1-c1c(C)cc2c(C(C)C)c(O)c(O)c(/C=N/Nc3nc4ccccc4[nH]c3=O)c2c1O] | 3.83 |
| 8 | 12 | 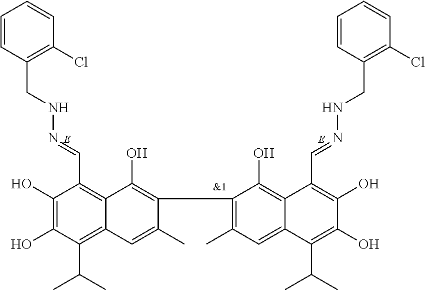 [SMILES: Cc1cc2c(C(C)C)c(O)c(O)c(/C=N/NCc3ccccc3Cl)c2c(O)c1-c1c(C)cc2c(C(C)C)c(O)c(O)c(/C=N/NCc3ccccc3Cl)c2c1O] | 56.3 |
| 9 | 13 | 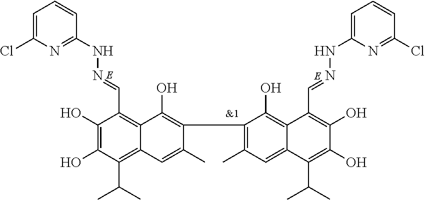 [SMILES: Cc1cc2c(C(C)C)c(O)c(O)c(/C=N/Nc3cccc(Cl)n3)c2c(O)c1-c1c(C)cc2c(C(C)C)c(O)c(O)c(/C=N/Nc3cccc(Cl)n3)c2c1O] | 11 |
| 10 | 14 | 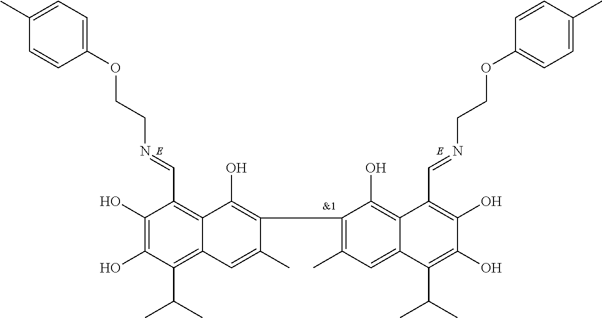 | 3.4 |
| 11 | 15 | 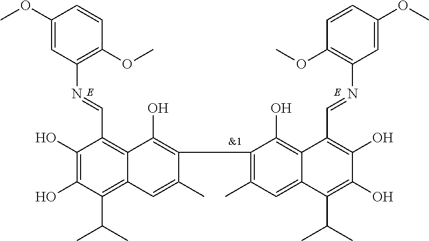 | 30.47 |
| 12 | 16 | 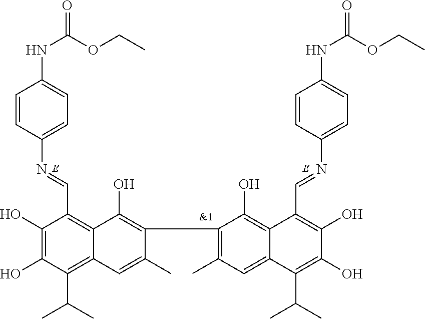 [SMILES: CCOC(=O)Nc1ccc(/N=C/c2c(O)c(O)c(C(C)C)c3cc(C)c(-c4c(C)cc5c(C(C)C)c(O)c(O)c(/C=N/c6ccc(NC(=O)OCC)cc6)c5c4O)c(O)c23)cc1] | 4706.8 |
| 13 | 17 | 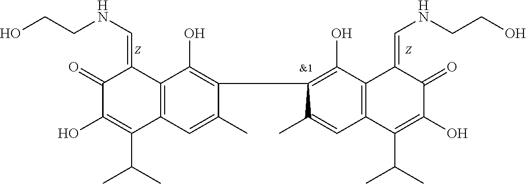 | 422.24 |
| 14 | 18 | 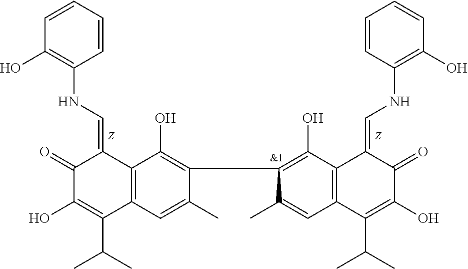 | 467.82 |
| 15 | 27 | 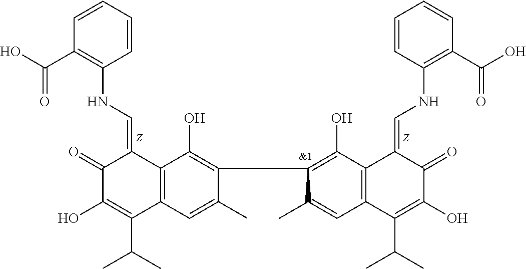 | 1.2 |
| 16 | 28 | 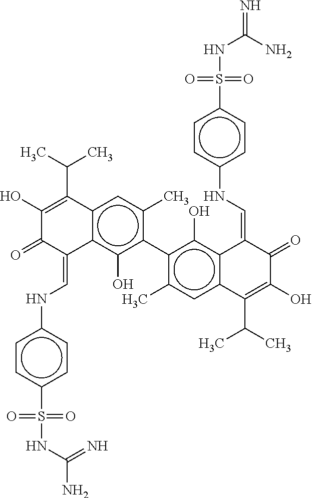 | 1.03 |
| 17 | 19 | 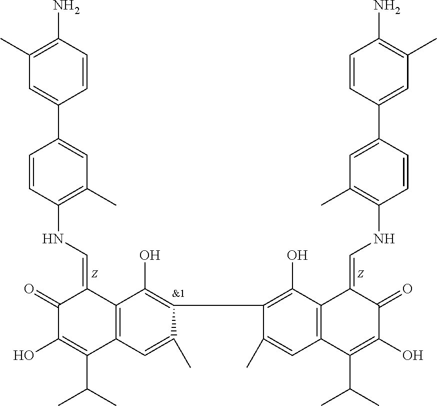 | 531.77 |
| 18 | 20 | 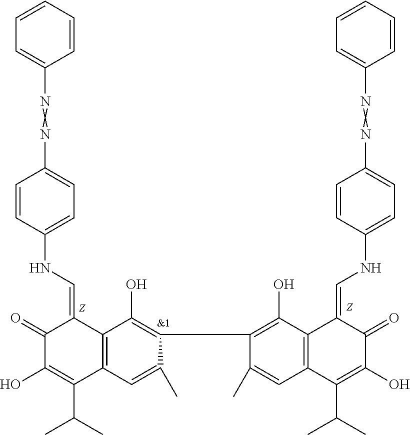 | 45.5 |
| 19 | 21 | 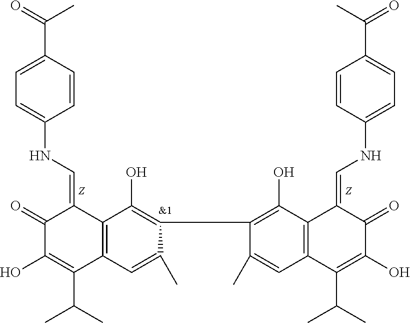 | 10.4 |
| 20 | 22 | 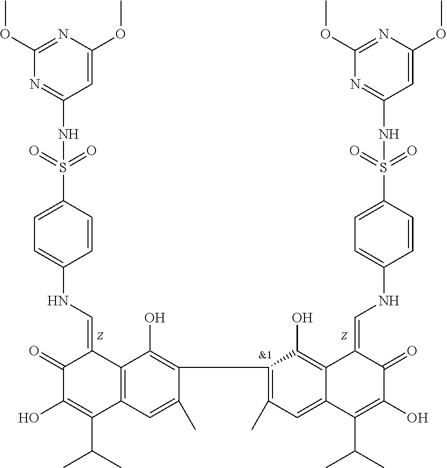 | 11.2 |
| 21 | 23 | 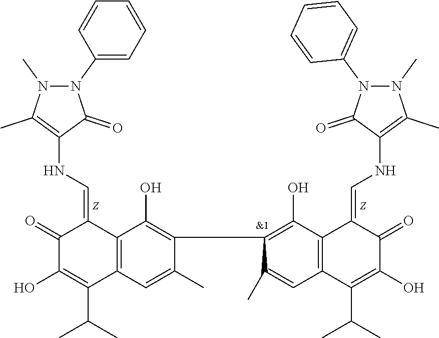 | 9.5 |
| 22 | 24 | 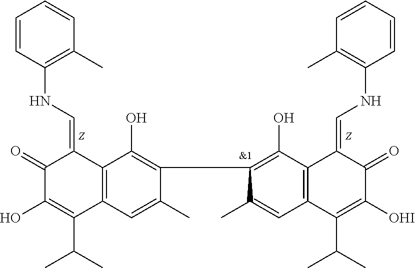 | 24.6 |
| 23 | 25 | 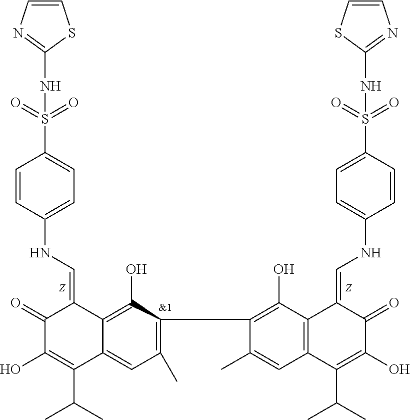 | 32.3 |
| 25 | 26 | 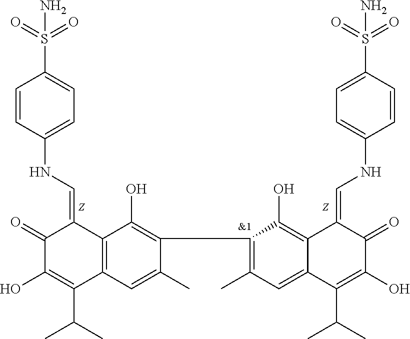 | 74.3 |
| 26 | 29 | 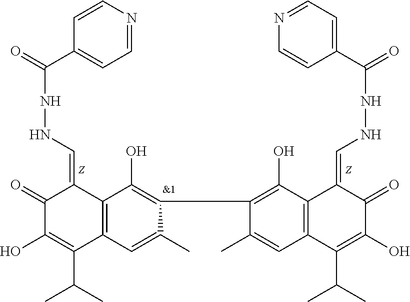 | 212.73 |
| 27 | 30 | 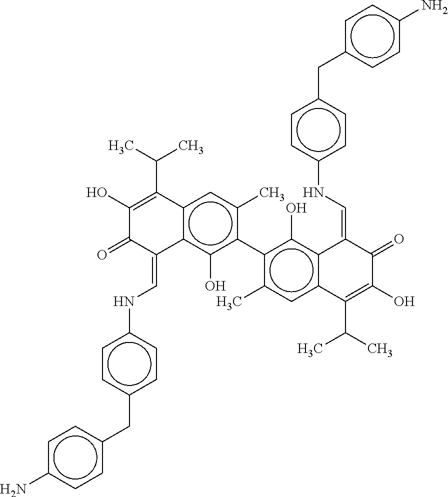 | — |
| 28 | 31 | 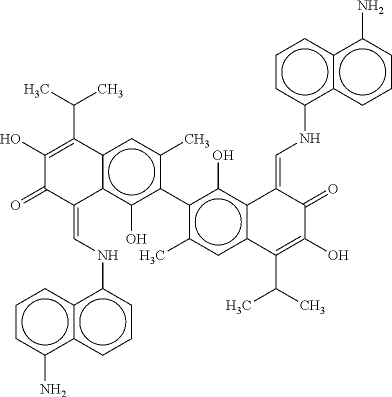 | — |
| 29 | 32 | 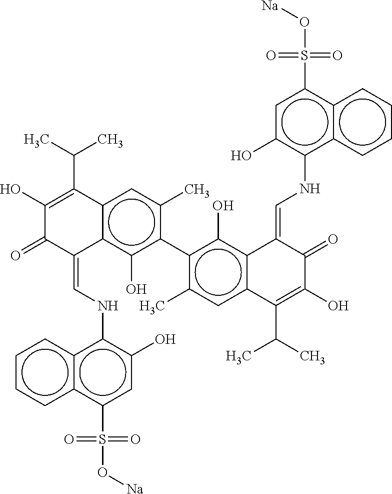 | 9.6 |
| 30 | 33 | 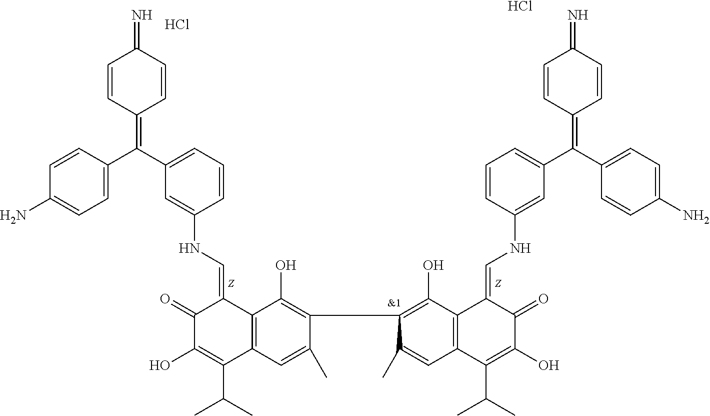 | 3.9 |
| 31 | 34 | 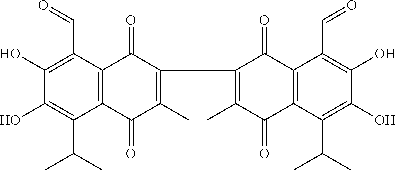 [SMILES: CC1=C(C2=C(C)C(=O)c3c(c(C=O)c(O)c(O)c3C(C)C)C2=O)C(=O)c2c(C=O)c(O)c(O)c(C(C)C)c2C1=O] | 959.63 |
| 32 | — | 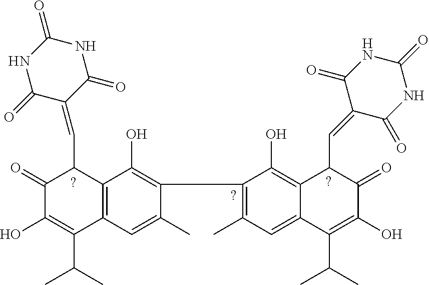 [SMILES: Cc1cc2c(c(O)c1-c1c(C)cc3c(c1O)C(C=C1C(=O)NC(=O)NC1=O)C(=O)C(O)=C3C(C)C)C(C=C1C(=O)NC(=O)NC1=O)C(=O)C(O)=C2C(C)C] | 10.55 |
| 33 | 35 | 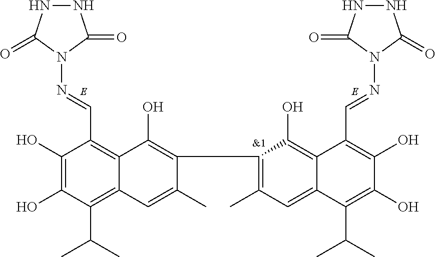 | 121.82 |
| 34 | — | 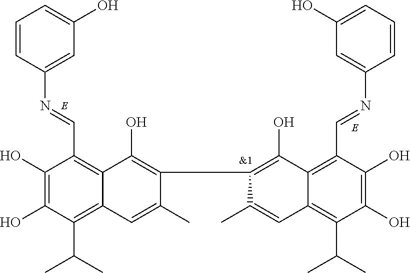 | — |
| 35 | 36 | 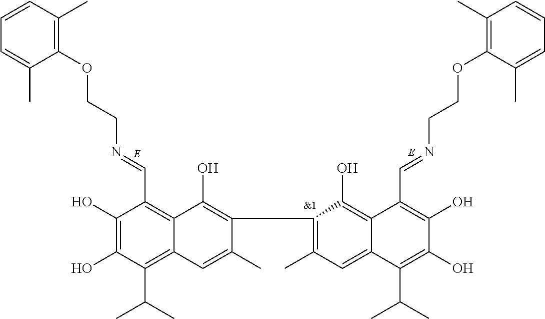 | — |
| 36 | 37 | 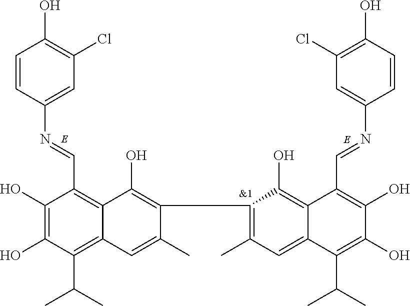 | — |
| 37 | 38 | 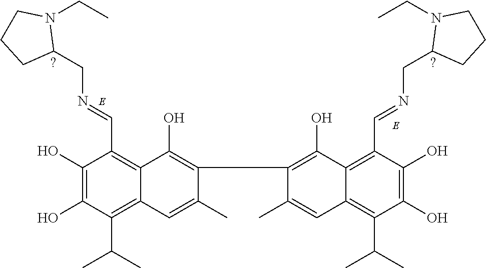 [SMILES: CCN1CCCC1C/N=C/c1c(O)c(O)c(C(C)C)c2cc(C)c(-c3c(C)cc4c(C(C)C)c(O)c(O)c(/C=N/CC5CCCN5CC)c4c3O)c(O)c12] | — |
| 38 | 39 | 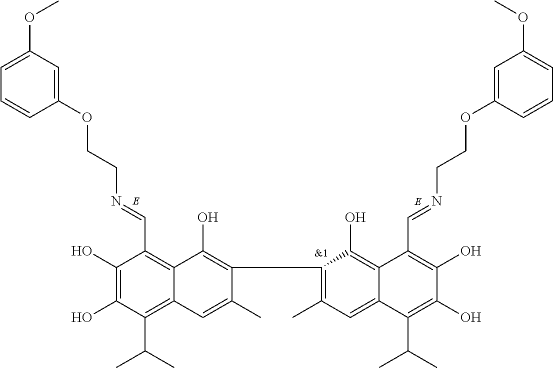 | — |
| 39 | 40 | 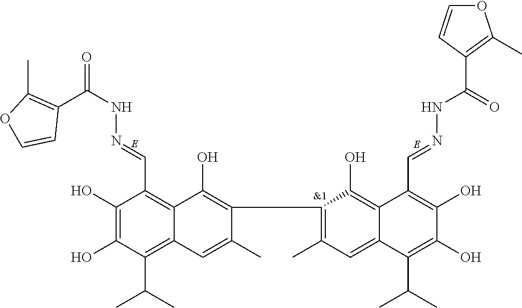 | 9.16 |
| 40 | 41 | 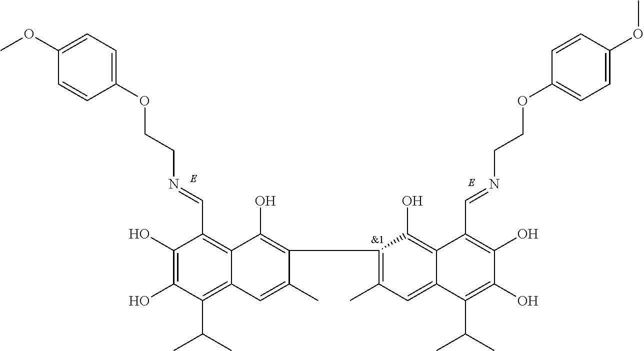 | 29.4 |
| 41 | 42 | 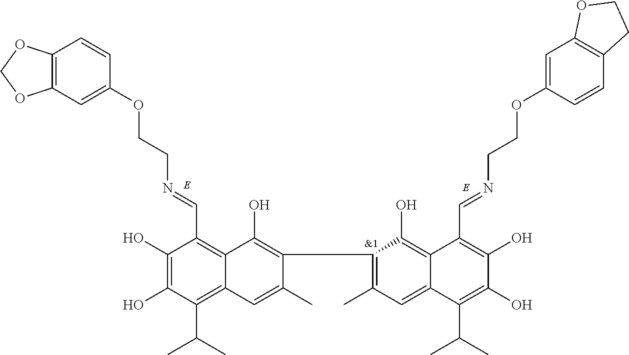 | 327.56 |
| 42 | | 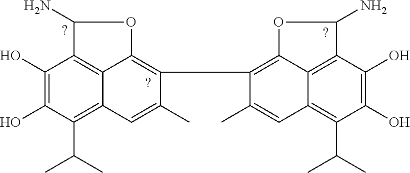 [SMILES: Cc1cc2c(C(C)C)c(O)c(O)c3c2c(c1-c1c(C)cc2c(C(C)C)c(O)c(O)c4c2c1OC4N)OC3N] | — |
| 43 | 43 | 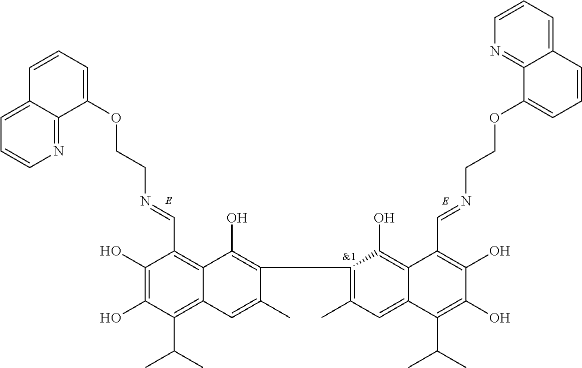 | — |
| 44 | — | 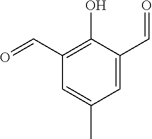 | 99.1 |
| 45 | 45 | 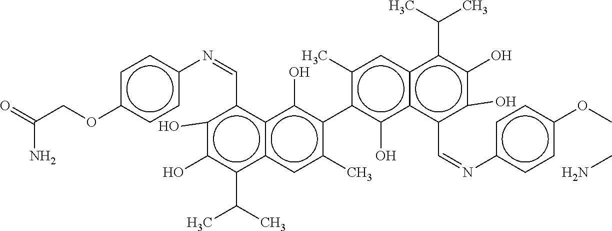 | 13.32 |
| 46 | 46 | 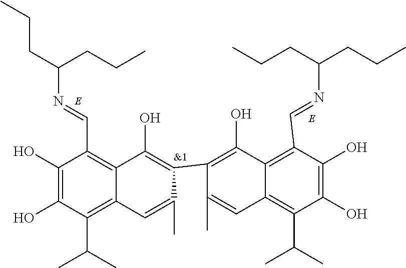 | 99.1 |
| 47 | 47 | 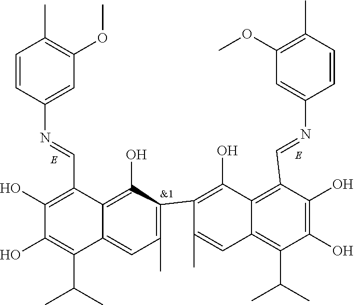 | 29.06 |
| 48 | — | 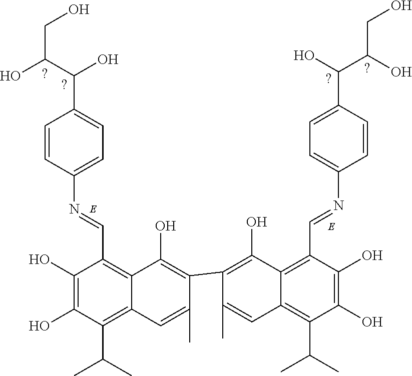 [SMILES: Cc1cc2c(C(C)C)c(O)c(O)c(/C=N/c3ccc(C(O)C(O)CO)cc3)c2c(O)c1-c1c(C)cc2c(C(C)C)c(O)c(O)c(/C=N/c3ccc(C(O)C(O)CO)cc3)c2c1O] | 274.29 |
| 49 | 48 | 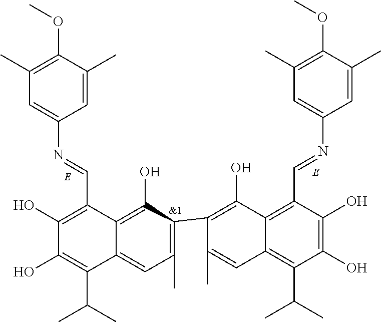 | 16.12 |
| 50 | 49 | 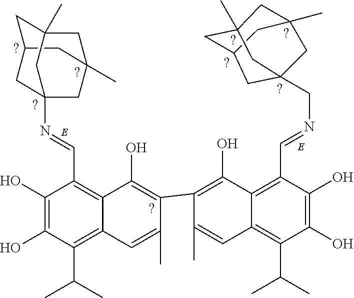 [SMILES: Cc1cc2c(C(C)C)c(O)c(O)c(/C=N/CC34CC5CC(C)(CC(C)(C5)C3)C4)c2c(O)c1-c1c(C)cc2c(C(C)C)c(O)c(O)c(/C=N/C34CC5CC(C)(CC(C)(C5)C3)C4)c2c1O] | 51.27 |
| 51 | 50 | 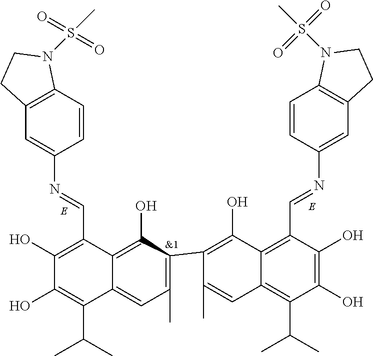 | 0.80 |
| 52 | — | 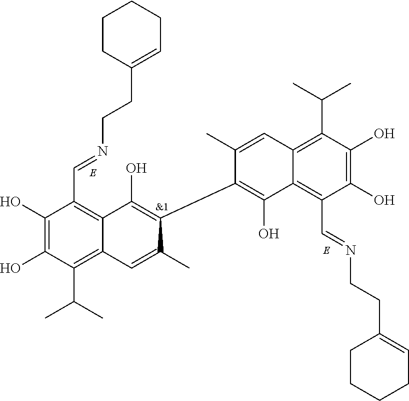 | — |
| 53 | 51 | 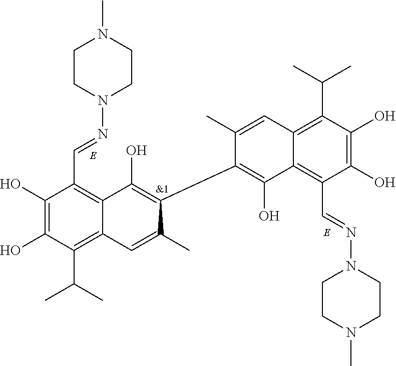 | — |
| 54 | 52 | 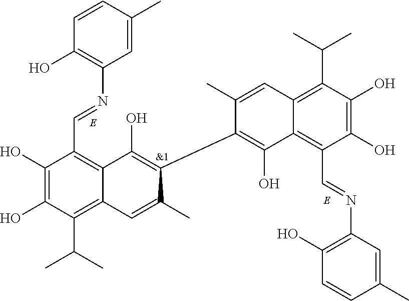 | 3.02 |
| 55 | 53 | 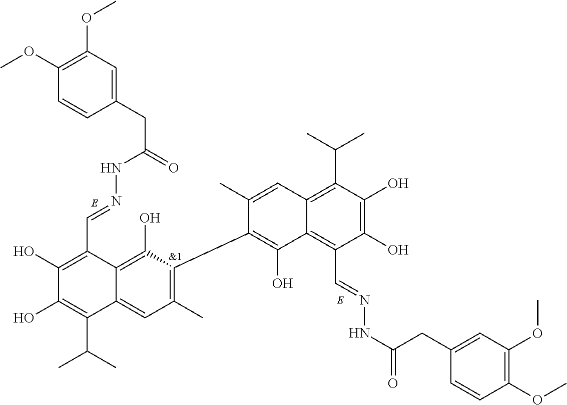 | 41.64 |
| 56 | — | 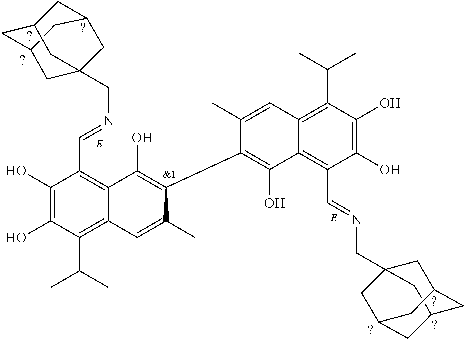 | — |
| 57 | 54 | 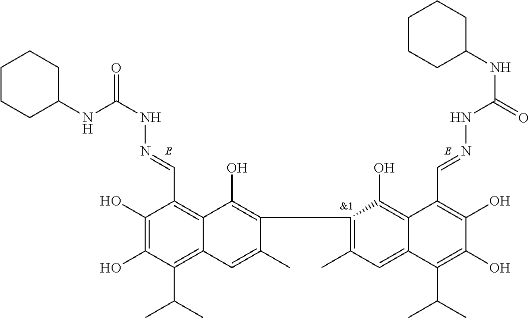 | — |
| 58 | 55 | 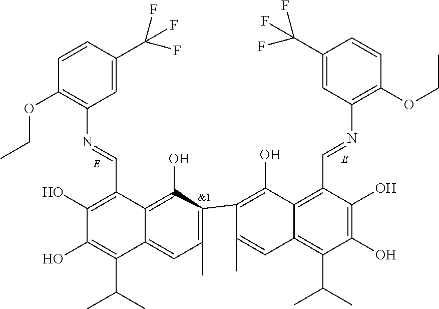 | — |
| 59 | 56 | 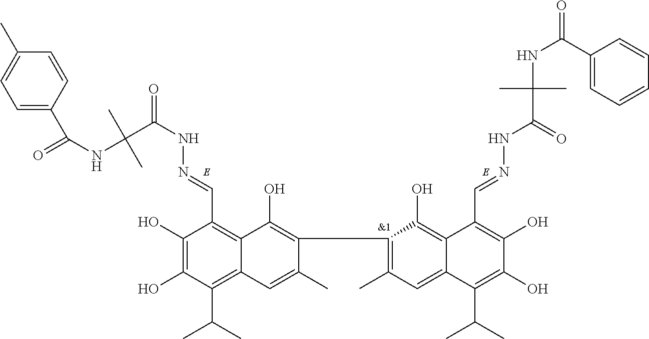 | — |
| 60 | 57 | 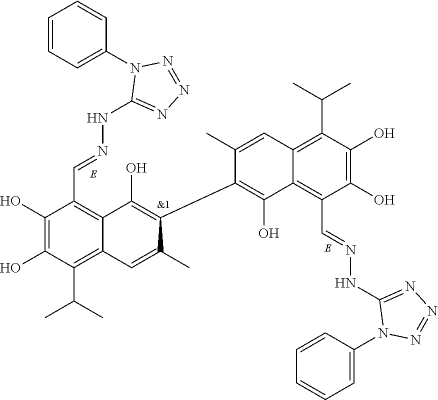 | 53.3 |
| 61 | 58 | 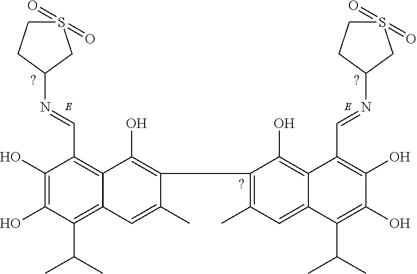 [SMILES: Cc1cc2c(C(C)C)c(O)c(O)c(/C=N/C3CCS(=O)(=O)C3)c2c(O)c1-c1c(C)cc2c(C(C)C)c(O)c(O)c(/C=N/C3CCS(=O)(=O)C3)c2c1O] | — |
| 62 | — | 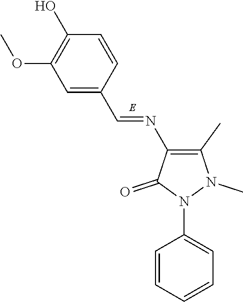 | 19.88 |
| 63 | 59 | 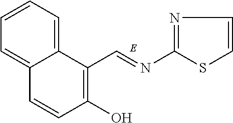 [SMILES: Oc1ccc2ccccc2c1/C=N/c1nccs1] | — |
| 64 | 60 | 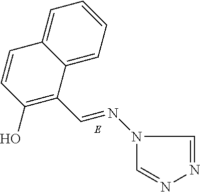 [SMILES: Oc1ccc2ccccc2c1/C=N/n1cnnc1] | — |
| 65 | 61 | 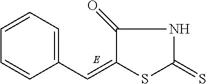 [SMILES: O=C1NC(=S)S/C1=C/c1ccccc1] | 43.29 |
| 66 | 62 | 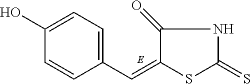 [SMILES: O=C1NC(=S)S/C1=C/c1ccc(O)cc1] | 14.69 |
| 67 | 63 | 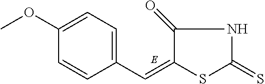 [SMILES: COc1ccc(/C=C2/SC(=S)NC2=O)cc1] | — |
| 68 | 64 | 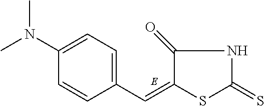 [SMILES: CN(C)c1ccc(/C=C2/SC(=S)NC2=O)cc1] | 0.27 |
| 69 | 65 | 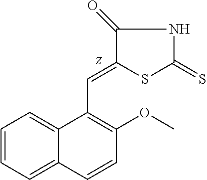 [SMILES: COc1ccc2ccccc2c1/C=C1\SC(=S)NC1=O] | 12.86 |
| 70 | 66 | 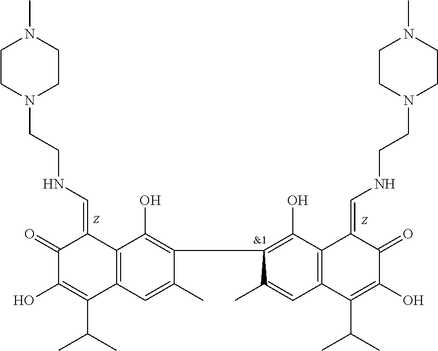 | — |
| 71 | 67 | 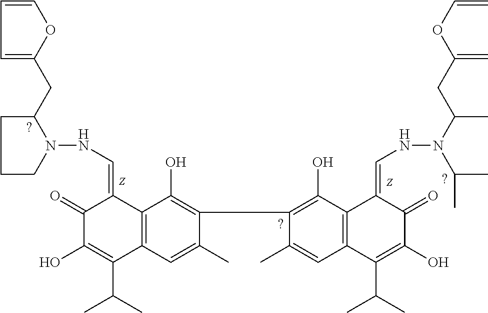 [SMILES: C=COC(=C)CC(C)N(N/C=C1\C(=O)C(O)=C(C(C)C)c2cc(C)c(-c3c(C)cc4c(c3O)/C(=C/NN3CCCC3Cc3ccco3)C(=O)C(O)=C4C(C)C)c(O)c21)C(C)C] | — |
| 72 | 68 | 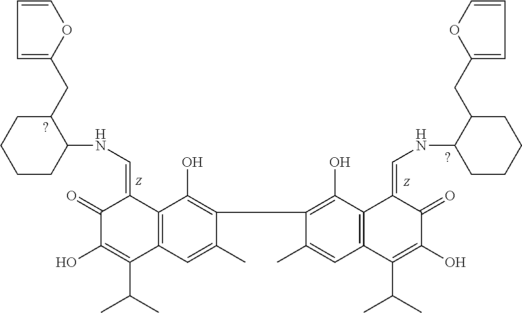 [SMILES: Cc1cc2c(c(O)c1-c1c(C)cc3c(c1O)/C(=C/NC1CCCCC1Cc1ccco1)C(=O)C(O)=C3C(C)C)/C(=C/NC1CCCCC1Cc1ccco1)C(=O)C(O)=C2C(C)C] | — |
| 73 | 69 | 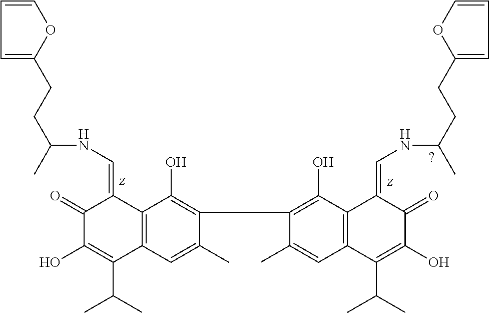 [SMILES: Cc1cc2c(c(O)c1-c1c(C)cc3c(c1O)/C(=C/NC(C)CCc1ccco1)C(=O)C(O)=C3C(C)C)/C(=C/NC(C)CCc1ccco1)C(=O)C(O)=C2C(C)C] | 2.03 |
| 74 | 70 | 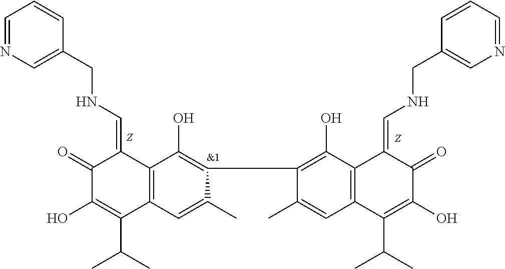 | 123.44 |
| 75 | 71 | 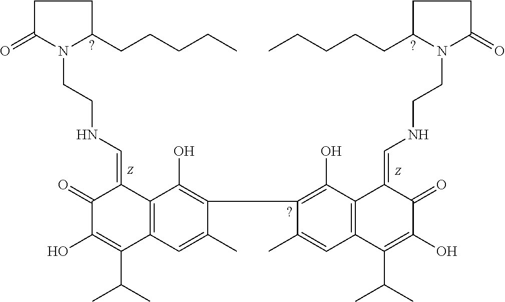 [SMILES: CCCCCC1CCC(=O)N1CCN/C=C1\C(=O)C(O)=C(C(C)C)c2cc(C)c(-c3c(C)cc4c(c3O)/C(=C/NCCN3C(=O)CCC3CCCCC)C(=O)C(O)=C4C(C)C)c(O)c21] | 560.36 |
| 76 | 72 | 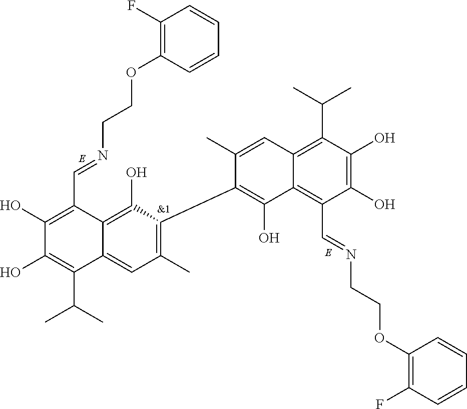 | 104.5 |
| 77 | 73 | 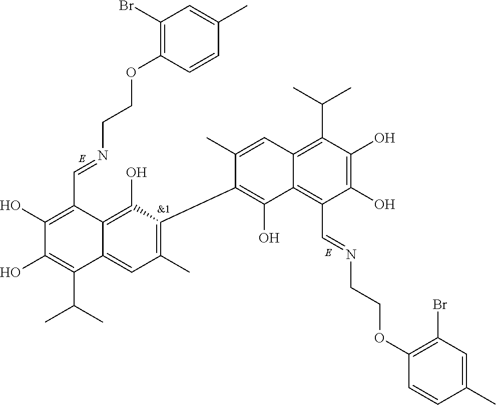 | 42.25 |
| 78 | 74 | 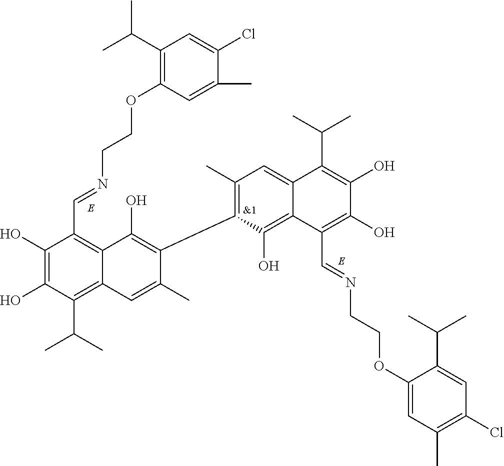 | — |
| 79 | 75 | 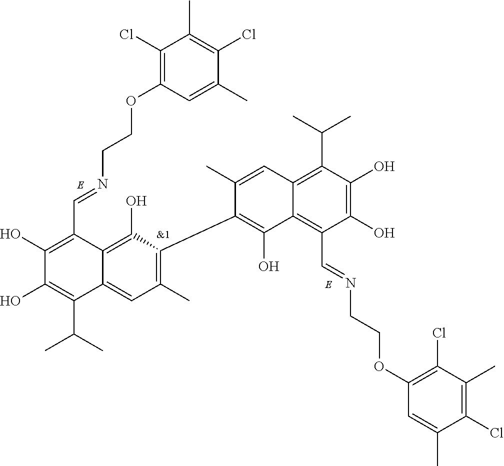 | 126.87 |
| 80 | — | 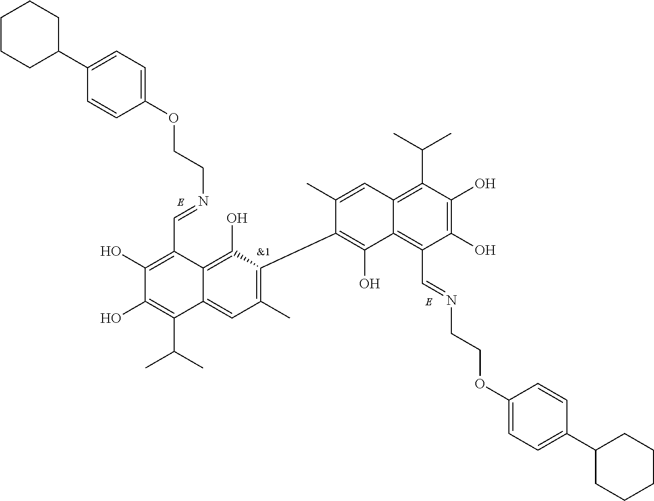 | 11.78 |
| 81 | 76 | 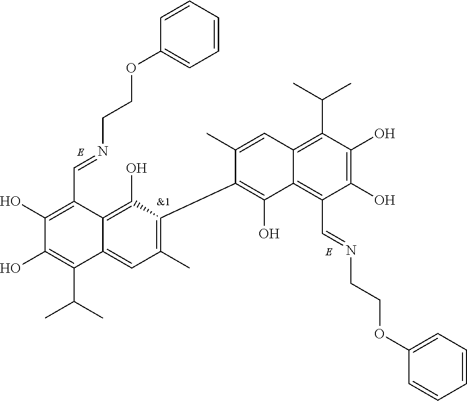 | — |
| 82 | — | 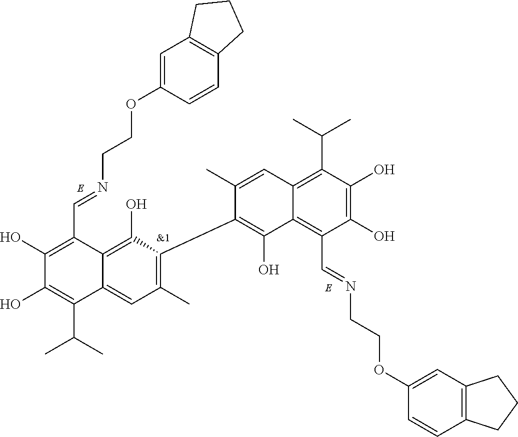 | 98.88 |
| 83 | 77 | 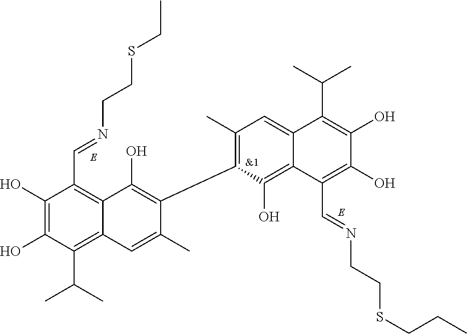 | 84.54 |
| 84 | — | 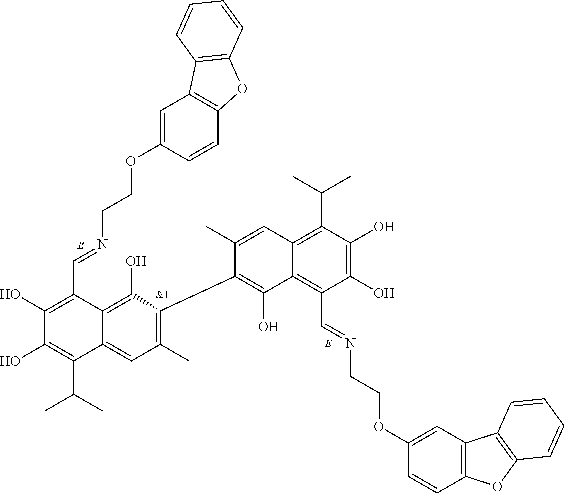 | — |
| 85 | 78 | 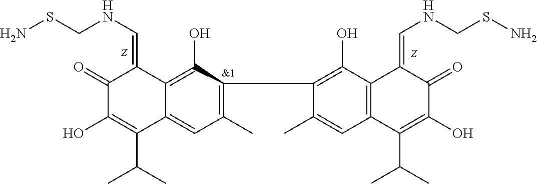 | 30.13 |
| 86 | 79 | 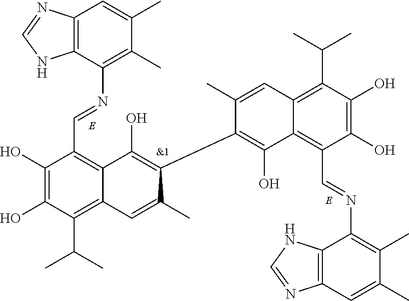 | 2.58 |
| 87 | 80 | 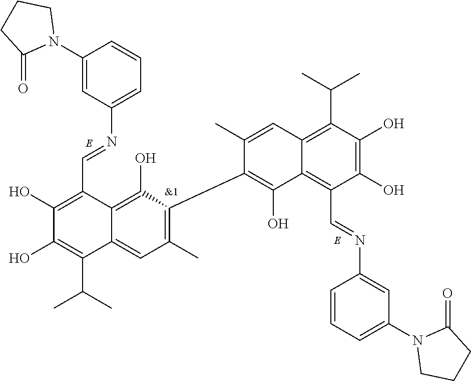 | 17.58 |
| 88 | — | 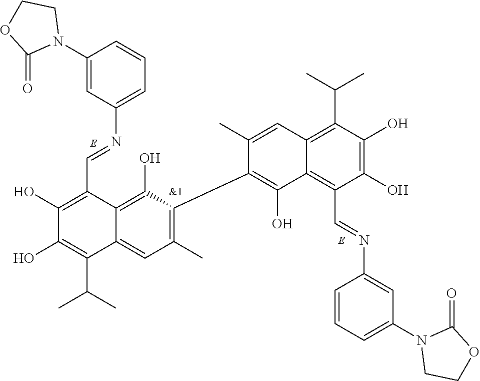 | — |

These and other modifications and variations to the present invention may be practiced by those of ordinary skill in the art, without departing from the spirit and scope of the present invention, which is more particularly set forth in the appended claims. In addition, it should be understood the aspects of the various embodiments may be interchanged both in whole or in part. Furthermore, those of ordinary skill in the art will appreciate that the foregoing description is by way of example only, and is not intended to limit the invention so further described in the appended claims.

What is claimed:

1. A method of inhibiting a parasitic hexokinase or glucokinase, the method comprising delivering a compound to a parasite, the compound comprising one or more of the following:
a) a gossypol derivative having the following structure: 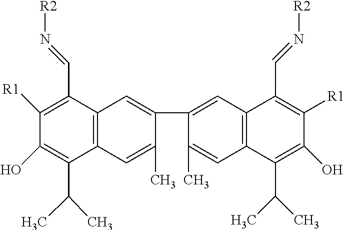
in which R1 is oxygen or hydroxyl and R2 includes a substituted phenyl group, a substituted conjugated or unconjugated cyclopentyl or cyclooctyl group, a substituted cyclohexyl group, or a substituted or unsubstituted C3-C7 group;
b) a gossypol derivative having the following structure: 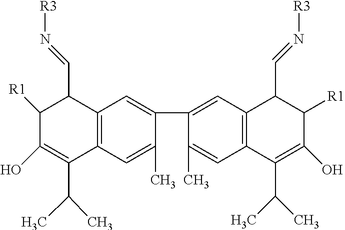
in which R1 is oxygen or hydroxyl and R3 is selected from one of the following: 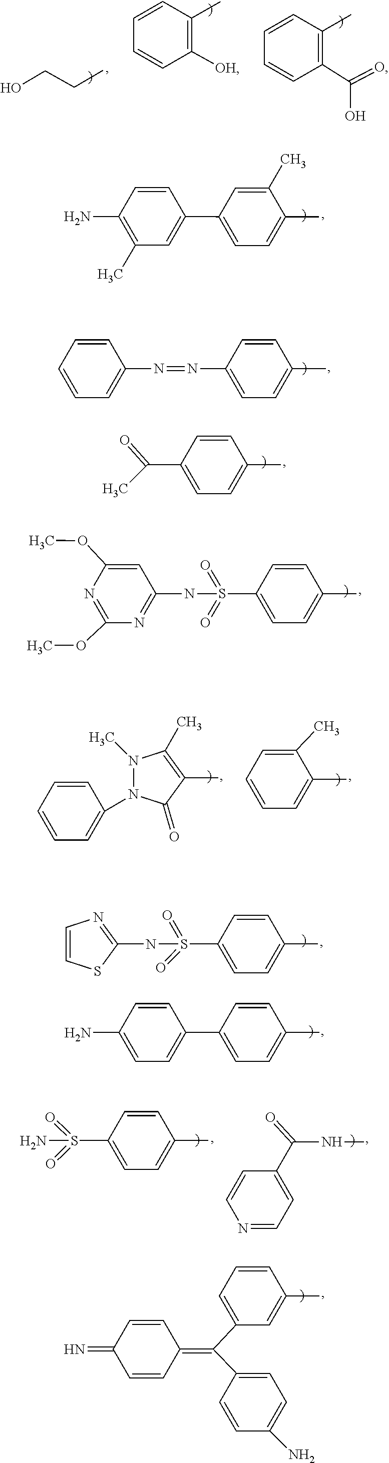 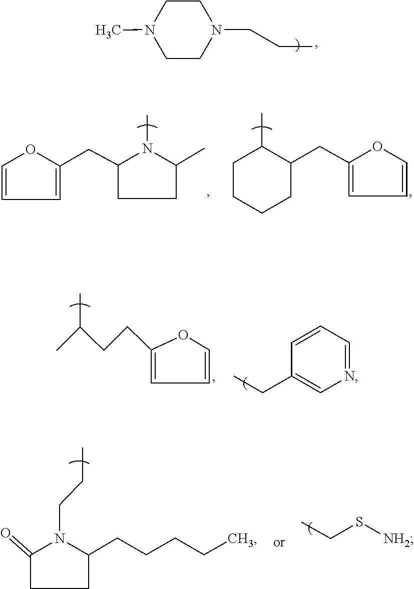
c) a gossypol derivative having the following structure: 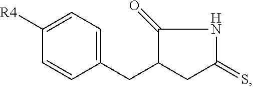
in which R4 is selected from hydrogen, hydroxyl, methoxy, or amine and optionally, the phenyl group is instead a naphthyl group; or
d) a gossypol derivative having the following structure:

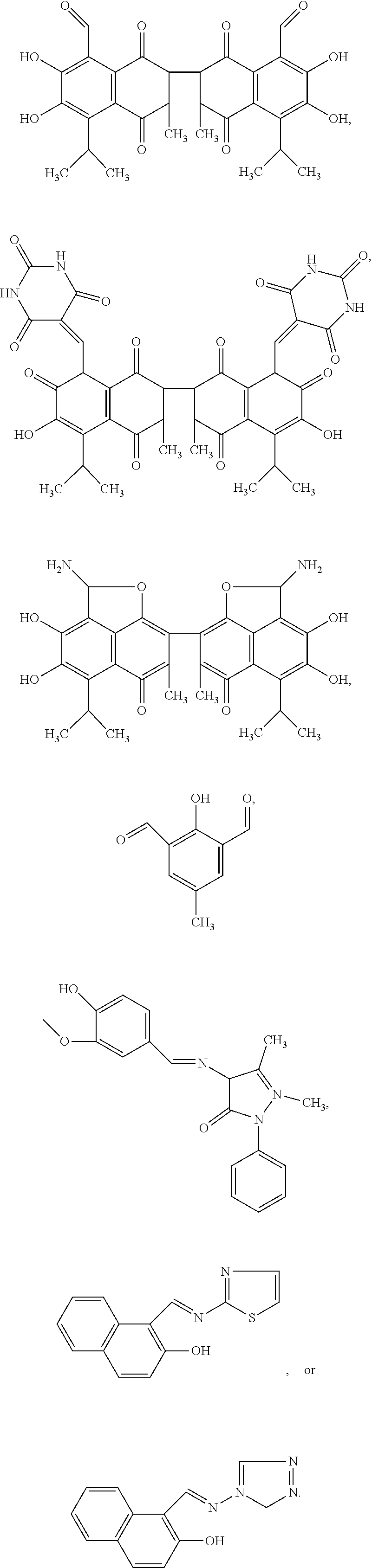

2. The method of claim 1, wherein the parasite comprises a kinetoplastid parasite.

3. The method of claim 1, wherein the parasite comprises Trypanosoma cruzi, Trypanosoma brucei, or Leishmania spp.

4. The method of claim 1, wherein the parasite comprises Trypanosoma cruzi.

5. The method of claim 1, wherein the parasitic hexokinase or glucokinase comprises Trypanosoma cruzi glucokinase.

6. The method of claim 1, wherein the compound comprises:
a gossypol derivative having the following structure: 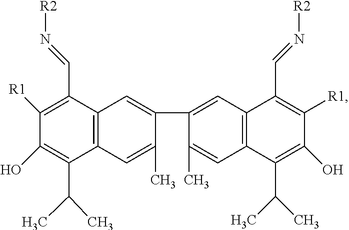
in which R1 is oxygen or hydroxyl and R2 includes a substituted phenyl group, a substituted conjugated or unconjugated cyclopentyl or cyclooctyl group, a substituted cyclohexyl group, or a substituted or unsubstituted C3-C7 group.

7. The method of claim 1, wherein the compound comprises:
a gossypol derivative having the following structure: 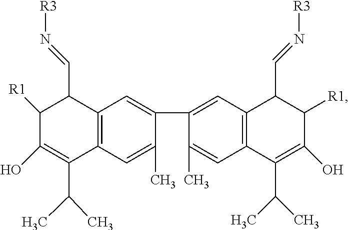
in which R1 is oxygen or hydroxyl and R3 is selected from one of the following:

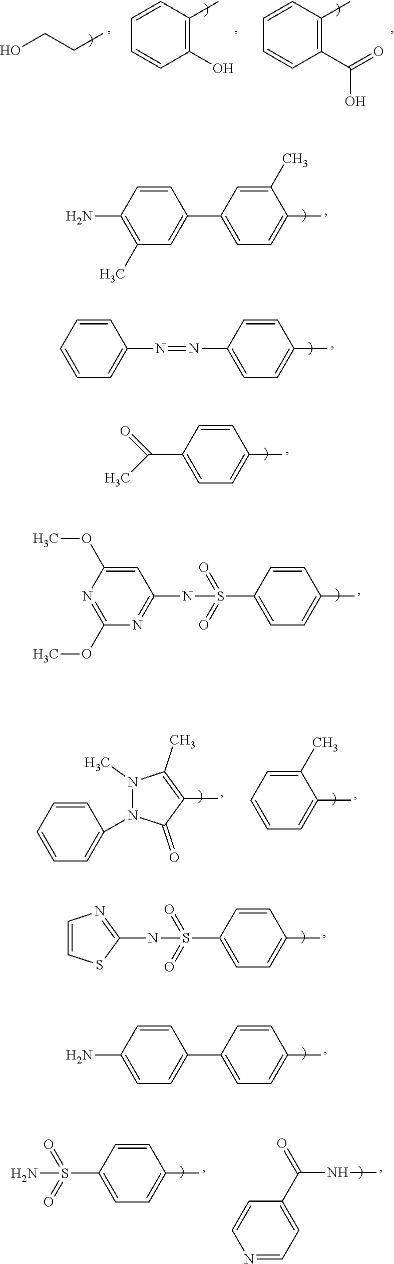

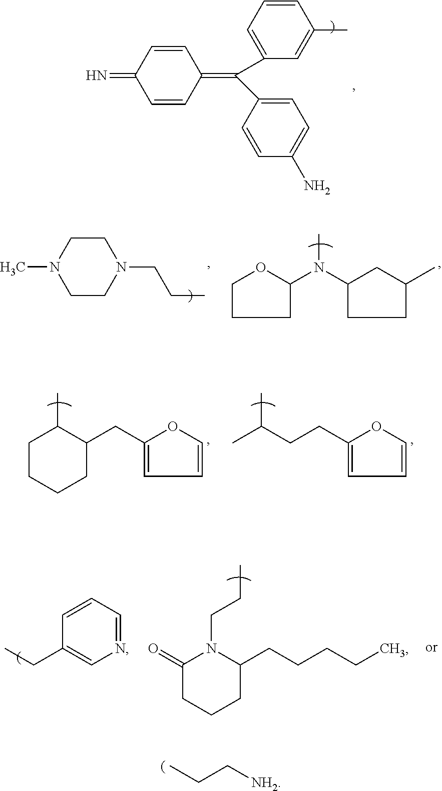

8. The method of claim 1, wherein the compound comprises:
a gossypol derivative having the following structure: 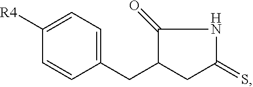
in which R4 is selected from hydrogen, hydroxyl, methoxy, or amine and optionally, the phenyl group is instead a naphthyl group.

9. The method of claim 1, wherein the compound comprises:
a gossypol derivative having the following structure:

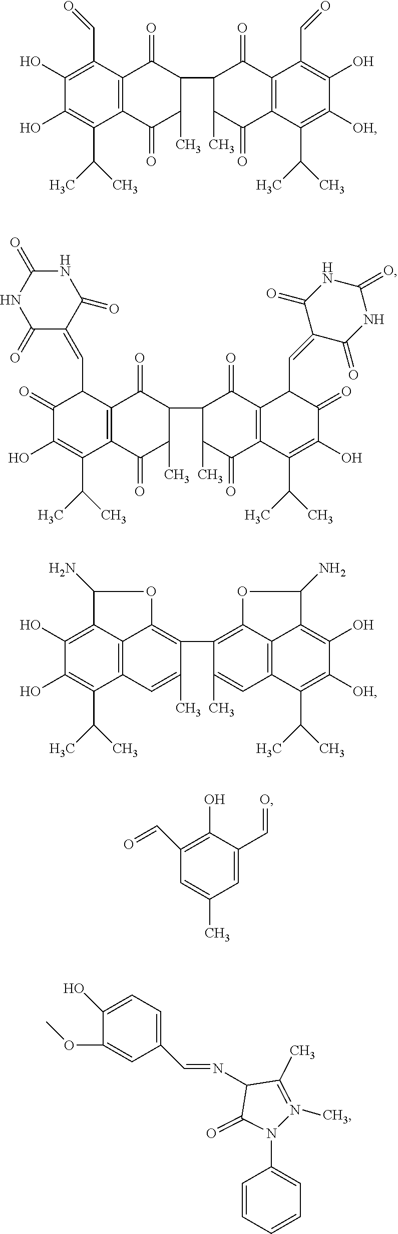

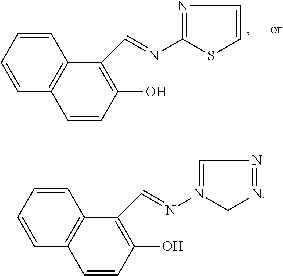

10. A method for treating a subject that is infected by a parasitic organism, the method comprising administering a pharmaceutical composition to the subject, the pharmaceutical composition comprising a pharmaceutically acceptable carrier and a compound comprising one or more of the following:
a) a gossypol derivative having the following structure: 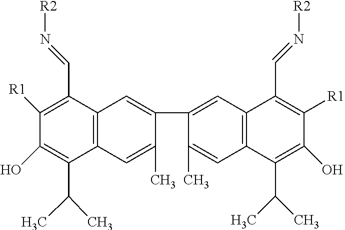
in which R1 is oxygen or hydroxyl and R2 includes a substituted phenyl group, a substituted conjugated or unconjugated cyclopentyl or cyclooctyl group, a substituted cyclohexyl group, or a substituted or unsubstituted C3-C7 group;
b) a gossypol derivative having the following structure: 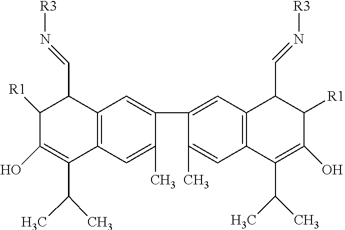
in which R1 is oxygen or hydroxyl and R3 is selected from one of the following: 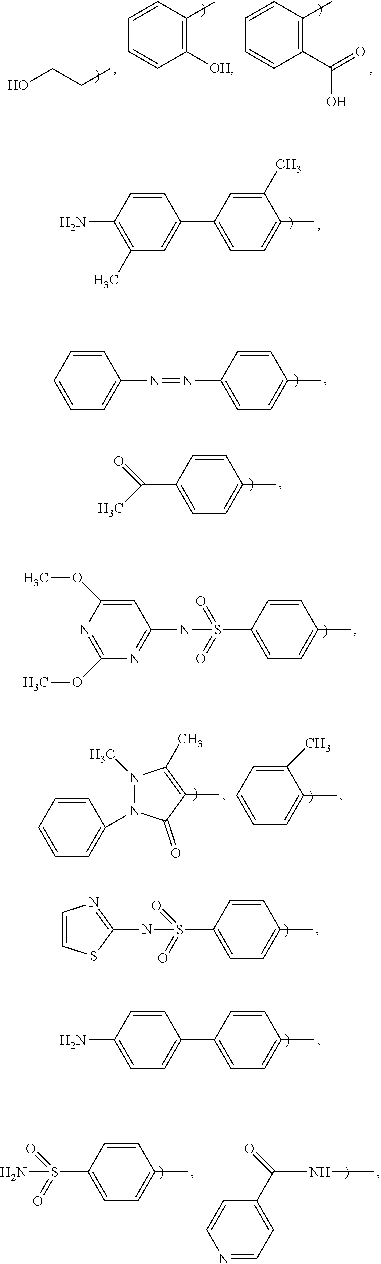 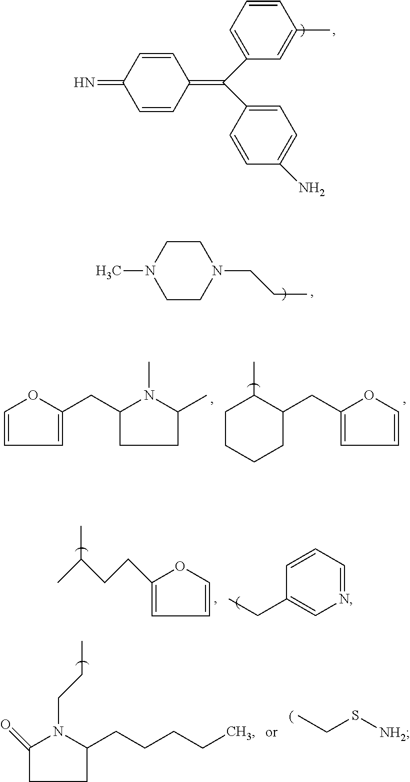
c) a gossypol derivative having the following structure: 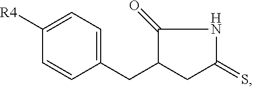
in which R4 is selected from hydrogen, hydroxyl, methoxy, or amine and optionally, the phenyl group is instead a naphthyl group; or
d) a gossypol derivative having the following structure:

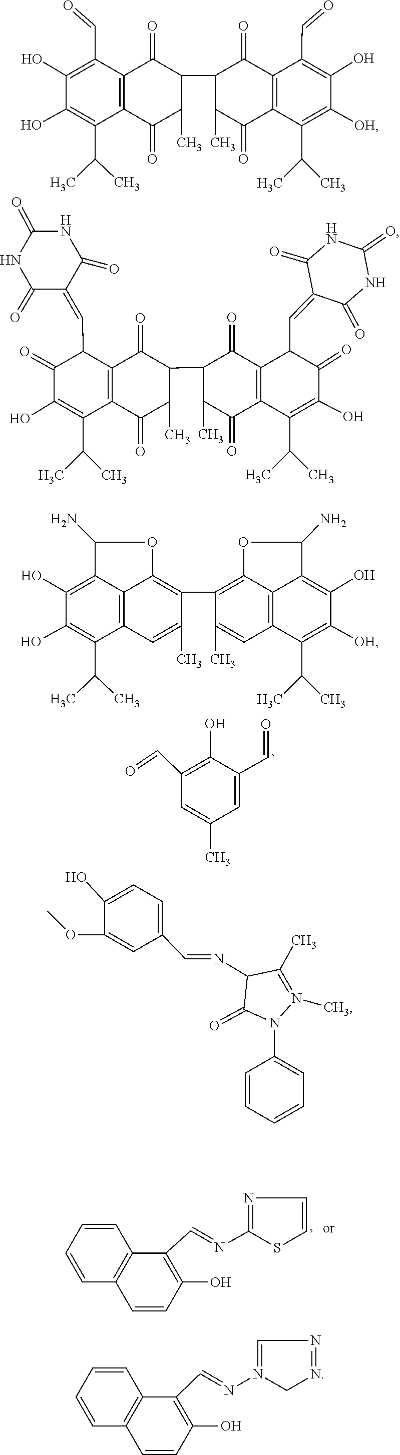

11. The method of claim 10, wherein the pharmaceutical composition is administered via oral administration.

12. The method of claim 10, wherein the pharmaceutical composition is administered via parenteral administration.

13. The method of claim 10, wherein the pharmaceutical composition is administered via topical administration.

14. The method of claim 10, wherein the subject has been diagnosed with a disease associated with the parasitic organism.

15. The method of claim 14, wherein the disease is American Trypanosomiasis, Human African Trypanosomiasis, Leishmaniasis, Malaria, Schistosomaisis, or a Filarial disease.

16. The method of claim 15, wherein the disease is American Trypanosomiasis.

17. The method of claim 15, wherein the disease is Human African Trypanosomiasis.

18. The method of claim 15, wherein the disease is Malaria.

19. The method of claim 10, wherein the subject is a human subject.

20. A pharmaceutical composition comprising a pharmaceutically acceptable carrier and a compound comprising one or more of the following:
a) a gossypol derivative having the following structure: 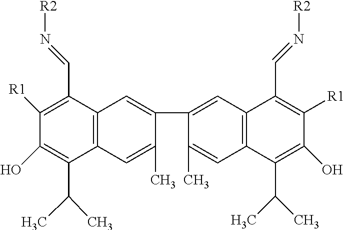
in which R1 is oxygen or hydroxyl and R2 includes a substituted phenyl group, a substituted conjugated or unconjugated cyclopentyl or cyclooctyl group, a substituted cyclohexyl group, or a substituted or unsubstituted C3-C7 group;
b) a gossypol derivative having the following structure: 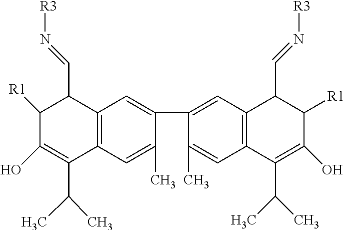
in which R1 is oxygen or hydroxyl and R3 is selected from one of the following: 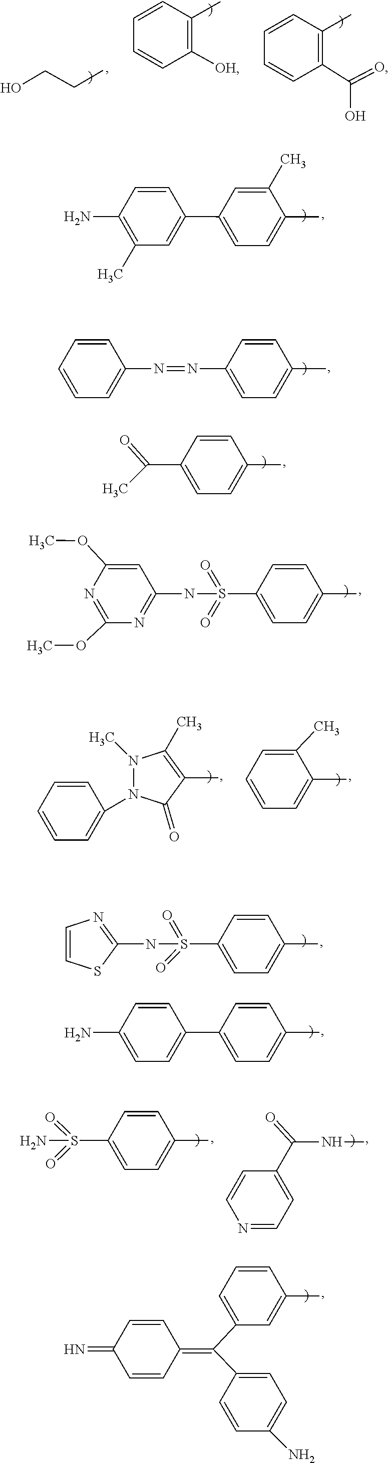 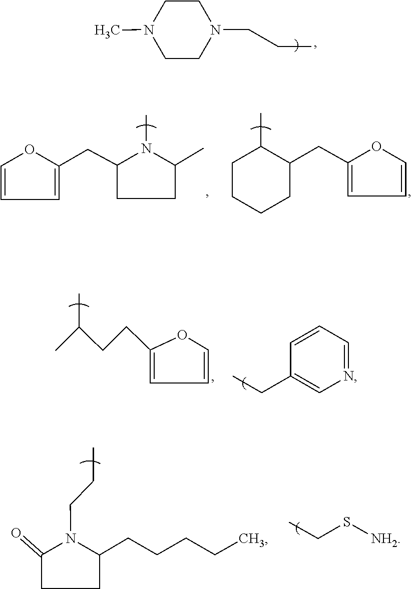
c) a gossypol derivative having the following structure: 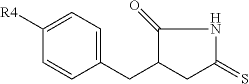
in which R4 is selected from hydrogen, hydroxyl, methoxy, or amine and optionally, the phenyl group is instead a naphthyl group; or
d) a gossypol derivative having the following structure: [chemical structure]
